SEQUENCE LISTING (24 sequences)

SEQ ID NO: 1 - DNA, 21 bp, Artificial Sequence
  Synthetic Polynucleotide
ttcgtacgggcgtttactatc

SEQ ID NO: 2 - DNA, 20 bp, Artificial Sequence
  Synthetic Polynucleotide
tcccgttctacagggaatct

SEQ ID NO: 3 - DNA, 20 bp, Artificial Sequence
  Synthetic Polynucleotide
ccttcattgacctcaactac

SEQ ID NO: 4 - DNA, 20 bp, Artificial Sequence
  Synthetic Polynucleotide
ggaaggccatgccagtgagc

SEQ ID NO: 5 - DNA, 20 bp, Artificial Sequence
  Synthetic Polynucleotide
tcgacagcctctcagtagca

SEQ ID NO: 6 - DNA, 20 bp, Artificial Sequence
  Synthetic Polynucleotide
tgacacacgccttaattcca

SEQ ID NO: 7 - DNA, 20 bp, Artificial Sequence
  Synthetic Polynucleotide
accggtttcatggaagaatg

SEQ ID NO: 8 - DNA, 20 bp, Artificial Sequence
  Synthetic Polynucleotide
ttcactcggcaaacactgag

SEQ ID NO: 9 - DNA, 20 bp, Artificial Sequence
  Synthetic Polynucleotide
ggtccttggctgagtttgaa

SEQ ID NO: 10 - DNA, 21 bp, Artificial Sequence
  Synthetic Polynucleotide
gccatgtagaaccaagtggaa

SEQ ID NO: 11 - DNA, 20 bp, Artificial Sequence
  Synthetic Polynucleotide
ctcggttctccctttcacag

SEQ ID NO: 12 - DNA, 20 bp, Artificial Sequence
  Synthetic Polynucleotide
ccagcaggacgttctttctc

SEQ ID NO: 13 - DNA, 20 bp, Artificial Sequence
  Synthetic Polynucleotide
cgcagagtggatttgaaaca

SEQ ID NO: 14 - DNA, 20 bp, Artificial Sequence
  Synthetic Polynucleotide
cagccagggagaaaagtgac

SEQ ID NO: 15 - DNA, 20 bp, Artificial Sequence
  Synthetic Polynucleotide
tgcttggtcttcatcgtcag

SEQ ID NO: 16 - DNA, 20 bp, Artificial Sequence
  Synthetic Polynucleotide
ataccccacctggcctactc

SEQ ID NO: 17 - DNA, 24 bp, Artificial Sequence
  Synthetic Polynucleotide
tgacaatggtacagagagatcaca

SEQ ID NO: 18 - DNA, 20 bp, Artificial Sequence
  Synthetic Polynucleotide
gctctgggttcttgctgatg

SEQ ID NO: 19 - DNA, 21 bp, Artificial Sequence
  Synthetic Polynucleotide
gggagcaaaatgcagtaagaa

SEQ ID NO: 20 - DNA, 20 bp, Artificial Sequence
  Synthetic Polynucleotide
atcgatgtgcgtagctttca

SEQ ID NO: 21 - DNA, 20 bp, Artificial Sequence
  Synthetic Polynucleotide
cactgcaactgccagagaaa

SEQ ID NO: 22 - DNA, 21 bp, Artificial Sequence
  Synthetic Polynucleotide
tccagactcttggggtattca

SEQ ID NO: 23 - DNA, 20 bp, Artificial Sequence
  Synthetic Polynucleotide
cccgtctatgaaagcaggag

SEQ ID NO: 24 - DNA, 20 bp, Artificial Sequence
  Synthetic Polynucleotide
cacggggattgtttaaatgc

(19) United States
(12) Patent Application Publication
(10) Pub. No.: US 2019/0192532 A1
(43) Pub. Date: Jun. 27, 2019

(54) COMBINATION THERAPY OF BROMODOMAIN INHIBITORS AND CHECKPOINT BLOCKADE

(71) Applicants: DANA-FARBER CANCER INSTITUTE, Boston, MA (US); PETER MACCALLUM CANCER INSTITUTE, East Melbourne (AU)

(72) Inventors: James E. Bradner, Weston, MA (US); Simon John Hogg, Carlton (AU); Ricky Wayne Johnstone, Alphington (AU); Jake Shortt, Northcote (AU)

(21) Appl. No.: 15/764,131

(22) PCT Filed: Sep. 30, 2016
(86) PCT No.: PCT/US16/54924

§ 371 Date: Mar. 28, 2018

Related U.S. Application Data
(60) Provisional application No. 62/236,280, filed on Oct. 2, 2015.

(51) Int. Cl. A61K 31/551; A61K 39/395; C07K 16/28; A61P 35/00

(52) U.S. Cl. CPC A61K 31/551; A61K 39/3955; C07K 16/2827; A61K 2039/505; A61P 35/00; C07K 2317/24; C07K 16/2878

(57) ABSTRACT

The present disclosure provides combination therapy of a bromodomain inhibitor and an immune modulator (e.g., an immune check point inhibitor). The combination of the bromodomain inhibitor and the immune modulator may be useful in treating or preventing cancer in a subject. In certain embodiments, the subject has an intact immune system. The combination of the bromodomain inhibitor and the immune modulator is expected to be synergistic.

### RELATED APPLICATION

This application claims the benefit of U.S. Provisional Application No. 62/236,280, filed on Oct. 2, 2015. The entire teachings of the above application is incorporated herein by reference.

### BACKGROUND OF THE INVENTION

Bromodomain-containing proteins are of substantial biological interest, as components of transcription factor complexes and determinants of epigenetic memory. For example, the bromo and extra terminal (BET) protein family (e.g., bromodomain-containing protein 2 (BRD2), bromodomain-containing protein 3 (BRD3), bromodomain-containing protein 4 (BRD4), and bromodomain testis-specific protein (BRDT)) shares a common domain architecture featuring two amino-terminal bromodomains that exhibit high levels of sequence conservation, and a more divergent carboxy-terminal recruitment domain (Filippakopoulos et al., Nature 2010, 468, 1067-1073). BRD2 and BRD3 are reported to associate with histones along actively transcribed genes and may be involved in facilitating transcriptional elongation (Leroy et al., Mol. Cell. 2008, 30, 51-60). It has also been reported that BRD4 or BRD3 may fuse with nuclear protein in testis (NUT), forming novel fusion oncogenes BRD4-NUT or BRD3-NUT, in a highly malignant form of epithelial neoplasia (French et al., Cancer Res., 2003, 63, 304-307; French et al., J. Clin. Oncol. 2004, 22, 4135-4139). Data suggests that BRD-NUT fusion proteins contribute to carcinogenesis (French et al., Oncogene 2008, 27, 2237-2242). BRDT is uniquely expressed in the testes and ovary. All family members of BET have been reported to have some function in controlling or executing aspects of the cell cycle and have been shown to remain in complex with chromosomes during cell division, suggesting a role in the maintenance of epigenetic memory. In addition, some viruses make use of BET proteins to tether their genomes to the host cell chromatin, as part of the process of viral replication (You et al., Cell 2004, 117, 349-360). BRD4 appears to be involved in the recruitment of the pTEF-b complex to inducible genes, resulting in phosphorylation of RNA polymerase and increased transcriptional output (Hargreaves et al., Cell 2009, 138,129-145). In humans, BRD2, BRD3, BRD4, and BRDT exhibit similar gene arrangements, domain organizations, and some functional properties (Wu et al., J. Biol. Chem. 2007, 282, 13141-13145). Modulation of bromo-domain containing proteins (e.g., BET proteins) may be useful in treating a variety of conditions for example, in treating cancer by altering epigenetic expression of certain genes in cancer cells.

### SUMMARY OF THE INVENTION

The present invention is based, at least in part, on the surprising discovery that combinations of certain bromodomain inhibitors and certain immune modulators (e.g., immune checkpoint inhibitors) are particularly effective at treating subjects having cancer (e.g. hematological cancers or solid organ tumors). Thus, the present disclosure relates to improved methods of treating cancer.

In some aspects, the disclosure provides a method of treating cancer in a subject in need thereof, the method comprising administering to the subject a therapeutically effective amount of a bromodomain inhibitor; and, an immune modulator (e.g., immune checkpoint inhibitor).

Aspects of the invention relate to the surprising discovery that bromodomain inhibitors require an intact immune system for optimal efficacy in treatment of cancer. Thus, in some embodiments, the subject has an intact immune system. In some embodiments, the subject is a human.

In some embodiments, the bromodomain inhibitor and the immune modulator (e.g., immune checkpoint inhibitor) are synergistic in treating the cancer, compared to the bromodomain inhibitor alone or the immune modulator (e.g., immune checkpoint inhibitor alone.

In some embodiments, the cancer is a hematological cancer or a solid organ tumor. In some embodiments, the hematological cancer is lymphoma, leukemia, or myeloma. In some embodiments, the solid organ tumor is a liver, colon, breast, lung, prostate, kidney, head and neck, melanoma, skin, pancreas, or brain tumor.

In some embodiments, the bromodomain inhibitor is a peptide, antibody, interfering RNA, or small molecule. In some embodiments, the bromodomain inhibitor is a small molecule.

The bromodomain inhibitor useful in the methods of the present disclosure may be any bromodomain inhibitor known in the art or developed in the future. In certain embodiments, the bromodomain inhibitor is a compound of Formulae (I)-(XI):

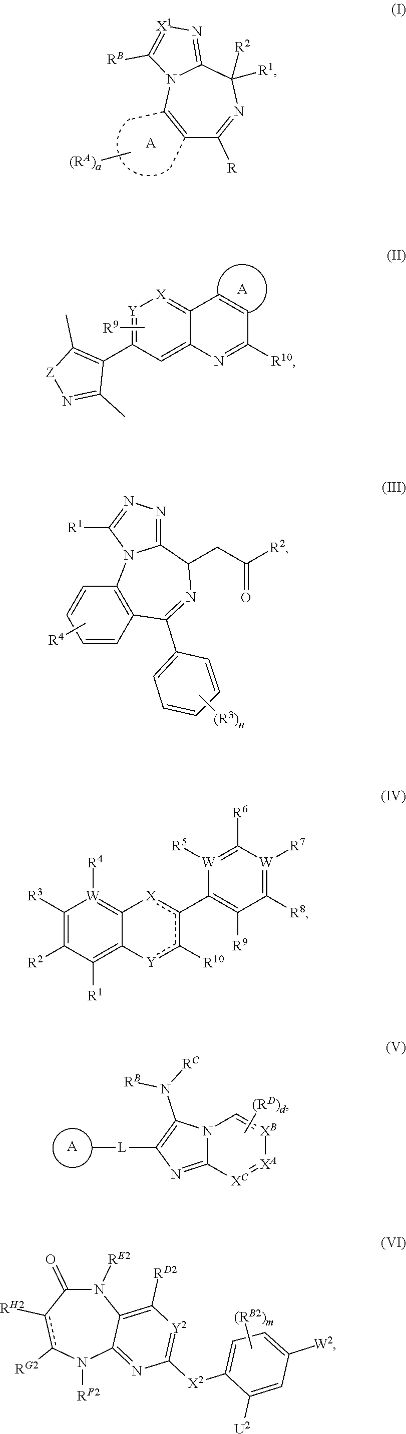

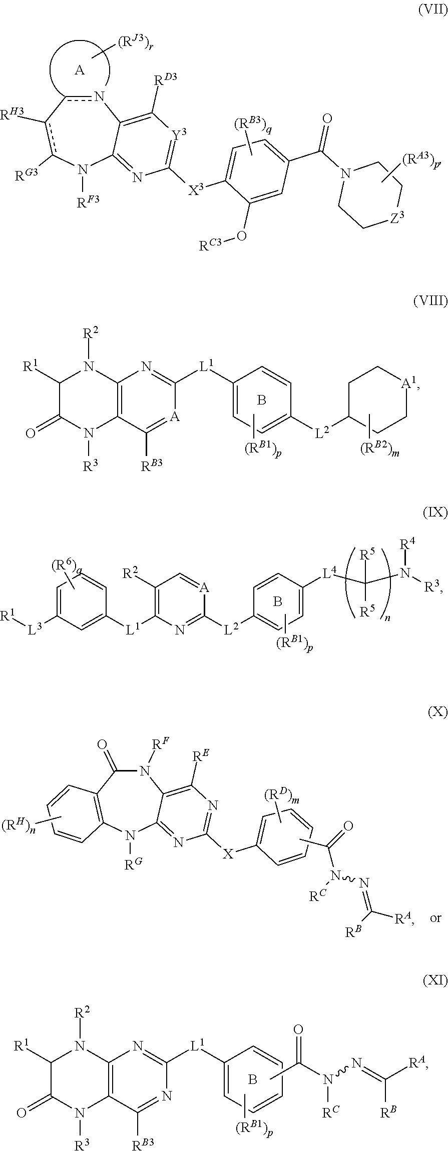

or a pharmaceutically acceptable salt, solvate, hydrate, polymorph, co-crystal, tautomer, stereoisomer, isotopically labeled derivative, or prodrug thereof.

In some embodiments, the bromodomain inhibitor is not of Formula

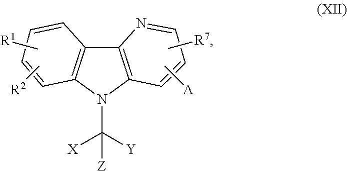

or a pharmaceutically acceptable salt, solvate, hydrate, polymorph, co-crystal, tautomer, stereoisomer, isotopically labeled derivative, or prodrug thereof. 100111 In some embodiments, the bromodomain inhibitor of Formula (I) is a bromodomain inhibitor having a Formula selected from the group consisting of: I-A, I-B, I-C, I-D, I-E, I-F, I-G, I-H, I-J, I-K, I-L, I-M, I-N, I-O, I-P, I-Q, and I-R.

In some embodiments, the bromodomain inhibitor of Formula (II) is a bromodomain inhibitor having a Formula selected from the group consisting of: II-A, II-B, II-C, II-D, II-E, and II-F.

In some embodiments, the bromodomain inhibitor of Formula (III) is a bromodomain inhibitor having a Formula selected from the group consisting of: III-A, III-B, III-C, III-D, and III-E.

In some embodiments, the bromodomain inhibitor of Formula (IV) is a bromodomain inhibitor having a Formula selected from the group consisting of: IV-A and IV-B.

In some embodiments, the bromodomain inhibitor of Formula (V) is a bromodomain inhibitor having a Formula selected from the group consisting of V-A, V-B, V-C, V-D, V-E, V-F, V-G, V-H, and V-J.

In some embodiments, the bromodomain inhibitor of Formula (VI) is a bromodomain inhibitor having a Formula selected from the group consisting of: VI-A, VI-B, VI-C, and VI-D.

In some embodiments, the bromodomain inhibitor of Formula (VII) is a bromodomain inhibitor having a Formula selected from the group consisting of: VII-A, VII-B, and VII-C.

In some embodiments, the bromodomain inhibitor of Formula (VIII) is a bromodomain inhibitor having a Formula selected from the group consisting of: VIII-A, VIII-B, VIII-C, and VIII-D.

In some embodiments, the bromodomain inhibitor of Formula (IX) is a bromodomain inhibitor having a Formula selected from the group consisting of: IX-A, IX-B, IX-C, IX-D, IX-E, IX-F, and IX-G.

In some embodiments, the bromodomain inhibitor is JQ1. In some embodiments, the bromodomain inhibitor is IBET-151. In some embodiments, the bromodomain inhibitor is IBET-762. In some embodiments, the bromodomain inhibitor is RVX-208. In some embodiments, the bromodomain inhibitor is Y803 (OTX-15). In some embodiments, the bromodomain inhibitor is dBET1. In some embodiments, the bromodomain inhibitor is CPI-203.

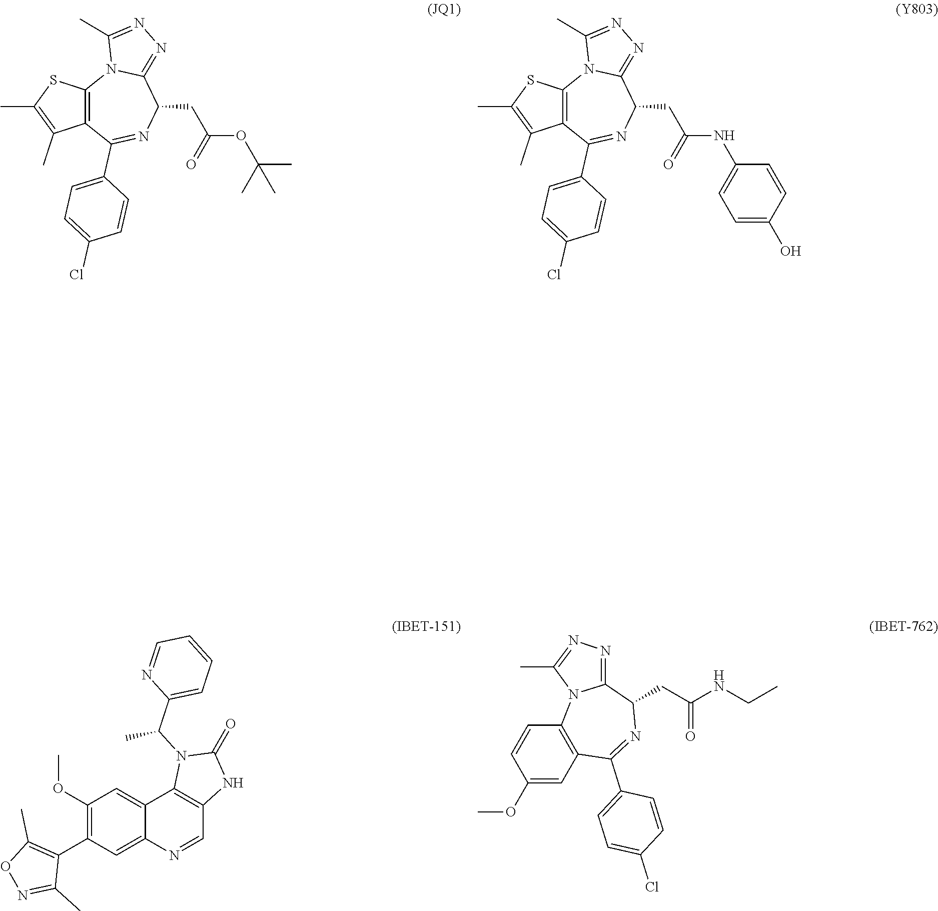 [SMILES: Cc1sc2c(c1C)C(c1ccc(Cl)cc1)=N[C@@H](CC(=O)OC(C)(C)C)c1nnc(C)n1-2 ; Cc1sc2c(c1C)C(c1ccc(Cl)cc1)=N[C@@H](CC(=O)Nc1ccc(O)cc1)c1nnc(C)n1-2 ; COc1cc2c(cc1-c1c(C)noc1C)ncc1[nH]c(=O)n([C@H](C)c3ccccn3)c12 ; CCNC(=O)C[C@@H]1N=C(c2ccc(Cl)cc2)c2cc(OC)ccc2-n2c(C)nnc21]

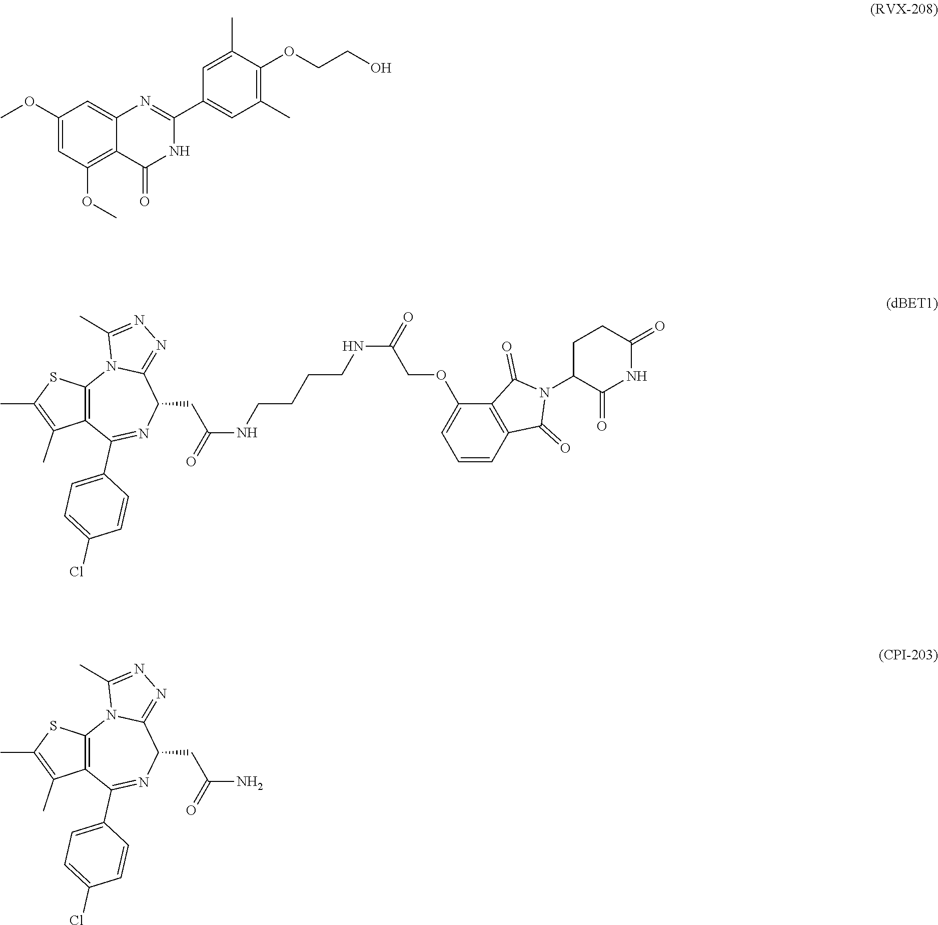 [SMILES: COc1cc(OC)c2c(=O)[nH]c(-c3cc(C)c(OCCO)c(C)c3)nc2c1 ; Cc1sc2c(c1C)C(c1ccc(Cl)cc1)=N[C@@H](CC(=O)NCCCCNC(=O)COc1cccc3c1C(=O)N(C1CCC(=O)NC1=O)C3=O)c1nnc(C)n1-2 ; Cc1sc2c(c1C)C(c1ccc(Cl)cc1)=N[C@@H](CC(N)=O)c1nnc(C)n1-2]

In some embodiments, the bromodomain inhibitor of Formula (I) is a bromodomain inhibitor having a Formula selected from the group consisting of: I-A, I-B, I-C, I-D, I-E, I-F, I-G, I-H, I-J, I-K, I-L, I-M, I-N, I-O, I-P, I-Q, and I-R.

In some embodiments, the bromodomain inhibitor of Formula (II) is a bromodomain inhibitor having a Formula selected from the group consisting of: II-A, II-B, II-C, II-D, II-E, and II-F.

In some embodiments, the bromodomain inhibitor of Formula (III) is a bromodomain inhibitor having a Formula selected from the group consisting of: III-A, III-B, III-C, III-D, and III-E.

In some embodiments, the bromodomain inhibitor of Formula (IV) is a bromodomain inhibitor having a Formula selected from the group consisting of: IV-A and IV-B.

In some embodiments, the bromodomain inhibitor of Formula (V) is a bromodomain inhibitor having a Formula selected from the group consisting of: V-A, V-B, V-C, V-D, V-E, V-F, V-G, V-H, and V-J.

In some embodiments, the bromodomain inhibitor of Formula (VI) is a bromodomain inhibitor having a Formula selected from the group consisting of: VI-A, VI-B, VI-C, and VI-D.

In some embodiments, the bromodomain inhibitor of Formula (VII) is a bromodomain inhibitor having a Formula selected from the group consisting of: VII-A, VII-B, and VII-C.

In some embodiments, the bromodomain inhibitor of Formula (VIII) is a bromodomain inhibitor having a Formula selected from the group consisting of: VIII-A, VIII-B, VIII-C, and VIII-D.

In some embodiments, the bromodomain inhibitor of Formula (IX) is a bromodomain inhibitor having a Formula selected from the group consisting of IX-A, IX-B, IX-C, IX-D, IX-E, IX-F, and IX-G.

In some embodiments, the immune modulator activates expression or activity of a stimulatory immune molecule. In some embodiments, the stimulatory immune molecule is selected from the group consisting of 4-1BB (CD137), CD137L, OX40, OX40L, ICOS, CD40, CD40L, CD70, CD27, CD28, CD80, CD86, B7RP1, and HVEM. In some embodiments, the immune modulator inhibits expression or activity of an inhibitory immune molecule (e.g., an immune checkpoint molecule). In some embodiments, the immune modulator is an immune checkpoint inhibitor. In some embodiments, the immune checkpoint inhibitor is an inhibitor of an immune checkpoint protein selected from the group consisting of: CTLA-4, PD-1, PDL-1, PDL-2, TIM3, LAG3, B7-H3, B7-H4, BTLA, GAL9, and A2aR.

In some embodiments, the immune modulator is a peptide, antibody, interfering RNA, or small molecule. In some embodiments, the immune modulator is a monoclonal antibody, or an Ig fusion protein. In some embodiments, the immune modulator is an agonistic antibody directed to a stimulatory immune molecule (e.g., 4-1BB (CD137), CD137L, OX40, OX40L, ICOS, CD40, CD40L, CD70, CD27, CD28, CD80, CD86, B7RP1, or HVEM).

In some embodiments, the immune modulator is an immune checkpoint inhibitor. In some embodiments, the immune checkpoint inhibitor is a peptide, antibody, interfering RNA, or small molecule. In some embodiments, the immune checkpoint inhibitor is a monoclonal antibody, or an Ig fusion protein. In some embodiments, the immune checkpoint inhibitor is an inhibitor of an immune checkpoint protein selected from the group consisting of: CTLA-4, PD-1, PDL-1, PDL-2, TIM3, LAG-3, B7-H3, B7-H4, BTLA, GAL9, and A2aR.

In some embodiments, the bromodomain inhibitor and the immune modulator (e.g., immune checkpoint inhibitor) are administered to the subject simultaneously as a single composition. In some embodiments, the bromodomain inhibitor and the immune modulator (e.g., immune checkpoint inhibitor) are administered to the subject separately. In some embodiments, the bromodomain inhibitor and the immune modulator (e.g., immune checkpoint inhibitor) are administered to the subject concurrently (e.g., administered at the same time as separate compositions). In some embodiments, the bromodomain inhibitor is administered to the subject after the immune modulator (e.g., immune checkpoint inhibitor).

In some embodiments, the bromodomain inhibitor is administered to the subject prior to the immune modulator. In some embodiments, the administration of the bromodomain inhibitor occurs at least 24 hours (1 day), 2 days, 3 days or 4 days prior to the administration of the immune modulator. In some embodiments, the bromodomain inhibitor and the immune modulator (e.g., immune checkpoint inhibitor) co-administered (e.g., simultaneously or concurrently administered) to the subject.

Other advantages, features, and uses of the invention will be apparent from the detailed description of certain non-limiting embodiments; the drawings, which are schematic and not intended to be drawn to scale; and the claims.

### BRIEF DESCRIPTION OF THE DRAWINGS

FIGS. 1A-1D show data demonstrating that an intact host immune system is required for the robust anti-cancer effects of JQ1 against a murine model of aggressive B-cell lymphoma. FIGS. 1A-1B show Kaplan-Meier survival curves representing cohorts of wild type C57BL/6 mice and immune compromised strains; FIG. 1A shows C57BL/6.Rag2cγ^{-/-} mice inoculated with Eμ-Myc lymphoma ^{#}4242 and treated with JQ1 (solid line), or DMSO vehicle (dashed line); FIG. 1B shows C57BL/6.Rag1^{-/-} inoculated with Eμ-Myc lymphoma ^{#}4242 and treated with JQ1 (solid line), or DMSO vehicle (dashed line); FIG. 1C shows Kaplan-Meier survival curves representing cohorts of wild type C57BL/6 mice and immune compromised strain C57BL/6.Rag2cγ^{-/-} inoculated with Eμ-Myc lymphoma ^{#}299 and treated with JQ1 (solid line), or DMSO vehicle (dashed line); FIG. 1D shows a representative flow cytometry histogram demonstrating that splenic front tumor hearing mice express high levels of PD-1, indicative of an exhausted phenotype. (*p<0.05, **p<0.01, “*p<0.001, Log-rank).

FIGS. 2A-2I show PD-L1 is a direct target of BET inhibition in vitro and in vivo. FIGS. 2A-2B show JQ1 downregulates the expression of PD-L1 (CD274) on lymphoma cells by flow cytometry; FIG. 2A shows a graph of mean florescence intensity (MFI) on Eμ-Myc lymphoma cell line ^{#}4242; FIG. 2B shows a graph of mean florescence intensity (MFI) on Eμ-Myc lymphoma cell line ^{#}299; both cell lines over-express Bcl-2 and were measured following 24 hours treatment in vitro with indicated concentrations of JQ1, or DMSO control. Representative data is presented as mean MFI of cells cultured and analyzed in triplicate±S.E.M. (****p<0.0001, Student's t test); FIG. 2C shows representative histograms demonstrating that PD-L1 downregulation following BET inhibition is time-dependent; FIG. 2D shows a graph of the MFI of PD-L1 expression gated on live GFP-positive tumor cells; FIG. 2E shows a graph of the MFI of PD-L2 expression gated on live GFP-positive tumor cells; FIG. 2F shows circulating tumor cells from the peripheral blood of C57BL/6 mice bearing Eμ-Myc lymphoma and treated chronically with JQ1 express lower levels of PD-L1; FIG. 2G shows quantitative real-time-PCR (qPCR) analysis of PD-L1 mRNA levels in Eμ-Myc lymphoma cell line ^{#}4242, FIG. 2H shows quantitative real-time-PCR (qPCR) analysis of PD-L1 mRNA levels in Eμ-Myc lymphoma cell line ^{#}299; both cell lines overexpress Bcl-2 and were measured following treatment with 1000 nM JQ1, or DMSO control, for indicated time points; FIG. 2I shows chromatin immunoprecipitation-PCR of Eμ-Myc lymphoma ^{#}299 showing binding of BRD4 at the PD-L1 locus following 2 hours treatment in vitro with 1000 nM JQ1, or DMSO control.

FIGS. 3A-3E show genetic knockdown of BRD4 phenocopies BET inhibitor treatment. FIG. 3A shows representative FACS plots of ^{#}4242 expressing sh.BRD4.498, sh.BRD4.500, and sh.SCR treated in the presence of absence of Dox for 16 hours in vitro; FIG. 3B shows a graph of MFI of PD-L1 expression on GFP⁺DsRed⁺ populations following 16 hours in vitro treatment with Dox. Representative data is presented as mean MFI of cells cultured and analyzed in triplicate±S.E.M (*p<0.05, **p<0.01, Student's t test); FIG. 3C shows MFI of PD-L1 expression on Hodgkin lymphoma cell line L540 after treatment for 24 hours in vitro with indicated concentrations of JQ1; FIG. 3D shows MFI of PD-L1 expression and IFN-γ-mediated induction of PD-L1 that can be abrogated with the co-treatment of JQ1; FIG. 3E shows MFI of PD-L1 on Eμ-Myc lymphoma cell line ^{#}6066 following 24 hours treatment in vitro with 1 μM JQ1, IBET-151, IBET-762, Y803, or dBET1, 10 μM RVX-208, or DMSO control. Representative data is presented as mean MFI of cells cultured and analyzed in triplicate±S.E.M. (***p<0.001, Student's t test).

FIGS. 4A-4B show JQ1 in combination with checkpoint inhibitors or immune stimulating antibodies promotes curative anti-tumor responses. FIGS. 4A-4B show Kaplan-Meier survival curves representing cohorts of C56BL/6 (n=6 per treatment group) injected intravenously with 1-5×10⁵ Eμ-Myc lymphoma ^{#}299 cells; FIG. 4A shows the efficacy of JQ1 in combination with PD-1 blockade against Eμ-Myc lymphoma ^{#}299; FIG. 4B shows the efficacy of JQ1 in combination with the agonistic anti-4-1BB (CD137) immune stimulating antibody against Eμ-Myc lymphoma ^{#}299.

### DEFINITIONS

### Chemical Terms

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York. 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, ——— is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

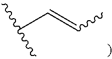

may be an (E)- or (Z)- double bond.

The term “heteroalkenyl” refers to an alkenyl group, which further includes at least one heteroatom 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”), in some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkenyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more acyl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”), in some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen., and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom e.g., indolyl, quinolinyl carbazolyl, and the like) the point of attachment can be on either ring, i.e., either the ring bearing a heteroatom (e.g., 2-indolyl) or the ring that does not contain a heteroatom e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl heterocyclyl aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₁₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, amino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}), and amides (—C(═O)N(R^{bb})₂, —C(═O)NR^{bb}SO₂R^{aa}, —C(═S)N(R^{bb})₂), wherein R^{aa} and R^{bb} are as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(NR^{cc})OR^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC-₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a nitrogen protecting group described herein is Bn, Boc, Cbz, Fmoc, trifluoroacetyl, triphenylmethyl, acetyl, tosyl, nosyl, brosyl, mesyl, or triflyl.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetimide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc) 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(trihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl, carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide triphenylmethylsulfenamide and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Nuts, 3^{rd} edition, John Wiley & Sons, 1999, incorporated herein by reference. In certain embodiments, an oxygen protecting group described herein is silyl, TBDPS, TBDMS, TIPS, TES, TMS, MOM, THP, t-Bu, Bn, allyl, acetyl, pivaloyl, or benzoyl.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzafuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsiyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC). 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphorsulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not intended to be limited in any manner by the above exemplary listing of substituents.

### Other Definitions

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or maionic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may he prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.xH₂O, wherein R is the compound, and x is a number greater than 0. A given compound may form more than one type of hydrate, including, e.g., monohydrates (x is 1), lower hydrates (x is a number greater than 0 and smaller than 1, hemihydrates (R.0.5.H₂O)), and polyhydrates (x is a number greater than 1, e.g., dihydrates (R.2H₂O) and hexahydrates (R.6H₂O)).

The term “tautomers” or “tautomeric” refers to two or more interconvertible compounds resulting from at least one formal migration of a hydrogen atom and at least one change in valency a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents, in certain embodiments, the small molecule is a drug. Preferably, though not necessarily, the drug is one that has already been deemed sale and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “inhibition”, “inhibiting”, “inhibit,” or “inhibitor” refer to the ability of a compound to reduce, slow, halt, and/or prevent activity of a particular biological process in a cell relative to vehicle.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments the non-human animal is a fish, reptile, or amphibian. The non-human animal may be a male or female at any stage of development. The non-human animal may be a transgenic animal or genetically engineered animal. A “patient” refers to a human subject in need of treatment of a disease.

The terms “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay and/or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, hematological malignancies. The term “hematological malignancy” refers to tumors that affect blood, bone marrow, and/or lymph nodes. Exemplary hematological malignancies include, but are not limited to, leukemia, such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma, such as Hodgkin lymphoma (HL) (e.g., B-cell HL, T-cell HL) and non-Hodgkin lymphoma (NHL) (e.g., B-cell NHL, such as diffuse large cell lymphoma (DLCL) (e.g., diffuse large B-cell lymphoma (DLBCL, e.g., activated B-cell (ABC) DLBCL (ABC-DLBCL))), follicular lymphoma, chronic lymphocytic leukemia/small lymphocytic lymphoma (CLL/SLL), mantle cell lymphoma (MCL), marginal zone B-cell lymphoma (e.g., mucosa-associated lymphoid tissue (MALT) lymphoma, nodal marginal zone B-cell lymphoma, splenic marginal zone B-cell lymphoma), primary mediastinal B-cell lymphoma, Burkitt lymphoma, Waldenström's macroglobulinemia (WM, lymphoplasmacytic lymphoma), hairy cell leukemia (HCL), immunoblastic large cell lymphoma, precursor B-lymphoblastic lymphoma, central nervous system (CNS) lymphoma (e.g., primary CNS lymphoma and secondary CNS lymphoma); and T-cell NHL, such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma), lymphoma of an immune privileged site (e.g., cerebral lymphoma, ocular lymphoma, lymphoma of the placenta, lymphoma of the fetus, testicular lymphoma); a mixture of one or more leukemia/lymphoma as described above; myelodysplasia; and multiple myeloma (MM). Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma), bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma), familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasophangeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS), mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor arid not of that of the organ or tissue in which the secondary (metastatic) tumor is located. For example, a prostate cancer that has migrated to bone is said to be metastasized prostate cancer and includes cancerous prostate cancer cells growing in bone tissue.

### DETAILED DESCRIPTION OF CERTAIN EMBODIMENTS OF THE INVENTION

Aspects of the disclosure relate to the surprising discovery that certain combinations of bromodomain inhibitors and immune modulators (e.g., immune checkpoint inhibitors) are particularly effective in treating some types of cancers (e.g., hematological cancers and solid organ tumors). The invention is based, at least in part, on the recognition that administration of bromodomain inhibitors synergistically enhances the anti-cancer effects of immune checkpoint inhibitors.

### Methods of Treating Cancer

In some aspects, the disclosure provides a method of treating cancer in a subject in need thereof, the method comprising administering to the subject a therapeutically effective amount of a bromodomain inhibitor; and, an immune checkpoint inhibitor.

As used herein, “a subject in need thereof” is a subject having, or suspected of having cancer, e.g., the subject has been diagnosed by a physician (e.g., using methods well known in the art; see, for example, Methods of Cancer Diagnosis, Theapy and Prognosis, Hayat (Ed.), vols. 1-8, 2008-2010). Examples of methods for diagnosing cancer include, but are not limited to blood tests, urine tests, tissue biopsy, image-based tests (e.g., magnetic resonance imaging (MRI), computerized tomography (CT scans), and x-ray), and molecular tests (e.g., PCR-based diagnostic methods).

Aspects of the disclosure relate to the surprising discovery that bromodomain inhibitors require an intact immune system for optimal efficacy in treatment of cancer. As used herein, the term “intact immune system” refers to subject (e.g., a human) with a functional immune system capable of raising an immune response to a foreign antigen. Thus, a subject having an “intact immune system” has a full complement of immune effector cells (e.g., T-cells, B-cells, NK cells, dendritic cells, myeloid cells) and immune effector molecules (e.g., perforin, granzymes, death receptors, T-cell receptors, co-stimulatory molecules). The immune response includes, for example, the ability to generate B cells that secrete antibodies.

Aspects of the invention relate to use of a combination of a bromodomain inhibitor and an immune checkpoint inhibitor for the treatment of a hematological cancer and/or a solid organ tumor. In some embodiments the hematological cancer is lymphoma, leukemia, or myeloma. Examples of hematological cancers include, but are not limited to acute lymphocytic leukemia (ALL), acute myelocytic leukemia (AML), chronic myelocytic leukemia (CML), Hodgkin lymphoma (HL), non-Hodgkin lymphoma (NHL), mantle cell lymphoma (MCL), B-cell lymphoma, and multiple myeloma. In some embodiments, the cancer is a solid organ tumor. Examples of solid organ tumors include, but are not limited to, tumors of the liver, colon, breast, lung, prostate, brain, kidney, head and neck, melanoma, skin, pancreas, colorectum, bladder, sarcoma (e.g., tumors of bone or muscle), and melanocytes (e.g., melanoma).

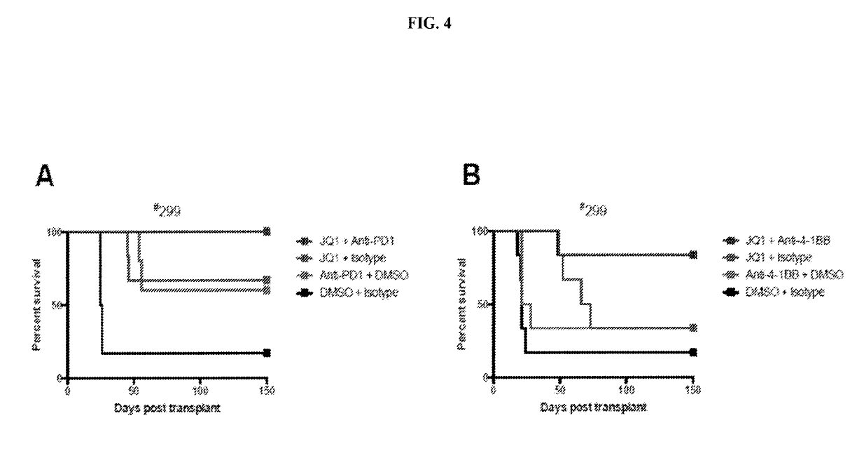

Aspects of the invention relate to the discovery that certain combinations of bromodomain inhibitors and immune checkpoint inhibitors exhibit synergistic anti-cancer effects when administered to a subject having or suspected of having cancer. As used herein, the terms “synergistically” or “synergy” refer to refers to the joint action of agents (e.g., pharmaceutically active agents), that when taken together increase each other's effectiveness. Without wishing to be bound by any particular theory, certain bromodomain inhibitors (e.g., JQ1) down-regulate immune checkpoint proteins (e.g., PD-L1) and increase the therapeutic efficacy of immune checkpoint inhibitors (e.g., anti-PD-L1 antibody) compared to treatment with the bromodomain inhibitor or the immune checkpoint inhibitor alone. The synergistic effects of bromodomain inhibitor/immune checkpoint inhibitor combinations are described in the Examples section and in FIG. 4.

Assessment of therapeutic efficacy can be performed by any suitable method in the art. For example, therapeutic efficacy in treating a solid tumor can be assessed by measurement of tumor growth (e.g., inhibition of tumor growth), or a reduction in tumor size. In another example, therapeutic efficacy in treating a hematological cancer can be assessed by measuring induction of apoptosis in cancer cells (e.g., by annexin V staining) that have been treated with the combination of a bromodomain inhibitor and an immune checkpoint inhibitor. Additional methods of assessing therapeutic efficacy of cancer treatments are disclosed, for example, in Textbook of Medical Oncology 4^{th}Ed., Cavalli et al. (Eds.), Taylor & Francis, 2009 and in Cell Death Techniques—A Laboratory Manual, Johnstone and Silke (Eds.), Cold Spring Harbor Press, 2015.

A bromodomain inhibitor can be a peptide, antibody, interfering RNA, or small molecule. Examples of antisense compounds include, but are not limited to interfering RNAs (e.g., dsRNA, siRNA, shRNA, miRNA, and amiRNA), antisense oligonucleotides (ASO), and aptamers (e.g., DNA aptamers and RNA aptamers). In some embodiments, a bromodomain inhibitor is a small molecule.

In some embodiments, the bromodomain inhibitor is a bromodomain inhibitor selected from the group consisting of formulas (I)-(XI), or a pharmaceutically acceptable salt, solvate, hydrate, polymorph, co-crystal, tautomer, stereoisomer, isotopically labeled derivative, or prodrug thereof. Such bromodomain inhibitors are described in further detail below.

### Bromodomain Inhibitors

In some aspects, the invention relates to the surprising discovery that combinations of certain bromodomain inhibitors and certain immune checkpoint inhibitors are particularly effective at treating subjects having cancer.

The term bromodomain inhibitors refers to an inhibitor of a bromodomain or an inhibitor of a bromodomain-containing protein. In certain embodiments, the bromodomain inhibitor is an inhibitor of a bromodomain and extra-terminal (BET) protein. In certain embodiments, the bromodomain inhibitor is an inhibitor of bromodomain-containing protein 2 (BRD2), bromodomain-containing protein 2 (BRD2), bromodomain-containing protein 2 (BRD2), or bromodomain-containing protein 2 (BRD2). In certain embodiments, the bromodomain inhibitor is an inhibitor of a (TATA box binding-protein)-associated factor (TAF) protein (e.g., TAF1 or TAF1L). In certain embodiments, the bromodomain inhibitor is an inhibitor of CREB binding protein (CBP). In certain embodiments, the bromodomain inhibitor is an inhibitor of E1 A binding protein p300 (EP300).

In some embodiments, the bromodomain inhibitor is not of Formula (XII):

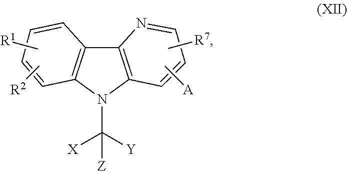

or a pharmaceutically acceptable salt, solvate, hydrate, polymorph, co-crystal, tautomer, stereoisomer, isotopically labeled derivative, or prodrug thereof.

### Compounds of Formula (I)

In certain embodiments, the bromodomain inhibitor is an inhibitor disclosed in international PCT Publication No. WO 2011/143669; U.S. Pat. No. 8,981,083; U.S. Patent Publication No. US 2013/0184264; or U.S. Patent Publication No. US 2015/0150885, each of which is incorporated herein by reference.

In certain embodiments, the bromodomain inhibitor is an inhibitor disclosed in international PCT Publication No. WO 2009/084693; international PCT Publication No. WO 2006/310709; U.S. Pat. No. 8,476,260; U.S. Pat. No. 8,044,042; U.S. Pat. No. 5,712,274; U.S. Patent Publication No. US 2010/0286127; U.S. Patent Publication No. US 2013/0261109; or U.S. Patent Publication No. US 2010/0041643, each of which is incorporated herein by reference.

In certain embodiments, the bromodomain inhibitor is of Formula (I):

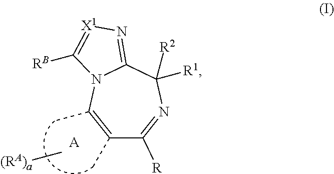

or pharmaceutically acceptable salt, solvate, hydrate, polymorph, co-crystal, tautomer, stereoisomer, isotopically labeled derivative, or prodrug thereof, wherein:
X¹ is N or CR⁵;
R⁵ is hydrogen, alkyl, carbocyclyl, heterocyclyl, aryl, or heteroaryl, each of which is optionally substituted;
R^{B} is hydrogen, alkyl, hydroxylalkyl, aminoalkyl, alkoxyalkyl, haloalkyl, hydroxy, alkoxy, or —C(═O)O—R³, each of which is optionally substituted;
Ring A is aryl or heteroaryl;
each R^{A} is independently alkyl, carbocyclyl, heterocyclyl, aryl, or heteroaryl, each of which is optionally substituted; or two R^{A} attached to adjacent atoms are joined to form an optionally substituted aryl or optionally substituted heteroaryl ring;
R is alkyl, cycloalkyl, heterocycloalkyl, aryl, or heteroaryl, each of which is optionally substituted;
R¹ is —(CH₂)ₙ-L, wherein n is 0, 1, 2, or 3, and L is hydrogen, —C(═O)O—R³, —C(═O)—R³, —C(═O)—N(R³R⁴), —S(═O)₂—R³, —S(═O)₂N(R³R⁴), —N(R³R⁴), —N(R⁴)C(═O)R³, optionally substituted aryl, or optionally substituted heteroaryl;
R² is hydrogen, halogen, or optionally substituted alkyl;
each R³ is independently hydrogen., optionally substituted alkyl optionally substituted alkenyl, optionally substituted alkynyl, optionally substituted aryl, substituted aryl, heteroaryl, optionally substituted heterocyclyl, optionally substituted carbocyclyl, —NH₂, or —N═CR⁴R⁶;
each occurrence of R₄ is independently hydrogen, optionally substituted alkyl, optionally substituted alkenyl, optionally substituted alkynyl, optionally substituted aryl, substituted aryl, heteroaryl, optionally substituted heterocyclyl, optionally substituted carbocyclyl, —NH₂, or —N═CR⁴R⁶;
or R³ and R⁴ are taken together with the nitrogen atom to which they are attached to form an optionally substituted heterocyclyl or optionally substituted heteroaryl ring;
R⁶ is alkyl, alkenyl, carbocyclyl, heterocyclyl, heterocycloalkyl, aryl, or heteroaryl, each of which is optionally substituted;
or R⁴ and R⁶ are taken together with the carbon atom to which they are attached to form a an optionally substituted heterocyclyl ring; and
a is 0,1, 2, or 3.

In certain embodiments, the bromodomain inhibitor of Formula (I) is of Formula (I-A):

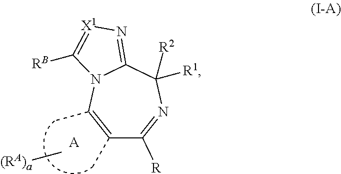

or pharmaceutically acceptable salt, solvate, hydrate, polymorph, co-crystal, tautomer, stereoisomer, isotopically labeled derivative, or prodrug thereof, wherein:
X¹ is N or CR⁵;
R⁵ is hydrogen, alkyl, carbocyclyl, heterocyclyl, aryl, or heteroaryl, each of which is optionally substituted;
R^{B} is hydrogen, alkyl, hydroxylalkyl, aminoalkyl, alkoxyalkyl, haloalkyl, hydroxy, alkoxy, or —C(═O)O—R³, each of which is optionally substituted;
Ring A is aryl or heteroaryl;
each R^{A} is independently alkyl, carbocyclyl, heterocyclyl, aryl, or heteroaryl, each of which is optionally substituted; or two R^{A} attached to adjacent atoms are joined to form an optionally substituted aryl or optionally substituted heteroaryl ring;
R is alkyl, carbocyclyl, heterocyclyl, aryl, or heteroaryl, each of which is optionally substituted;
R¹ is —(CH₂)ₙ-L, wherein n is 0, 1, 2, or 3, and L is hydrogen, —C(═O)O—R³, —C(═O)—R³, —C(═O)—N(R³R⁴), —S(═O)₂—R³, —S(═O)₂—N(R³R⁴), —N(R³R⁴), —N(R⁴)C(═O)R³, optionally substituted aryl, or optionally substituted heteroaryl;
R² is hydrogen, halogen, or optionally substituted alkyl;
each R³ is independently selected from the group consisting of:
   (i) hydrogen, aryl, substituted aryl, heteroaryl, or substituted heteroaryl;
   (ii) heterocyclyl or substituted heterocyclyl;
   (iii) C₁₋₈ alkyl, C₂₋₈ alkenyl, or C₂₋₈ alkynyl, each of which contains 0, 1, 2, or 3 heteroatoms selected from O, S, and N, or C₃₋₁₂ carbocyclyl, each of which is optionally substituted; and
   (iv) —NH₂ or —N═CR⁴R⁶;
each R₄ is independently hydrogen, alkyl, alkyl, carbocyclyl, heterocyclyl, aryl, or heteroaryl, each of which is optionally substituted;
or R³ and R⁴ are taken together with the nitrogen atom to which they are attached to form a 4- to 10-membered ring; and
R⁶ is alkyl, alkenyl, carbocyclyl, heterocyclyl, aryl, or heteroaryl, each of which is optionally substituted;
or R⁴ and R⁶ are taken together with the carbon atom to which they are attached to form a 4- to 10-membered ring;
a is 0, 1, 2, or 3;
   provided that:
(a) if Ring A is thienyl, X¹ is N, R is phenyl or substituted phenyl R² is hydrogen, R^{B} is methyl, R¹ is —(CH₂)ₙ-L, n is 1, and L is —C(═O)—N(R³R⁴), then R₃ and R₄ are not taken together with the nitrogen atom to which they are attached to form a morpholino ring;
(b) if Ring A is thienyl, X¹ is N, R is substituted phenyl, R² is hydrogen, R^{B} is methyl, R¹ is —(CH₂)ₙ-L, n is 1, L is —C(═O)—N(R³R⁴), and one of R₃ and R₄ is hydrogen, then the other of R³ and R⁴ is not methyl, hydroxyethyl, alkoxy, phenyl, substituted phenyl, pyridyl or substituted pyridyl; and
(c) if Ring A is thienyl, X¹ is N, R is substituted phenyl, R² is hydrogen, R^{B} is methyl, R¹ is —(CH₂)ₙ-L, n is 1, and L is —C(═O)O—R³, then R³ is not methyl or ethyl.

In certain embodiments, the bromodomain inhibitor of Formula (I) is of Formula (I-B):

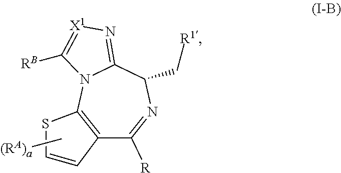

or pharmaceutically acceptable salt, solvate, hydrate, polymorph, co-crystal, tautomer, stereoisomer, isotopically labeled derivative, or prodrug thereof, wherein R^{1′} is hydrogen, —C(═O)O—R³, —C(═O)—R³, —C(═O)NR³R⁴, optionally substituted aryl, or optionally substituted aryl.

In certain embodiments, the bromodomain inhibitor of Formula (I) is of Formula (I-C):

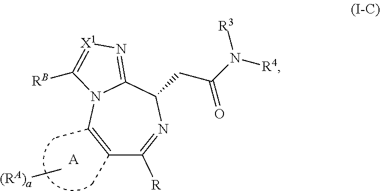

or pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (I) is of Formula (I-D):

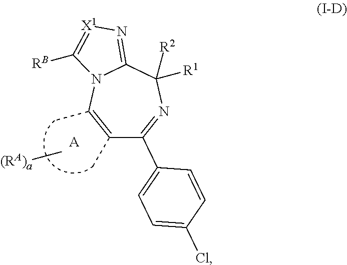

or pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (I) is of Formula (I-E):

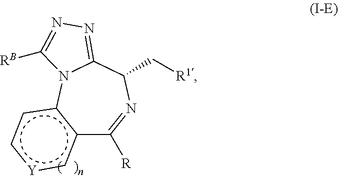

or pharmaceutically acceptable salt, solvate, hydrate, polymorph, co-crystal, tautomer, stereoisomer, isotopically labeled derivative, or prodrug thereof, wherein R^{1′} is hydrogen, —C(═O)O—R³, —C(═O)—R³, —C(═O)NR³R⁴, optionally substituted aryl, or optionally substituted aryl; Y is O, N, S, or CR^{A}; n is 0 or 1; and the dashed circle indicates an aromatic or non-aromatic ring.

In certain embodiments, the bromodomain inhibitor of Formula (I) is of Formula (I-F):

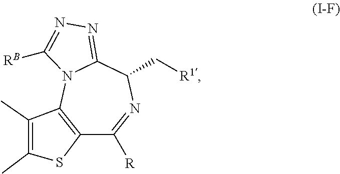

or pharmaceutically acceptable salt, solvate, hydrate, polymorph, co-crystal, tautomer, stereoisomer, isotopically labeled derivative, or prodrug thereof, wherein R^{1′} is hydrogen, —C(═O)O—R³, —C(═O)—R³, —C(═O)NR³R⁴, optionally substituted aryl, or optionally substituted aryl.
In certain embodiments, the bromodomain inhibitor of Formula (I) is of Formula (I-G):

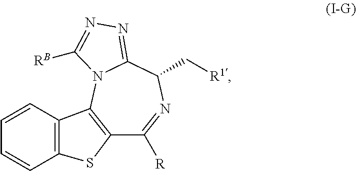

or pharmaceutically acceptable salt, solvate, hydrate, polymorph, co-crystal, tautomer, stereoisomer, isotopically labeled derivative, or prodrug thereof, wherein R^{1′} is hydrogen, —C(═O)O—R³, —C(═O)—R³, —C(═O)NR³R⁴, optionally substituted aryl, or optionally substituted aryl.

In certain embodiments, the bromodomain inhibitor of Formula (I) is of Formula (I-H):

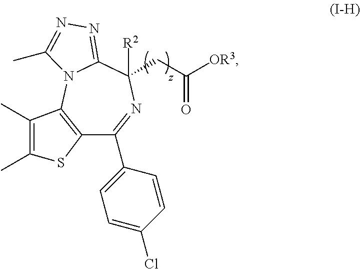

or pharmaceutically acceptable salt, solvate, hydrate, polymorph, co-crystal, tautomer, stereoisomer, isotopically labeled derivative, or prodrug thereof, wherein z is 1, 2, or 3, and R² is hydrogen, halogen, or unsubstituted C₁₋₆ alkyl.

In certain embodiments, the bromodomain inhibitor of Formula (I) is of Formula (I-J):

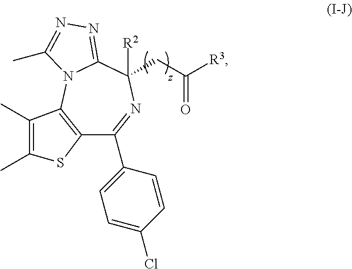

or pharmaceutically acceptable salt, solvate, hydrate, polymorph, co-crystal, tautomer, stereoisomer, isotopically labeled derivative, or prodrug thereof, wherein z is 1, 2, or 3, and R² is hydrogen, halogen, or unsubstituted C₁₋₆ alkyl.

In certain embodiments, the bromodomain inhibitor of Formula (I) of Formula (I-K):

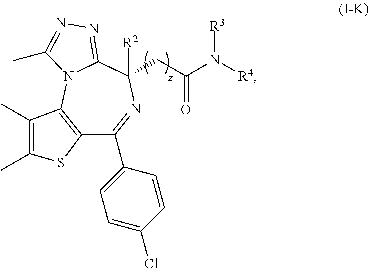

or pharmaceutically acceptable salt, solvate, hydrate, polymorph, co-crystal, tautomer, stereoisomer, isotopically labeled derivative, or prodrug thereof, wherein z is 1, 2, or 3, and R² is hydrogen, halogen, or unsubstituted C₁₋₆ alkyl.

In certain embodiments, the bromodomain inhibitor of Formula (I) is of Formula (I-L):

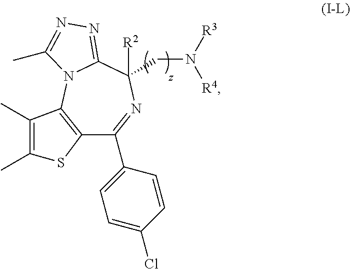

or pharmaceutically acceptable salt, solvate, hydrate, polymorph, co-crystal, tautomer, stereoisomer, isotopically labeled derivative, or prodrug thereof, wherein z is 1, 2, or 3, and R² is hydrogen, halogen, or unsubstituted C₁₋₆ alkyl.

In certain embodiments, the bromodomain inhibitor of Formula (I) is of Formula (I-M):

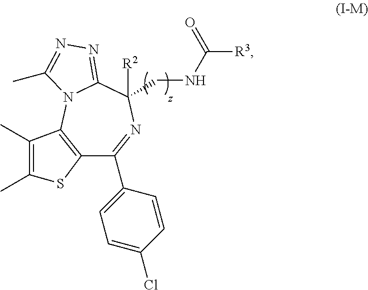

or pharmaceutically acceptable salt, solvate, hydrate, polymorph, co-crystal, tautomer, stereoisomer, isotopically labeled derivative, or prodrug thereof, wherein z is 1, or 3, wherein z is 1, 2, or 3, and R² is hydrogen, halogen, or unsubstituted C₁₋₆ alkyl.

In certain embodiments, the bromodomain inhibitor of Formula (I) is of Formula (I-N):

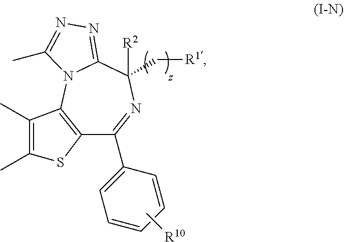

or pharmaceutically acceptable salt, solvate, hydrate, polymorph, co-crystal, tautomer, stereoisomer, isotopically labeled derivative, or prodrug thereof, wherein z is 1, 2, or 3; R² is hydrogen, halogen, or unsubstituted C₁₋₆ alkyl; R^{1′} is hydrogen, —C(═O)O—R³, —C(═O)—R³, —C(═O)NR³R⁴, optionally substituted aryl, or optionally substituted aryl; and R¹⁰ is hydrogen, halogen, optionally substituted alkyl, optionally substituted alkoxy, optionally substituted amino, or optionally substituted acyl, optionally substituted heterocyclyl, optionally substituted aryl, or optionally substituted heteroaryl.

In certain embodiments, the bromodomain inhibitor of Formula (I) is of Formula (I-O):

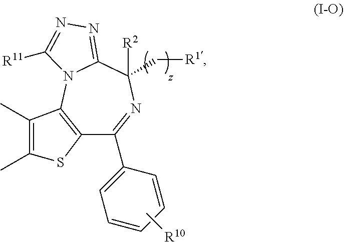

or pharmaceutically acceptable salt, solvate, hydrate, polymorph, co-crystal, tautomer, stereoisomer, isotopically labeled derivative, or prodrug thereof, wherein z is 1, 2, or 3; R² is hydrogen, halogen, or unsubstituted C₁₋₆ alkyl; R^{1′} is hydrogen, —C(═O)O—R³, —C(═O)—R³, —C(═O)NR³R⁴, optionally substituted aryl, or optionally substituted aryl; and R¹⁰ is hydrogen, halogen, optionally substituted alkyl, optionally substituted alkoxy, optionally substituted amino, or optionally substituted acyl, optionally substituted heterocyclyl, optionally substituted aryl, or optionally substituted heteroaryl; and R¹¹ is —OMe, —CH₂OH, —CH₂NH₂, or —CH₂OMe.

In certain embodiments, the bromodomain inhibitor of Formula (I) is of Formula (I-P):

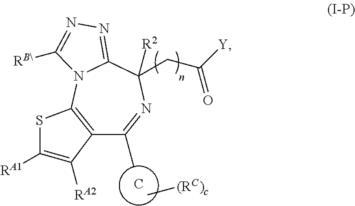

or a pharmaceutically acceptable salt, solvate, hydrate, polymorph, co-crystal, tautomer, stereoisomer, isotopically labeled derivative, or prodrug thereof, wherein.
Y is of formula:

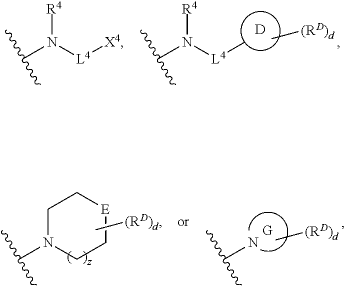

wherein:
   R⁴ hydrogen, optionally substituted alkyl, optionally substituted acyl, or a nitrogen protecting group;
   L¹ is optionally substituted alkylene;
   L⁴ is branched or substituted alkylene;
   X⁴ is halogen, —OR^{f}, —SR^{f}, or —N(R^{f})₂;
   Ring D is a carbocyclic or heterocyclic ring, wherein the heterocyclic ring contains exactly one heteroatom selected from N, O, or S;
   Ring G is a bicyclic heterocyclic or bicyclic heteroaryl ring, wherein the rings share exactly two atoms;
   E is —O—, —S—, —N(R^{E})—, or —CH(R^{E})—, wherein R^{E} is optionally substituted carbocyclyl optionally substituted heterocyclyl, optionally substituted aryl, or optionally substituted heteroaryl;
   each occurrence of R^{D} is independently hydrogen, halogen, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN, or two R^{D} attached to adjacent atoms are joined to form an optionally substituted carbocyclyl, optionally substituted heterocyclyl, optionally substituted aryl, optionally substituted heteroaryl ring;
   z is 0, 1, or 2; and
   d 0, 1, 2, 3, or 4;
R^{A1} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
R^{A2} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
X¹ is N or CR⁵, wherein R⁵ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN,
R^{B} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
Ring C is aryl or heteroaryl
each occurrence of R^{C} is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, optionally substituted sulfonyl, —OR^{f}, —SR^{f}, —N(R^{f})₂, —NO₂, or —CN;
c is 0, 1, 2, 3, or 4;
n is 0, 1, 2, 3, or 4;
R² is hydrogen, halogen, or optionally substituted alkyl; and
each occurrence of R^{f} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, optionally substituted sulfonyl, an oxygen protecting group, or a nitrogen protecting group, or two R^{f} are joined to form an optionally substituted heterocyclic or optionally substituted heteroaryl ring.

In certain embodiments, the compound of Formula (I) is a compound of Formula (I-P-i):

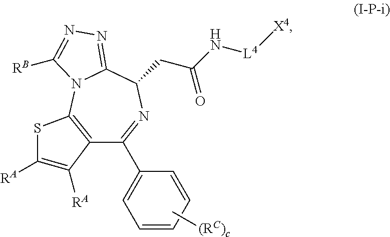

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-P-ii):

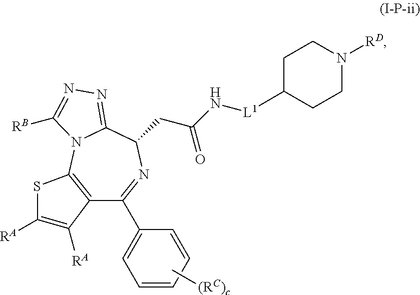

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-P-iii):

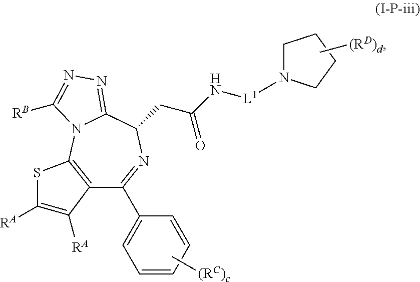

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-P-iv):

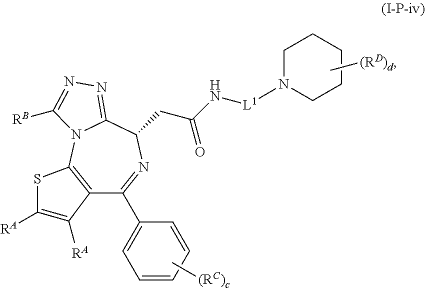

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-P-v):

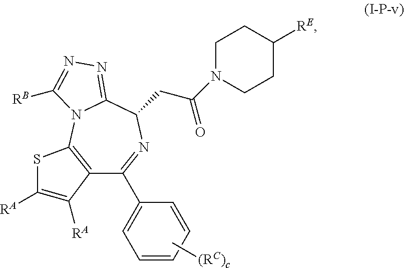

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-P-vi):

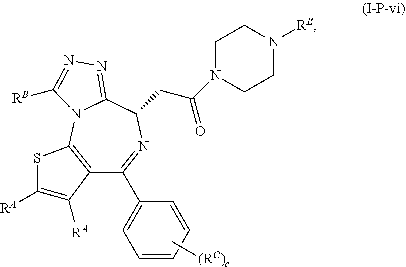

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-P-vii):

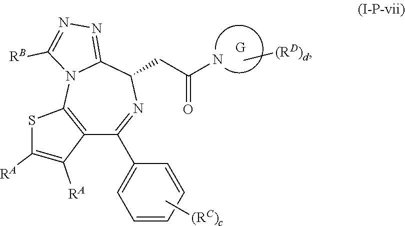

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-Q):

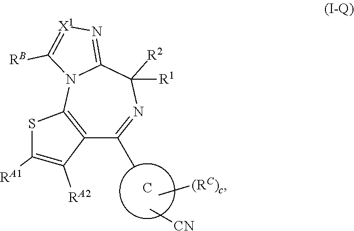

or a pharmaceutically acceptable salt, solvate, hydrate, polymorph, co-crystal, tautomer, stereoisomer, isotopically labeled derivative, or prodrug thereof, wherein:
R^{A1} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
R^{A2} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
X¹ is N or CR⁵, wherein R⁵ is hydrogen, halogen, optionally substituted alkyl optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
R^{B} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
Ring C is aryl or heteroaryl;
each occurrence of R^{C} is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, optionally substituted sulfonyl, —OR^{f}, —SR^{f}, —N(R^{f})₂, —NO₂, or —CN;
c is 0, 1, 2, 3, or 4;
R¹ is hydrogen, halogen, optionally substituted alkyl, or —(CH₂)ₙL, wherein n is 0, 1, 2, 3 or 4, and L is —C(═O)R³, —C(═O)OR³, —C(═O)NR³R⁴, —S(═O)₂R³, —S(═O)₂OR³, —S(═O)₂NR³R⁴, —OR³, —NR³R⁴, —N(R⁴)C(═O)R³, optionally substituted carbocyclyl, optionally substituted heterocyclyl, optionally substituted aryl, or optionally substituted heteroaryl;
R² is hydrogen, halogen, or optionally substituted alkyl;
each of R³ and R⁴ is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or optionally substituted acyl, an oxygen protecting group, or a nitrogen protecting group, or R³ and R⁴ are joined to form an optionally substituted heterocyclic or optionally substituted heteroaryl ring; and
each occurrence of R^{f} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, optionally substituted sulfonyl, an oxygen protecting group, or a nitrogen protecting group, or two R^{f} are joined to form an optionally substituted heterocyclic or optionally substituted heteroaryl ring.

In certain embodiments, the compound of Formula (I) is a compound of Formula (I-Q-i):

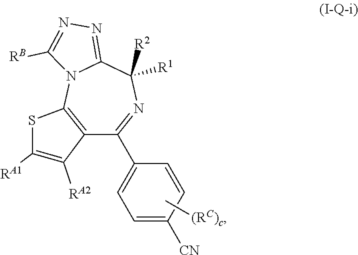

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-Q-ii):

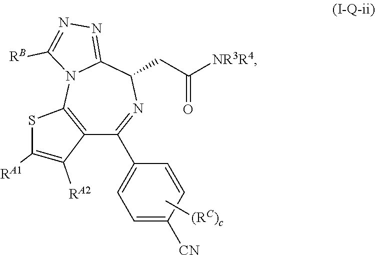

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-Q-iii):

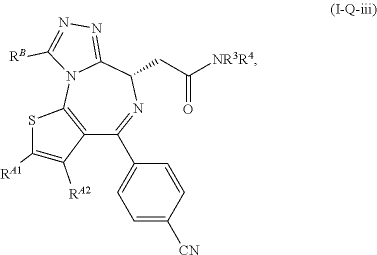

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-Q-iv):

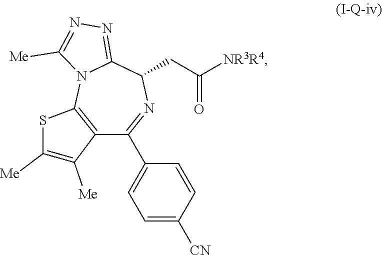

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-R):

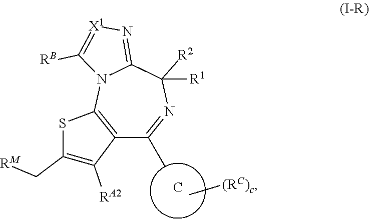

or a pharmaceutically acceptable salt, solvate, hydrate, polymorph, co-crystal, tautomer, stereoisomer, isotopically labeled derivative, or prodrug thereof, wherein.
R^{M} is —CN, N(R^{f})₂, or CH₂N(R^{f})₂;
R^{A2} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
X¹ is N or CR⁵, wherein R⁵ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
R^{B} is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{f}, —SR^{f}, —N(R^{f})₂, —NO₂, or —CN;
Ring C is aryl or heteroaryl;
each occurrence of R^{C} is independently halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, optionally substituted sulfonyl, —OR^{f}, —SR^{f}, —N(R^{f})₂, —NO₂, or —CN;
c is 0, 1, 2, 3, or 4;
R¹ is hydrogen, halogen, optionally substituted alkyl, or —(CH₂)ₙL, wherein n is 0, 1, 2, 3, or 4, and L is —C(═O)R³, —C(═O)OR³, —C(═O)NR³R⁴, —S(═O)₂OR³, —S(═O)₂NR³R⁴, —OR³, —NR³R⁴, —N(R⁴)C(═O)R³, optionally substituted carbocyclyl, optionally substituted heterocyclyl, optionally substituted aryl, or optionally substituted heteroaryl;
R² is hydrogen, halogen, or optionally substituted alkyl;
each R³ and R⁴ is independently hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl, or optionally substituted acyl, an oxygen protecting group, or a nitrogen protecting group, or R³ and R⁴ are joined to form an optionally substituted heterocyclic or optionally substituted heteroaryl ring; and
each occurrence of R^{f} is independently, hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, optionally substituted sulfonyl, an oxygen protecting group, or a nitrogen protecting group, or two R^{f} are joined to form an optionally substituted heterocyclic or optionally substituted heteroaryl ring.

In certain embodiments, the compound of Formula (I) is a compound of Formula (I-R-i):

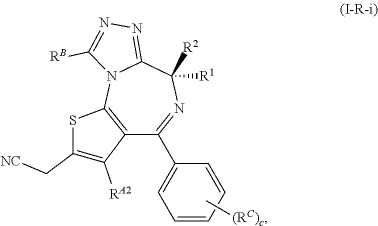

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-R-ii):

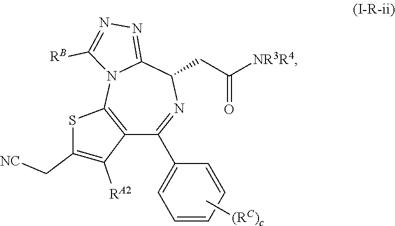

or a pharmaceutically acceptable salt, solvate, hydrate, polymorph, co-crystal, tautomer, stereoisomer, isotopically labeled derivative, or prodrug thereof.

In certain embodiments, the compound of Formula (I) is a compound of Formula (I-R-iii):

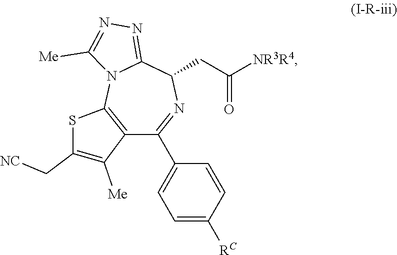

or a pharmaceutically acceptable salt, solvate, hydrate, polymorph, co-crystal, tautomer, stereoisomer, isotopically labeled derivative, or prodrug thereof.

In some embodiments, X is not N. In some embodiments, R is not substituted phenyl. In some embodiments, R^{B} is not methyl. In some embodiments, R³ is not methyl or ethyl.

In certain embodiments, the bromodomain inhibitor is of the formula:

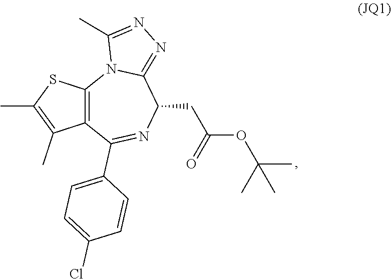 [SMILES: Cc1sc2c(c1C)C(c1ccc(Cl)cc1)=N[C@@H](CC(=O)OC(C)(C)C)c1nnc(C)n1-2]

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

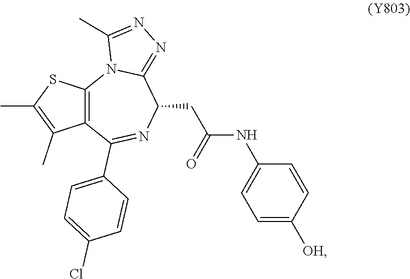 [SMILES: Cc1sc2c(c1C)C(c1ccc(Cl)cc1)=N[C@@H](CC(=O)Nc1ccc(O)cc1)c1nnc(C)n1-2]

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

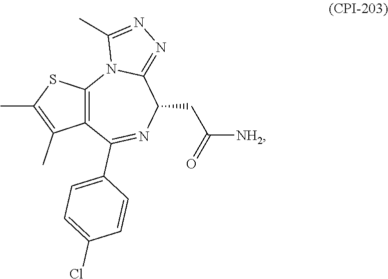 [SMILES: Cc1sc2c(c1C)C(c1ccc(Cl)cc1)=N[C@@H](CC(N)=O)c1nnc(C)n1-2]

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

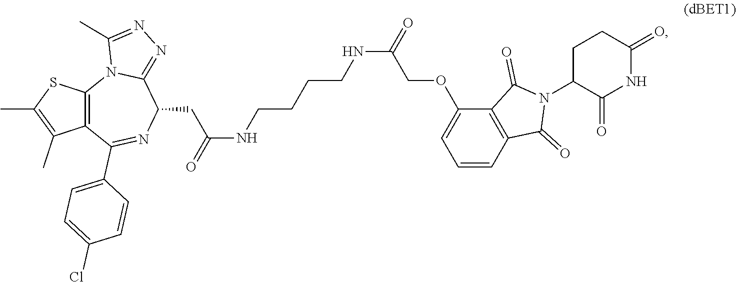 [SMILES: Cc1sc2c(c1C)C(c1ccc(Cl)cc1)=N[C@@H](CC(=O)NCCCCNC(=O)COc1cccc3c1C(=O)N(C1CCC(=O)NC1=O)C3=O)c1nnc(C)n1-2]

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

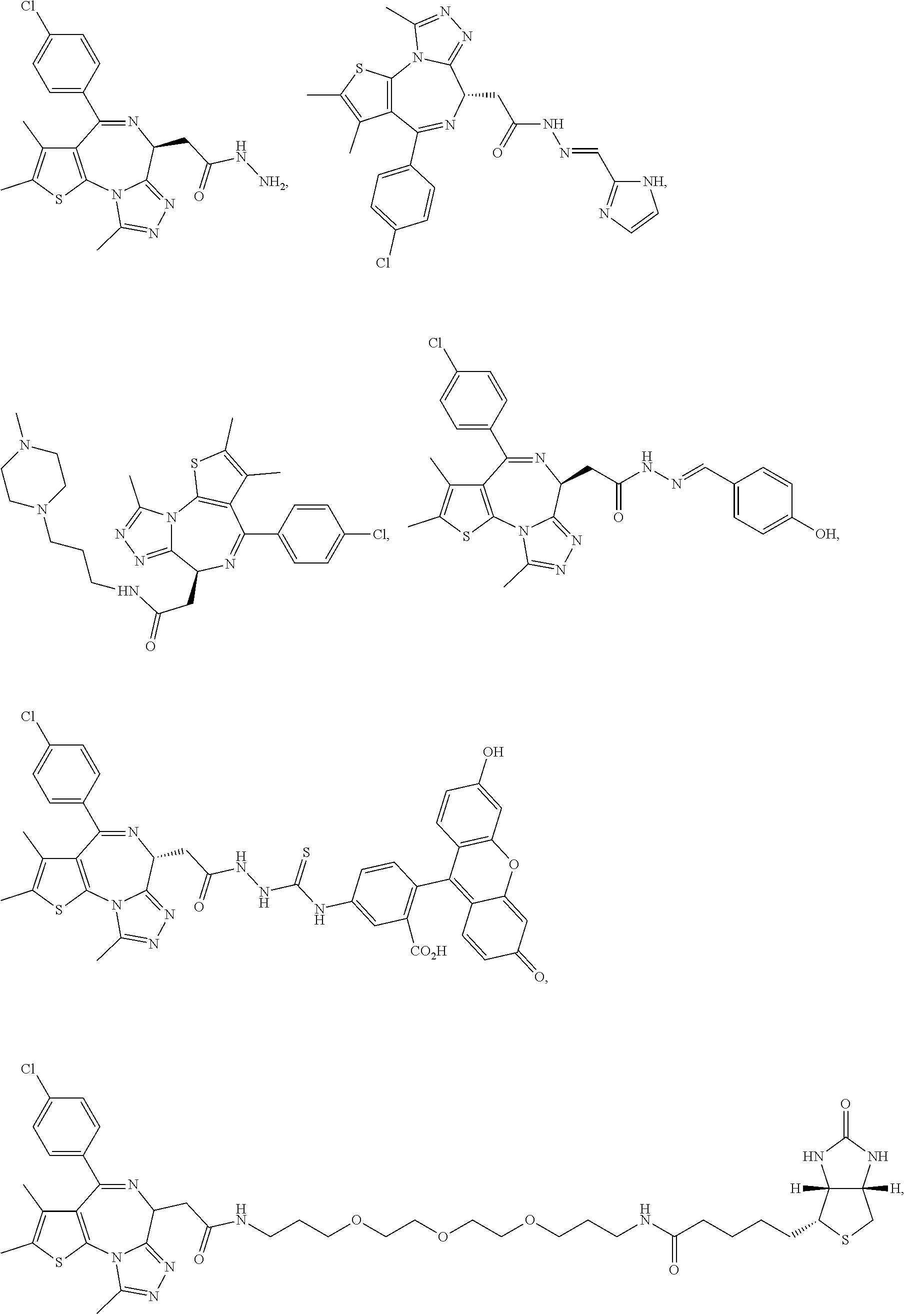

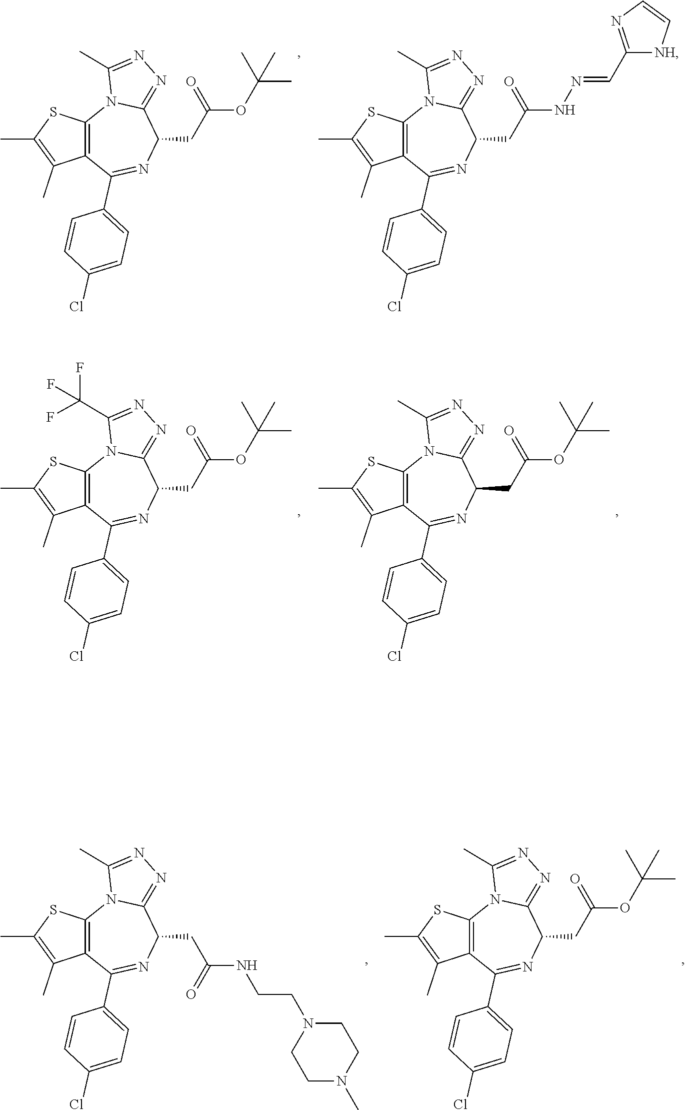

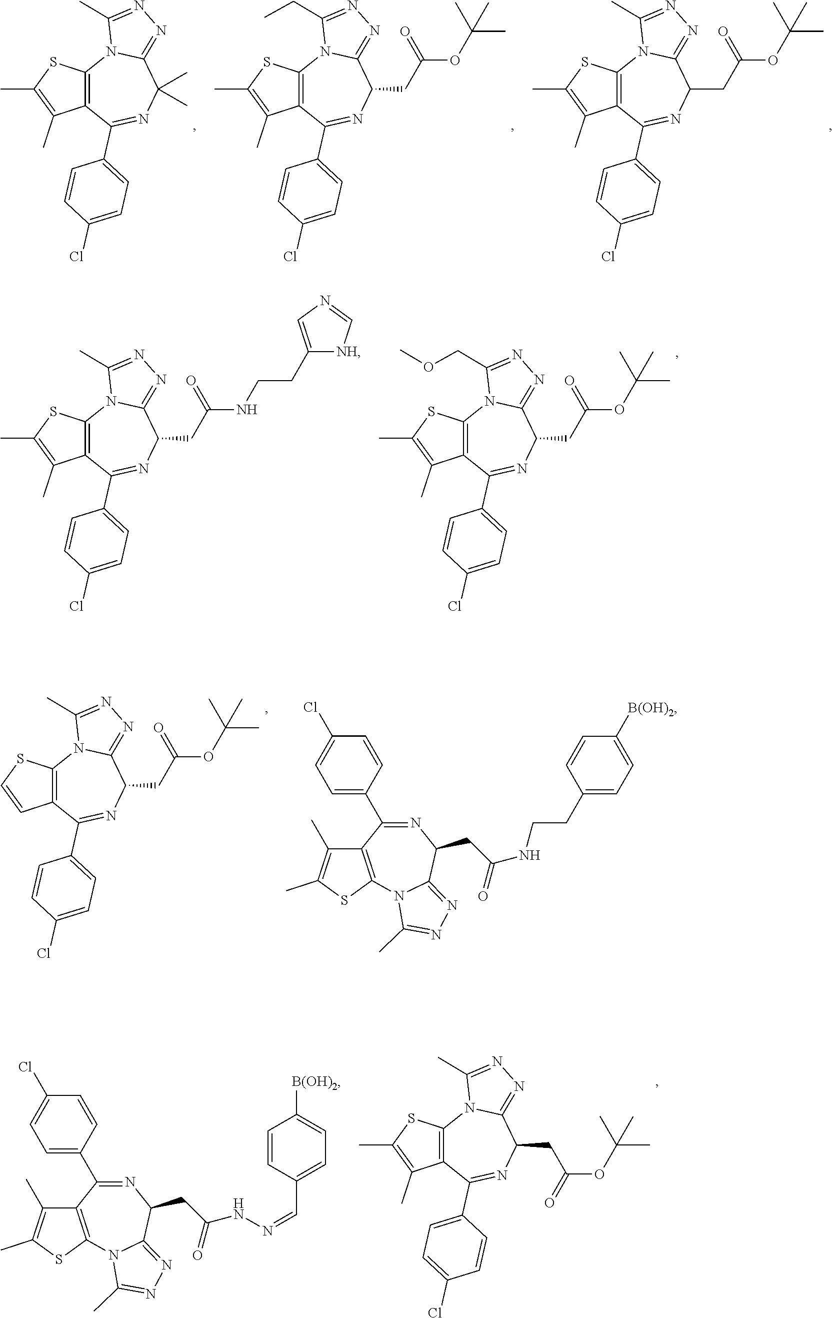

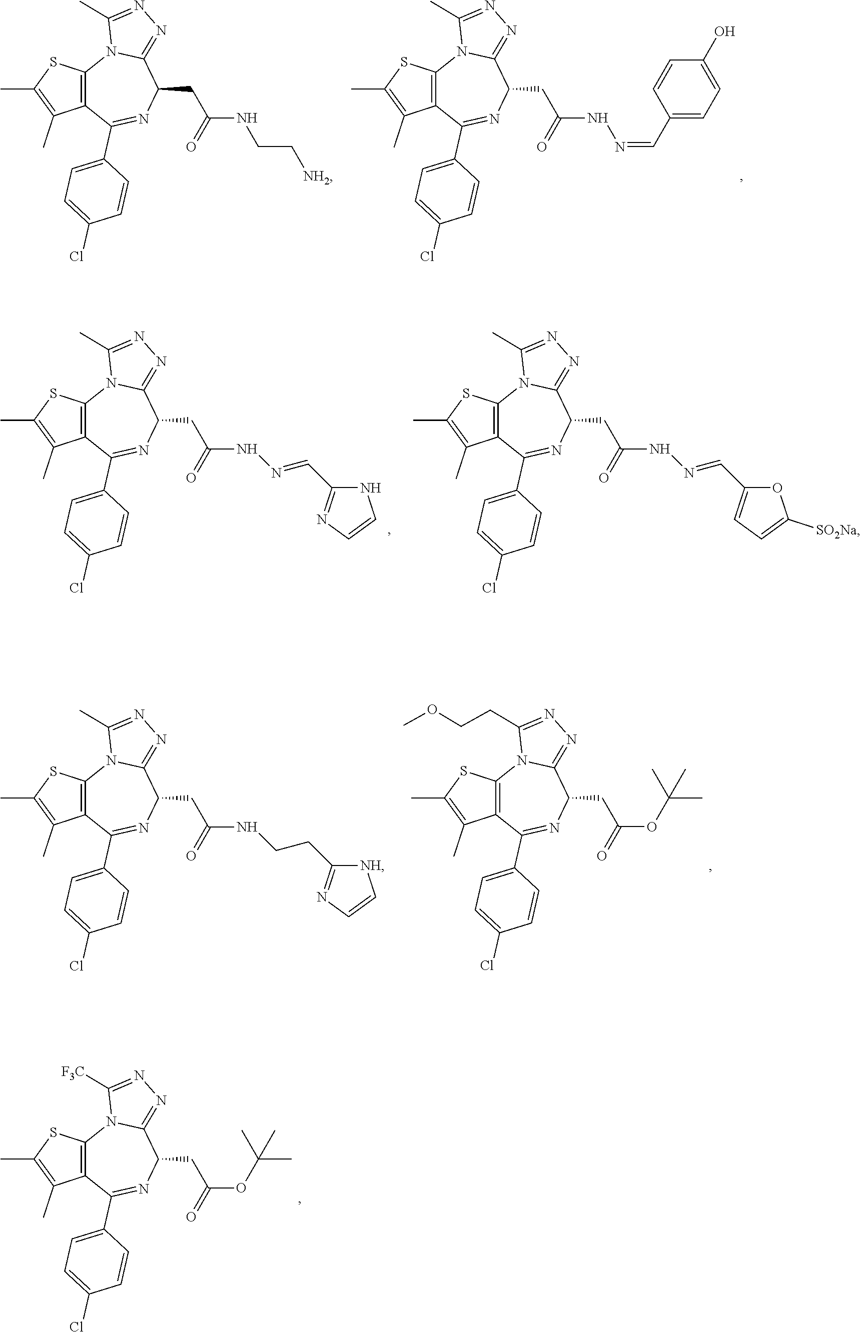

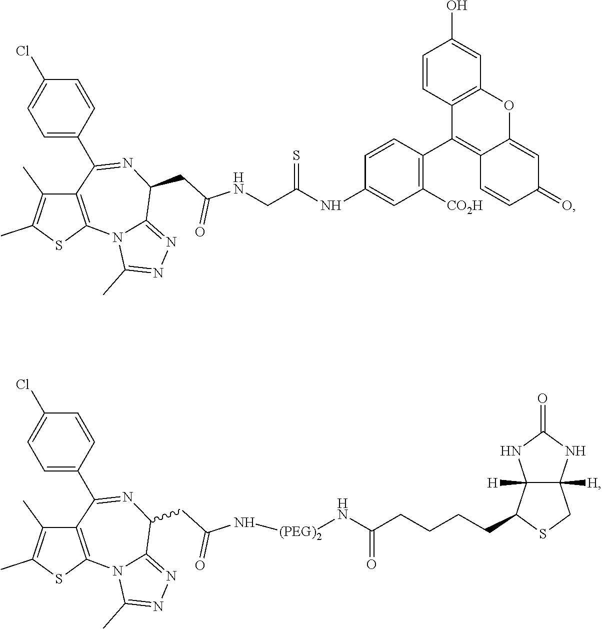

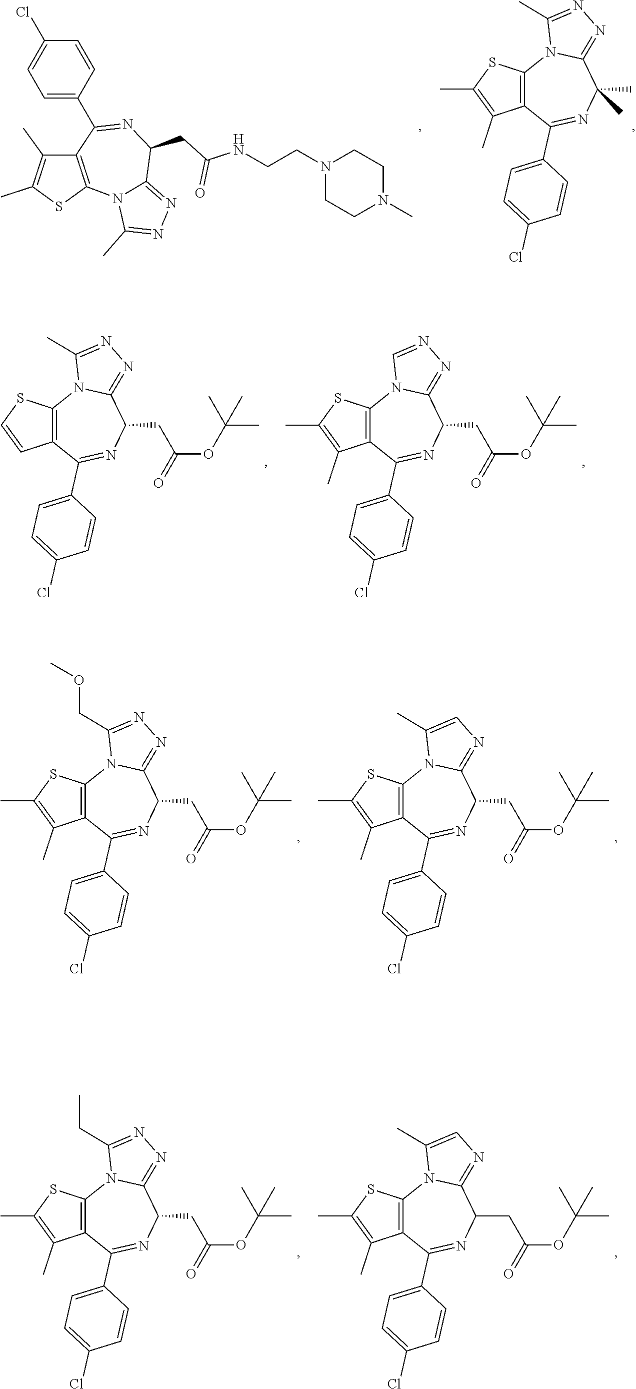

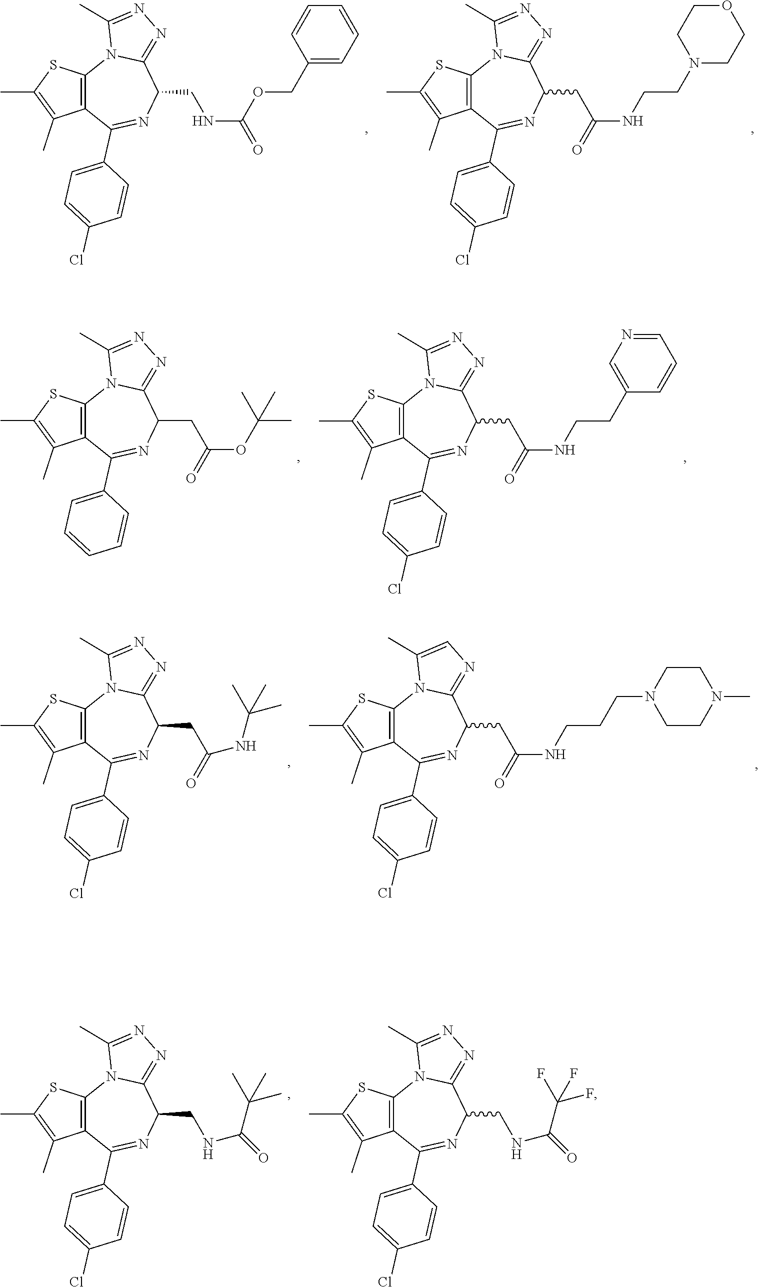

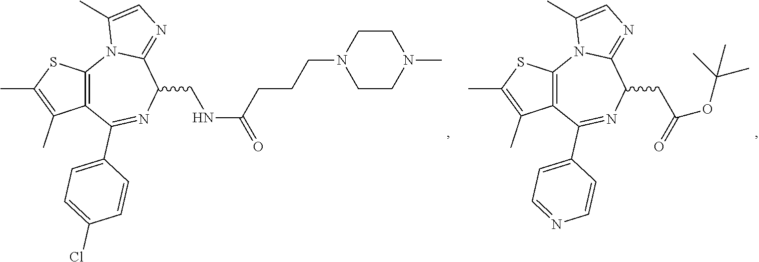

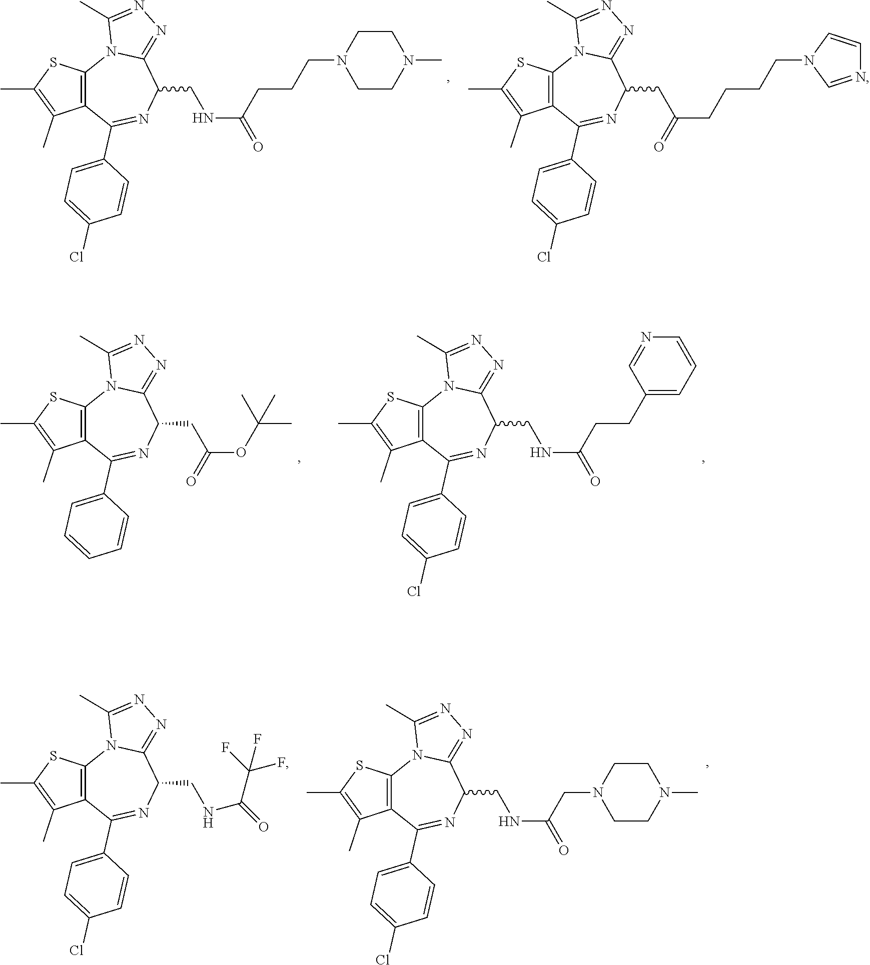

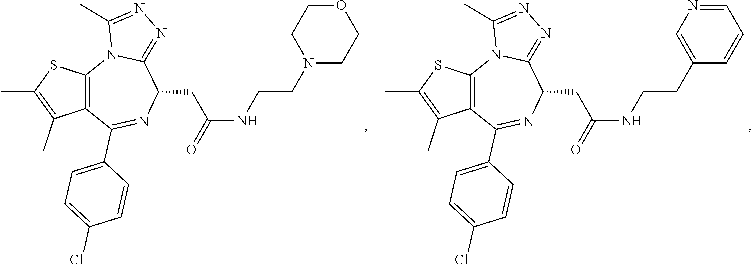 [SMILES: Cc1sc2c(c1C)C(c1ccc(Cl)cc1)=N[C@@H](CC(=O)NCCN1CCOCC1)c1nnc(C)n1-2 ; Cc1sc2c(c1C)C(c1ccc(Cl)cc1)=N[C@@H](CC(=O)NCCc1cccnc1)c1nnc(C)n1-2]

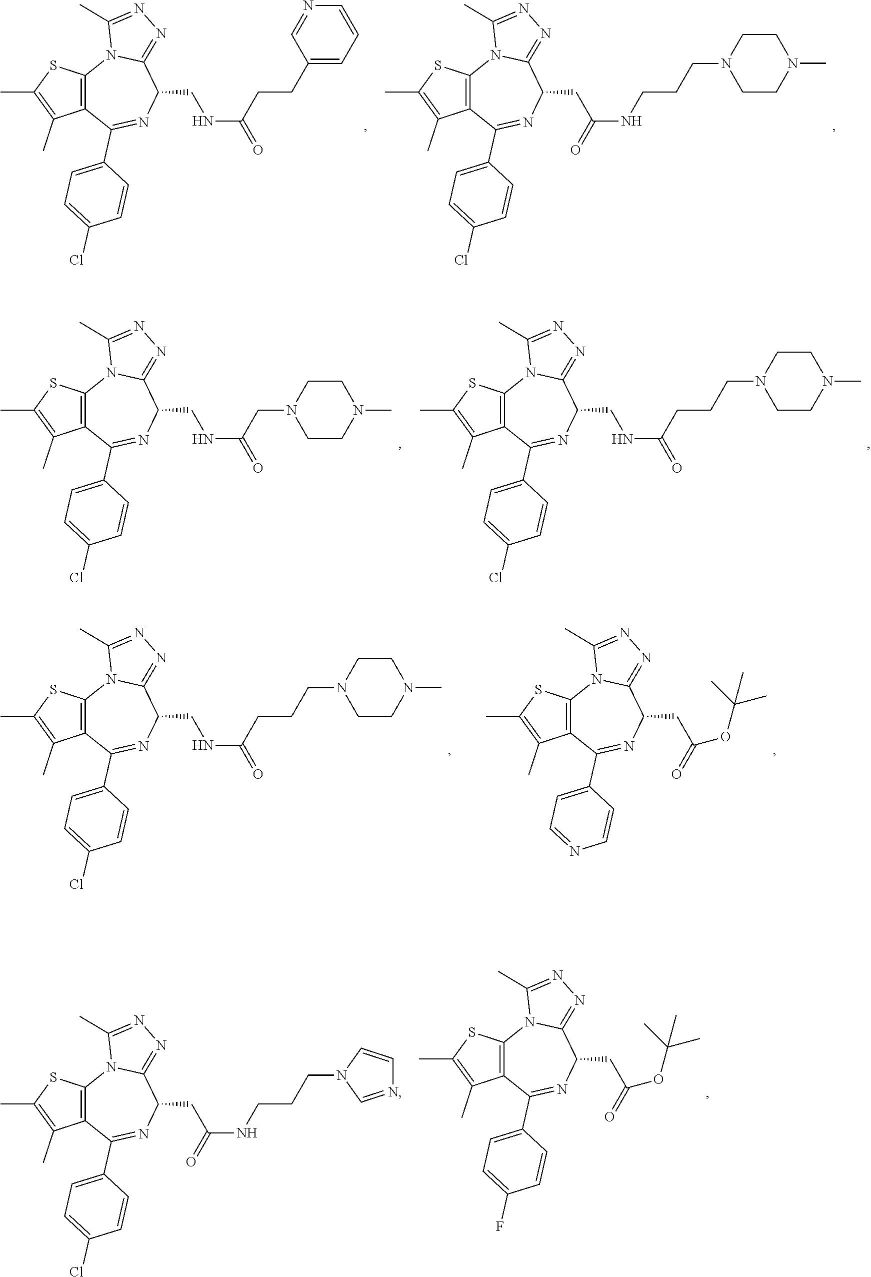

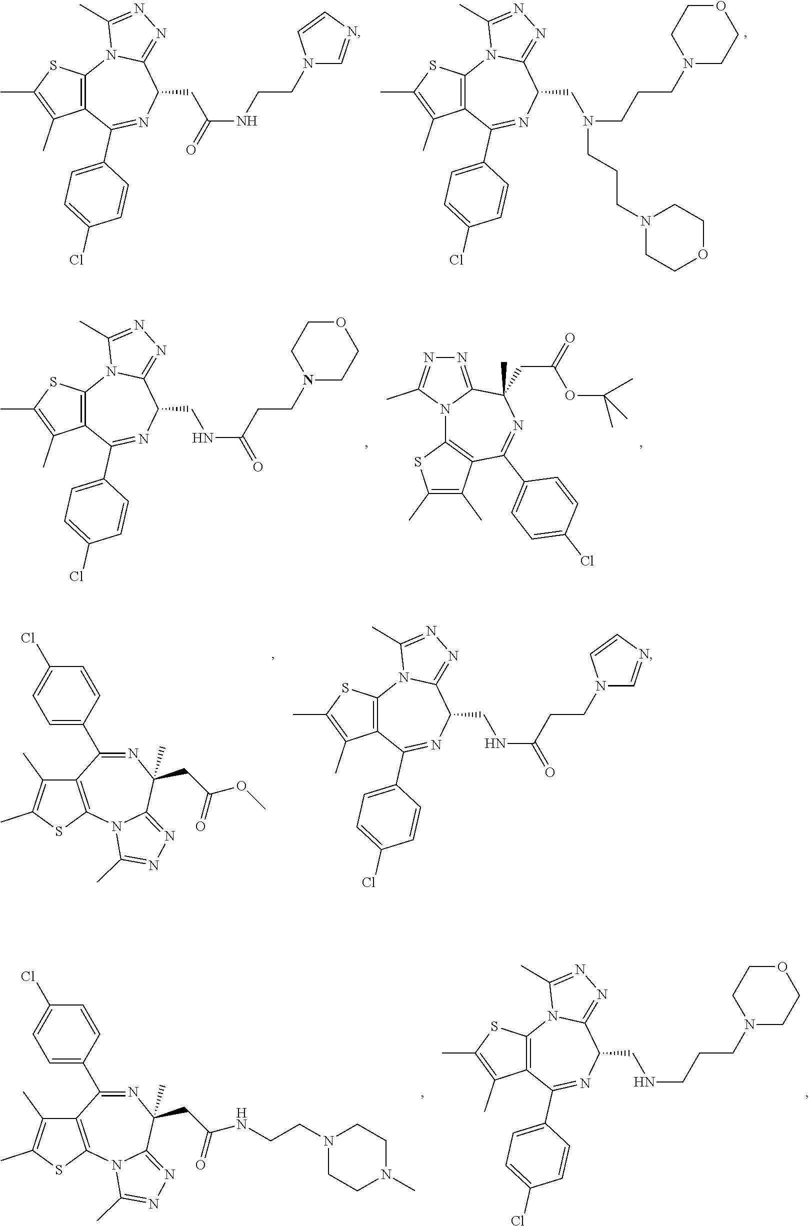

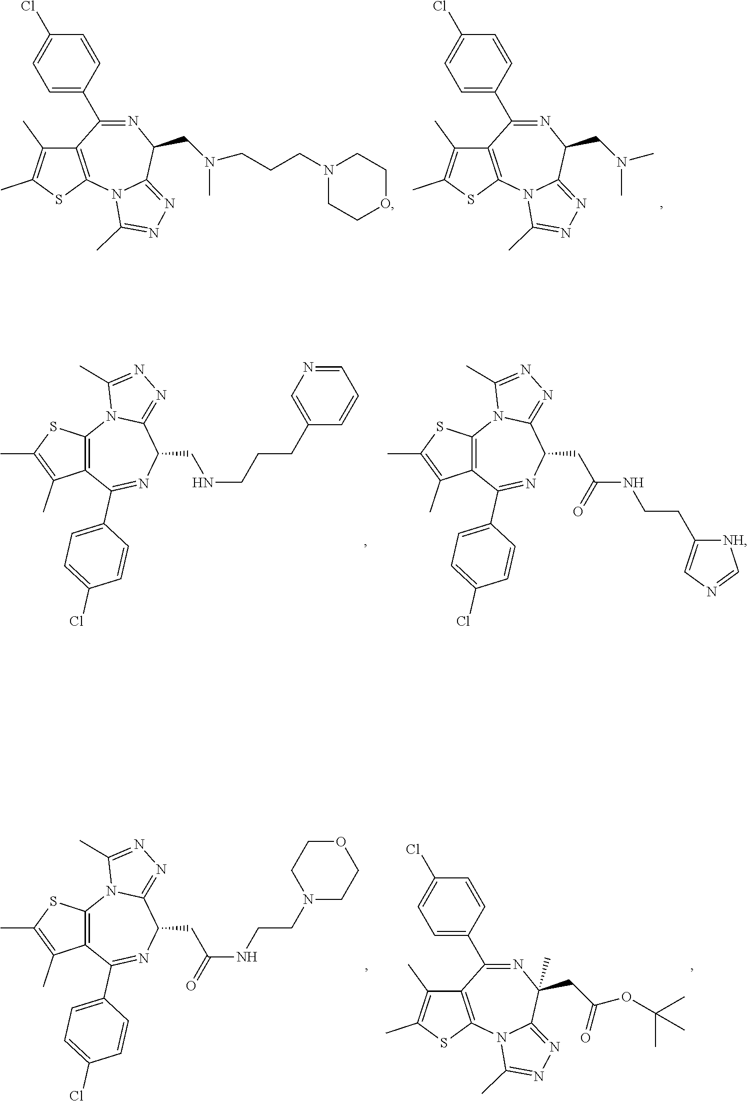

or a pharmaceutically acceptable salt, solvate, hydrate, polymorph, co-crystal, tautomer, stereoisomer, isotopically labeled derivative, or prodrug thereof.

### Compounds of Formula (II)

In certain embodiments, the bromodomain inhibitor is an inhibitor disclosed in international PCT Publication No. WO 2011/054846; international PCT Publication No. 2012/143416; U.S. Pat. No. 8,557,984; U.S. Pat. No. 8,846,709; U.S. Patent Publication No. US 2012/0232074; or U.S. Patent Publication No. US 2014/045834, each of which is incorporated herein by reference.

In certain embodiments, the bromodomain inhibitor is of Formula (II):

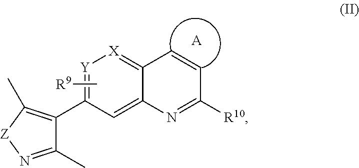

or a pharmaceutically acceptable salt, solvate, hydrate, polymorph, co-crystal, tautomer, stereoisomer, isotopically labeled derivative, or prodrug thereof, wherein:
A is of formula:

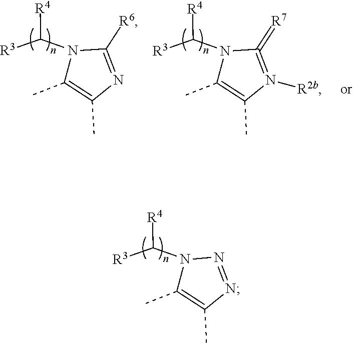

X is CH or N;
Y is CH or N;
Z is O or NH;
R³ is hydrogen, optionally substituted alkyl, optionally substituted carbocyclyl, optionally substituted heterocyclyl, optionally substituted aryl, or optionally substituted heteroaryl;
R⁴ hydrogen or optionally substituted alkyl;
R⁹ is hydrogen or optionally substituted alkoxy;
R¹⁰ is hydrogen, halogen, optionally substituted alkyl, or —CN;
R⁶ is hydrogen, optionally substituted alkyl, optionally substituted haloalkyl;
each of R^{a} and R^{b} independently is hydrogen, optionally substituted alkyl, or optionally substituted heterocyclyl, or R^{a} and R^{b} are joined to form an optionally substituted heterocyclyl ring;
R⁷ is ═O or ═S;
R^{2b} is hydrogen or optionally substituted alkyl; and
n is 0, 1, or 2.

In certain embodiments, the bromodomain inhibitor of Formula (II) is of Formula (II-A):

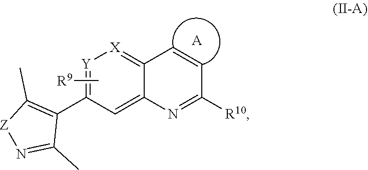

or a pharmaceutically acceptable salt, solvate, hydrate, polymorph, co-crystal, tautomer, stereoisomer, isotopically labeled derivative, or prodrug thereof, wherein:
A is of formula:

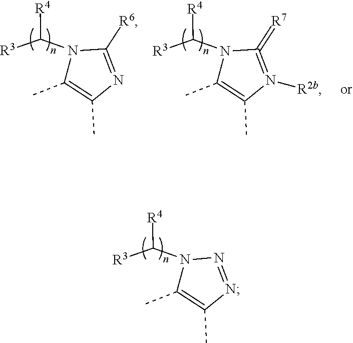

X is CH or N;
Y is CH or N;
Z is O or NH;
R³ is C₁₋₆ alkyl, C₃₋₆ carbocyclyl, 5- to 6-membered heterocyclyl, aryl, or heteroaryl, wherein each aryl or heteroaryl is optionally substituted by one to three groups selected from halogen, hydroxyl, —CN, —NO₂, C₁₋₆ alkyl, C₁₋₄ alkoxy, C₁₋₄ haloalkyl, C₁₋₄ haloalkoxy, —C(═O)(C₁₋₄ alkyl), —S(═O)₂(C₁₋₄ alkyl), —OS(═O)₂(C₁₋₄ alkyl), —NHS(═O)₂(C₁₋₄ alkyl), and C₁₋₄ alkyl substituted by hydroxy C₁₋₄ alkyoxy, or —S(═O)₂(C₁₋₄ alkyl);
R⁴ is hydrogen or C₁₋₆ alkyl;
R⁹ is hydrogen or C₁₋₆ alkoxy;
R¹⁰ is hydrogen, halogen, C₁₋₆ alkyl, or —CN;
R⁶ is hydrogen, C₁₋₆ alkyl, C₁₋₆ haloalkyl, —(CH₂)ₘCN, —(CH₂)OH, —(CH₂)(C₁₋₆ alkoxy), —(CH₂)(C₁₋₆ haloalkyl), —(CH₂)(C₁₋₆ haloalkoxy), —(CH₂)C(═O)NR^{a}R^{b}, —(CH₂)ₘOCH₃, —(CHR^{6a})ₚ(heteroaryl), —(CHR^{6a})ₚ(heterocyclyl), or —(CHR^{6a})ₚ(phenyl) substituted by C₁₋₆ alkyl, C₁₋₆ alkoxy, C₁₋₄ haloakl, C₁₋₄ haloalkoxy, or —CN;
each of R^{a} and R^{b} independently is hydrogen, C₁₋₆ alkyl, or heterocyclyl, or R^{a} and R^{b} are joined to form a 5- to 6-membered heterocyclyl ring;
R^{6a} is hydrogen or C₁₋₆ alkyl;
R⁷ is ═O or ═S;
R^{2b} is hydrogen, C₁₋₆ alkyl, —(CH₂)(C₁₋₆ alkoxy), —(CH₂)ₘCN, —(CH₂)OH, —(CH₂)ₘ(phenyl), or —(CHR²)ₘ(heterocyclyl);
m is 1, 2, or 3;
p is 0, 1, or 2; and
n is 0, 1, or 2.

In certain embodiments, the bromodomain inhibitor is of Formula (II-B):

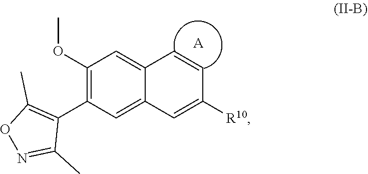

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of Formula (II-C):

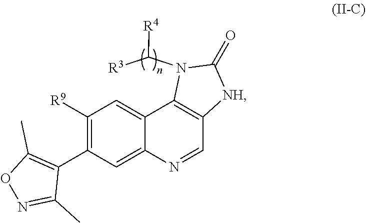

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of Formula (II-D):

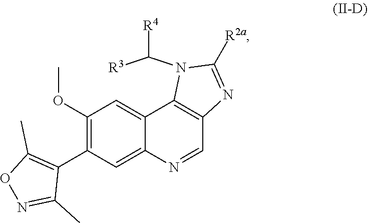

or a pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (II) is of Formula (II-E):

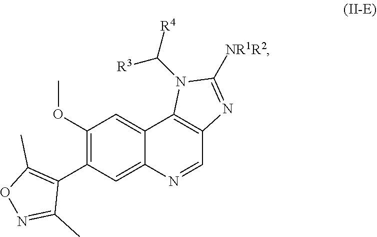

or a pharmaceutically acceptable salt, solvate, hydrate, polymorph, co-crystal, tautomer, stereoisomer, isotopically labeled derivative, or prodrug thereof, wherein:
R¹ is hydrogen or optionally substituted alkyl;
R² is hydrogen or optionally substituted alkyl;
or R¹ and R² are joined to form an optionally substituted heterocyclyl ring;
R³ is optionally substituted alkyl optionally substituted aryl optionally substituted heteroaryl, optionally substituted heterocyclyl, or optionally substituted carbocyclyl; and
R⁴ is hydrogen or optionally substituted alkyl.

In certain embodiments, the bromodomain inhibitor is of Formula (II-F):

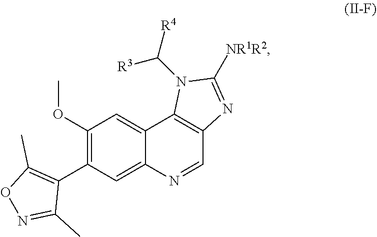

or a pharmaceutically acceptable salt, solvate, hydrate, polymorph, co-crystal, tautomer, stereoisomer, isotopically labeled derivative, or prodrug thereof, wherein:
R¹ is hydrogen or C₁₋₃ alkyl;
R² is hydrogen, C₁₋₆ alkyl, or C₂₋₆ alkyl substituted by one or more groups selected from hydroxy, C₁₋₄ alkoxy, and —NR^{a}R^{b}, wherein each of R^{a} and R^{b} is independently hydrogen or C₁₋₄ alkyl, or R^{a} and R^{b} are joined to form a heterocyclyl ring;
or R¹ and R² are joined to form a heterocyclyl ring;
R³ is hydrogen, C₁₋₃ alkyl, or —CH₂OH; and
R⁴ is phenyl optionally substituted with one or more groups selected from C₁₋₄ alkyl, —CF₃, halogen, hydroxy, and C₁₋₄ alkoxy, tetrahydropyranyl, tetrahydrofuranyl, C₃₋₇ carbocyclyl, —CH₂OMe, and heteroaryl optionally substituted with one or more C₁₋₄ alkyl, —CF₃, halogen, hydroxy, or C₁₋₄ alkoxy.

In certain embodiments, the bromodomain inhibitor is of formula:

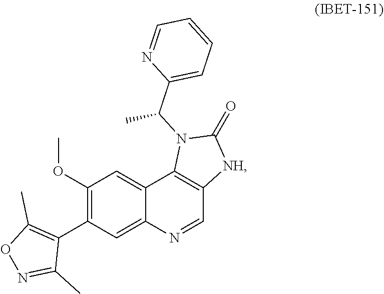 [SMILES: COc1cc2c(cc1-c1c(C)noc1C)ncc1[nH]c(=O)n([C@H](C)c3ccccn3)c12]

or a pharmaceutically acceptable salt, solvate, hydrate, polymorph, co-crystal, tautomer, stereoisomer, isotopically labeled derivative, or prodrug thereof.

### Compounds of Formula (III)

In certain embodiments, the bromodomain inhibitor is an inhibitor disclosed in international PCT Publication No. WO 2011/054845 or U.S. Patent Publication No. US 2012/0252781, each of which is incorporated herein by reference.

In certain embodiments, the bromodomain inhibitor is of Formula (III):

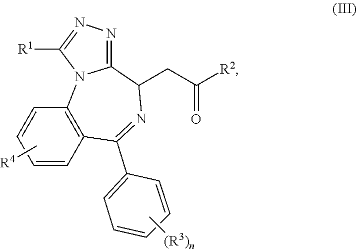

or a pharmaceutically acceptable salt, solvate, hydrate, polymorph, co-crystal, tautomer, stereoisomer, isotopically labeled derivative, or prodrug thereof, wherein:
R¹ is optionally substituted alkyl;
R² is —NR^{2a}R^{2a′} or —OR^{2b};
each of R^{2a}, R^{2a′}, and R^{2b} is independently optionally substituted alkyl, optionally substituted haloalkyl, or optionally substituted carbocyclyl, wherein any two adjacent groups on a carbocyclylic ring may be joined to form an optionally substituted carbocyclyl, optionally substituted heterocyclyl, optionally substituted aryl, or optionally substituted heteroaryl ring;
or R^{2a} and R^{2′} are joined to form an optionally substituted carbocyclyl or optionally substituted heterocyclyl ring;
each of R^{2c} and R^{2c′} is independently hydrogen or optionally substituted alkyl;
each instance of R³ is independently hydrogen, hydroxyl, halogen, optionally substituted alkyl, optionally substituted haloalkyl, optionally substituted alkoxy, optionally substituted haloalkoxy, —NO₂, —CN, or —C(═O)OR⁵;
R⁴ is hydroxyl, halogen, optionally substituted alkyl, optionally substituted haloalkyl, optionally substituted alkyl, optionally substituted haloalkyl, optionally substituted alkoxy, optionally substituted haloalkoxy, —NO₂, —CN, —C(═O)OR⁵, or —OS(═O)₂(alkyl);
R⁵ is optionally substituted alkyl; and
n is 1, 2, 3, 4, or 5.

In certain embodiments, the bromodomain inhibitor of Formula (III) is of Formula (III-A):

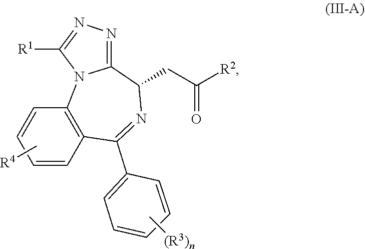

or a pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (III) is of Formula (III-B):

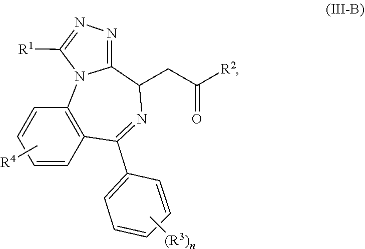

or a pharmaceutically acceptable salt, solvate, hydrate, polymorph, co-crystal, tautomer, stereoisomer, isotopically labeled derivative, or prodrug thereof, wherein:
R¹ is C₁₋₃ alkyl;
R² is —NR^{2a}R^{2a′} or —OR^{2b};
each of R^{2a}, R^{2a′}, and R^{2b} is independently C₁₋₆ alkyl, C₁₋₆ haloalkyl, carbocyclyl, C₁₋₆ alkyl substituted by —NR^{2c}R^{2c′}, or C₁₋₄ alkyl substituted by carbocyclyl or heterocyclyl, wherein each instance of carbocyclyl or heterocyclyl is optionally substituted by one or more groups selected from halogen, C₁₋₆ alkyl, C₁₋₆ haloalkyl, C₁₋₆ alkyoxy, C₁₋₆ haloalkoxy, carbonyl, —C(═O)(carbocyclyl), amino, hydroxyl, —N₃, —NO₂, and —CN, wherein —C(—O)(carbocyclyl) is optionally substituted by one or more groups selected from halogen, C₁₋₆ alkyl, C₁₋₆ haloalkyl, C₁₋₆ alkyoxy, C₁₋₆ haloalkoxy, —N₃, —NO₂, and —CN; or two adjacent groups on any of the carbocyclyl or heterocyclyl groups may be joined to form a 5- or 6-membered carbocyclyl, heterocyclyl, acyl, or heteroaryl ring containing up to 2 heteroatoms independently selected from O, S, and N;
or R^{2a} and R^{2a′} are joined to form a 4- to 7-membered carbocyclyl or heterocyclyl ring containing up to 2 heteroatoms independently selected from O, S, or N, wherein the ring is optionally substituted by one or more groups selected from C₁₋₆ alkyl hydroxyl, or amino;
provided that when R² and R^{2a′} are not joined to form a ring, one of R^{2a} and R^{2a′} is hydrogen;
each of R^{2c} and R^{2a′} is independently hydrogen or C₁₋₆ alkyl;
each instance of R³ is independently hydrogen, hydroxyl, halogen, C₁₋₆ alkyl, C₁₋₆ haloalkyl, C₁₋₆ alkoxy, C₁₋₆ haloalkoxy, —NO₂, —CN, —CF₃, —OCF₃, —C(═O)OR⁵, or C₁₋₄ alkyl substituted by —N^{2c}R^{2c′} or —OH;
R⁴ is hydroxyl, halogen, C₁₋₆ alkyl, C₁₋₆ haloalkyl, C₁₋₆ alkoxy, C₁₋₆ haloalkoxy, —NO₂, —CN, —CF₃, —OCF₃, —C(═O)OR⁵, or —OS(═O)₂(C₁₋₄ alkyl);
R⁵ is C₁₋₃ alkyl; and
n is 1, 2, 3, 4, or 5.

In certain embodiments, the bromodomain inhibitor of Formula (III) is of Formula (III-C):

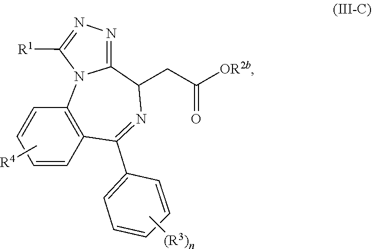

or a pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (III) is of Formula (III-D):

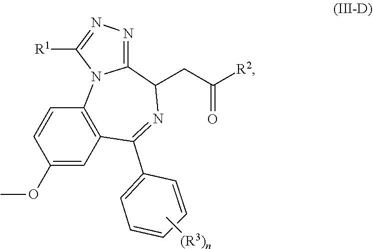

or a pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (III) is of Formula (III-E):

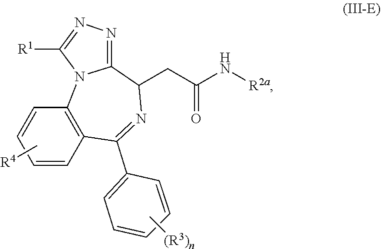

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of formula:

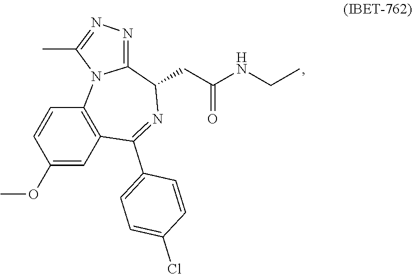 [SMILES: CCNC(=O)C[C@@H]1N=C(c2ccc(Cl)cc2)c2cc(OC)ccc2-n2c(C)nnc21]

or a pharmaceutically acceptable salt, solvate, hydrate, polymorph, co-crystal, tautomer, stereoisomer, isotopically labeled derivative, or prodrug thereof.

### Compounds of Formula (IV)

In certain embodiments, the bromodomain inhibitor is an inhibitor disclosed in international PCT Publication No. WO 2008/092231; U.S. Pat. No. 8,053,440; U.S. Pat. No. 8,889,698; U.S. Patent Publication No. 2008/0188467; U.S. Patent Publication No. US 2012/015905, or U.S. Patent Publication No. US 2015/0072955, each of which is incorporated herein by reference.

In certain embodiments, the bromodomain inhibitor is of Formula (IV):

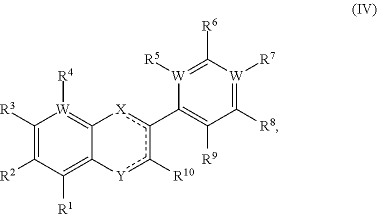

or a pharmaceutically acceptable salt, solvate, hydrate, polymorph, co-crystal, tautomer, stereoisomer, isotopically labeled derivative, or prodrug thereof, wherein:
X is CR¹¹, N, or N^{R11};
Y is —C(═O)—, —C(═S)—, —S(═O)₂—;
R¹¹ is hydrogen halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;
each of R¹ and R³ is independently hydrogen, halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;

R² is hydrogen, halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;
each of R⁶ and R⁸ is independently hydrogen, halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;
each of R⁴ and R⁵ is independently absent, hydrogen, halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;
R⁹ is hydrogen, halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;
R⁷ is absent, hydrogen, halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;
R¹⁰ is hydrogen, halogen, optionally substituted alkyl, optionally substituted heteroalkyl, optionally substituted alkenyl, optionally substituted alkynyl, optionally substituted alkoxy, optionally substituted amido, optionally substituted amino, or hydroxyl;
or two substituents attached to adjacent atoms and selected from R¹, R², R³, R⁶, R⁷, R⁸, and R¹⁰, are joined to form an optionally substituted carbocyclyl, optionally substituted heterocyclyl, optionally substituted aryl, or optionally substituted heteroaryl ring;
each W is independently C or N, wherein if W is N the attached substituent R⁴, R⁵, or R⁷ is absent; and
each [symbol] is independently a single or double bond provided two adjacent [symbol] are not both double bonds.

In certain embodiments, the bromodomain inhibitor of Formula (IV) is of Formula (IV-A):

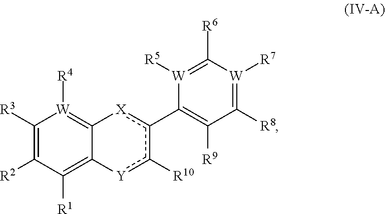

or a pharmaceutically acceptable salt, solvate, hydrate, polymorph, co-crystal, tautomer, stereoisomer, isotopically labeled derivative, or prodrug thereof, wherein:
X is CR¹¹, N, or N^{R11};
Y is —C(═O)—, —C(═S)—, —S(═O)₂—;
R¹¹ is hydrogen, unsubstituted alkyl, unsubstituted alkenyl, or unsubstituted alkynyl;
each of R¹ and R³ is independently hydrogen, halogen, alkyl, alkoxy, or amino;
R² is hydrogen, halogen, alkyl, alkenyl, alkoxy, amido, or amino;
each of R⁶ and R⁸ is independently hydrogen, halogen, alkyl, alkoxy, or amino;
each of R⁴ and R⁵ is independently absent, hydrogen, or halogen;
R⁹ is hydrogen or halogen;
R⁷ is absent, hydrogen, alkyl, alkenyl, alkoxy, amido, amino, hydroxyl, or heteroalkyl wherein the heteroatom is oxygen;
R¹⁰ is hydrogen or alkyl;
or two substituents attached to adjacent atoms and selected from R¹, R², R³, R⁶, R⁷, R⁸, and R¹⁰, are joined to form a carbocyclyl, heterocyclyl, aryl, or heteroaryl ring;
each W is independently C or N, wherein if W is N the attached substituent R⁴, R⁵, or R⁷ is absent; and
each [symbol] is independently a single or double bond, provided two adjacent [symbol] are not both double bonds.

In certain embodiments, the bromodomain inhibitor of Formula (IV) is of Formula (IV-B):

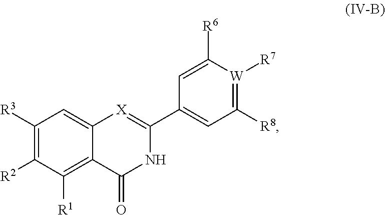

or a pharmaceutically acceptable salt, solvate, hydrate, polymorph, co-crystal, tautomer, stereoisomer, isotopically labeled derivative, or prodrug thereof, wherein:
X is N or CH;
each of R¹ and R³ is independently hydrogen or alkoxy;
R² is hydrogen, halogen, alkyl, or alkoxy;
each of R⁶ and R⁸ is independently hydrogen, chloride, alkyl, alkoxy; and
R⁷ is absent, alkoxy, amino, hydroxyl, or alkyl substituted with heterocyclyl.

In certain embodiments, the bromodomain inhibitor is of formula:

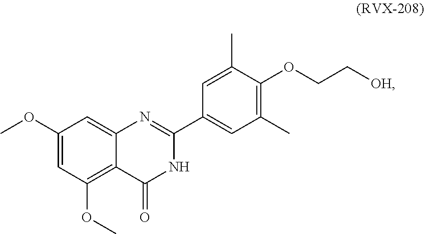 [SMILES: COc1cc(OC)c2c(=O)[nH]c(-c3cc(C)c(OCCO)c(C)c3)nc2c1]

or a pharmaceutically acceptable salt, solvate, hydrate, polymorph, co-crystal, tautomer, stereoisomer, isotopically labeled derivative, or prodrug thereof.

### Compounds of Formula (V)

In certain embodiments, the bromodomain inhibitor is an inhibitor disclosed in international PCT Publication No. WO 2015/013635, which is incorporated herein by reference.

In certain embodiments, the bromodomain inhibitor is of Formula (V):

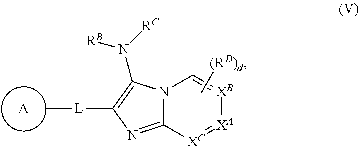

or a pharmaceutically acceptable salt, solvate, hydrate, polymorph, co-crystal, tautomer, stereoisomer, isotopically labeled derivative, or prodrug thereof, wherein:
X^{A} is C(R^{D}) or N;
X^{B} is C(R^{D}) or N;
X^{C} is C(R^{D}) or N;
wherein no more than two of X^{A}, X^{b}, and X^{C} can be N;
Ring A is of the formula:

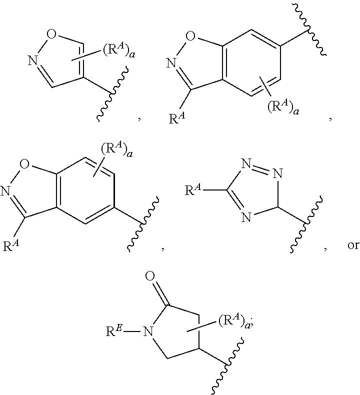

L is a bond or of the formula:

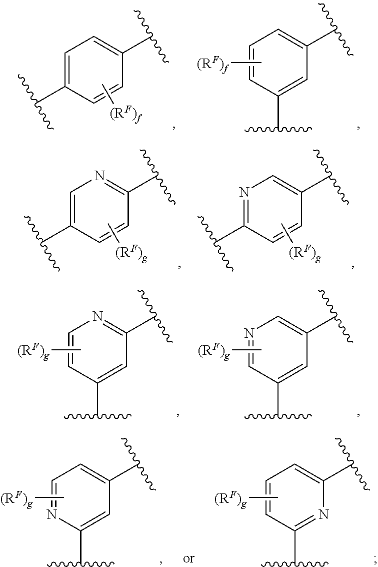

each instance of R^{A} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A1}, —N(R^{A1})₂, —SR^{A1}, —CN, —SCN, —C(═NR^{A1})R^{A1}, —C(═NR^{A1})OR^{A1}, —C(═NR^{A1})N(R^{A1})₂, —C(═O)R^{A1}, —C(═O)OR^{A1}, —C(═O)N(R^{A1})₂, —NO₂, —NR^{A1}C(═O)R^{A1}, —NR^{A1}C(═O)OR^{A1}, —NR^{A1}C(═O)N(R^{A1})₂, —OC(═O)R^{A1}, —OC(═O)OR^{A1}, or —OC(═O)N(R^{A1})₂, or about two instances of R^{A} are joined to form a substituted or unsubstituted carbocyclic, substituted or unsubstituted heterocyclic, substituted or unsubstituted aryl, or substituted or unsubstituted heteroaryl ring;
each instance of R^{A1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two instances of R^{A1} are joined to form a substituted or unsubstituted heterocyclic ring;
R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl substituted or unsubstituted heterocyclyl, substituted or unsubstituted aryl, substituted or unsubstituted heteroaryl, —C(═O)R^{B1}, —C(═O)OR^{B1}, —C(═O)N(R^{B1})₂, or a nitrogen protecting group, or R^{B} and R^{C} are joined to form a substituted or unsubstituted heterocyclic ring;
each instance of R^{B1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or an oxygen protecting group when attached to an oxygen atom, or about two instances of R^{B1} are joined to form a substituted or unsubstituted heterocyclic ring;
R^{C} is hydrogen, substituted or unsubstituted acyl, substituted or unsubstituted substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{C1}, —C(═O)OR^{C1}, —C(═O)N(R^{C1})₂, or a nitrogen protecting group, or R^{C} and R^{B} are joined to form a substituted or unsubstituted heterocyclic ring;
each instance of R^{C1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or an oxygen protecting group when attached to an oxygen atom, or about two instances of R^{C1} are joined to form a substituted or unsubstituted heterocyclic ring;
each instance of R^{D} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{D1}, —N(R^{D1})₂, —SR^{D1}, —CN, —SCN, —C(═NR^{D1})R^{D1}, —C(═NR^{D1})OR^{D1}, —C(═NR^{D1})N(R^{D1})₂, —C(═O)R^{D1}, —C(═O)OR^{D1}, —C(═O)N(R^{D1})₂, —NO₂, —NR^{D1}C(═O)R^{D1}, —NR^{D1}C(═O)OR^{D1}, —NR^{D1}C(═O)N(R^{D1})₂, —OC(═O)R^{D1}, —OC(═O)OR^{D1}, or —OC(═O)N(R^{D1})₂, or about two instances of R^{D} are joined to form a substituted or unsubstituted carbocyclic, substituted or unsubstituted heterocyclic, substituted or unsubstituted aryl, or substituted or unsubstituted heteroaryl ring;
each instance of R^{D1} is independently hydrogen, substituted or unsubstituted acyl substituted or unsubstituted alkyl, substituted or unsubstituted alkenyl, substituted or unsubstituted alkynyl, substituted or to carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two instances of R^{D1} are joined to form a substituted or unsubstituted heterocyclic ring;
R^{E} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{E1}, —C(═O)OR^{E1}, —C(═O)N(R^{E1})₂, or a nitrogen protecting group;
each instance of R^{E1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or an oxygen protecting group when attached to an oxygen atom, or about two instances of R^{E1} are joined to form a substituted or unsubstituted heterocyclic ring;
each instance of R^{F} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{F1}, —N(R^{F1})₂, —SR^{F1}, —CN, —SCN, —C(═NR^{F1})R^{F1}, —C(═NR^{F1})OR^{F1}, —C(═NR^{F1})N(R^{F1})₂, —C(═O)R^{F1}, —C(═O)OR^{F1}, —C(═O)N(R^{F1})₂, —NO₂, —NR^{F1}C(═O)R^{F1}, —NR^{F1}C(═O)OR^{F1}, —NR^{F1}C(═O)N(R^{F1})₂, —OC(═O)R^{F1}, —OC(═O)OR^{F1}, or —OC(═O)N(R^{F1})₂, or about two instances of R^{F} are joined to form a substituted or unsubstituted carbocyclic, substituted or unsubstituted heterocyclic, substituted or unsubstituted aryl, or substituted or unsubstituted heteroaryl ring;
each instance of R^{F1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two instances of R^{F1} are joined to form a substituted or unsubstituted heterocyclic ring;
a is 0, 1, 2, 3, 4, or 5;
d is 0, 1, or 2;
f is 0, 1, 2, 3 or 4; and
g is 0, 1, 2, or 3.

In certain embodiments, the compound of Formula (V) is of Formula (V-A):

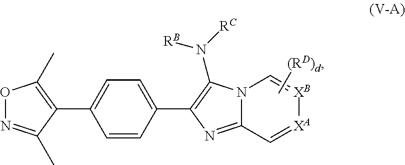

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-B):

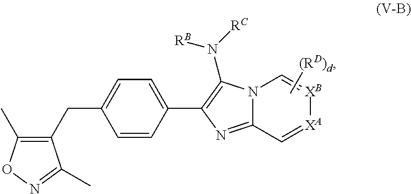

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-C):

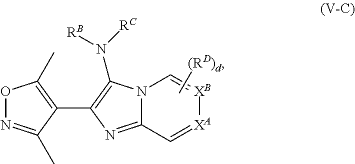

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-D):

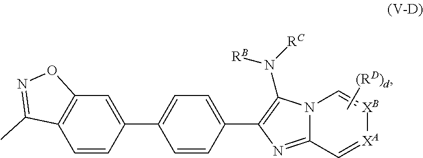

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-E):

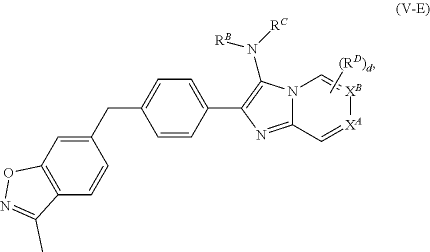

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-F):

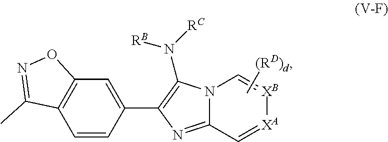

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-G):

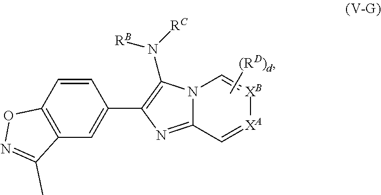

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-H):

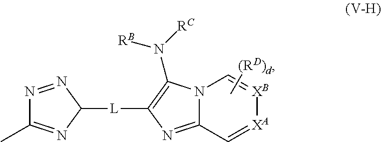

or a pharmaceutically acceptable salt, solvate, hydrate, polymorph, co-crystal, tautomer, stereoisomer, isotopically labeled derivative, or prodrug thereof.

In certain embodiments, the compound of Formula (V) is of Formula (V-J):

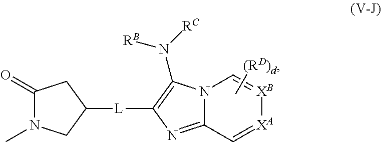

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

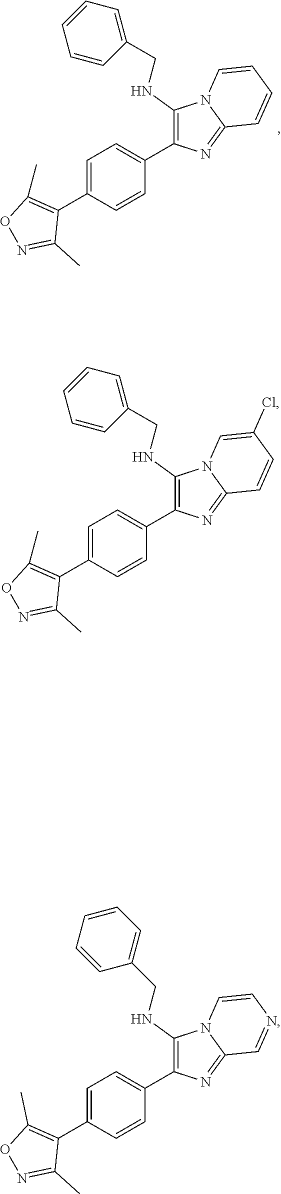 [SMILES: Cc1noc(C)c1-c1ccc(-c2nc3ccccn3c2NCc2ccccc2)cc1 ; "," ; Cc1noc(C)c1-c1ccc(-c2nc3ccc(Cl)cn3c2NCc2ccccc2)cc1 ; Cc1noc(C)c1-c1ccc(-c2nc3cnccn3c2NCc2ccccc2)cc1]

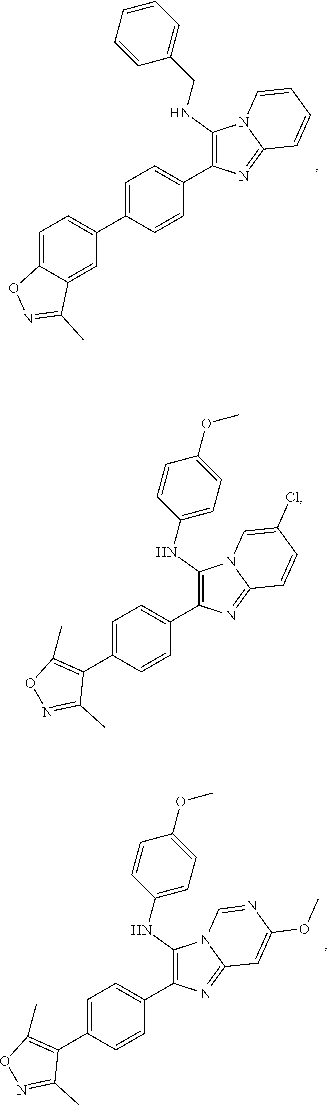 [SMILES: Cc1noc2ccc(-c3ccc(-c4nc5ccccn5c4NCc4ccccc4)cc3)cc12 ; "," ; COc1ccc(Nc2c(-c3ccc(-c4c(C)noc4C)cc3)nc3ccc(Cl)cn23)cc1 ; COc1ccc(Nc2c(-c3ccc(-c4c(C)noc4C)cc3)nc3cc(OC)ncn23)cc1 ; ","]

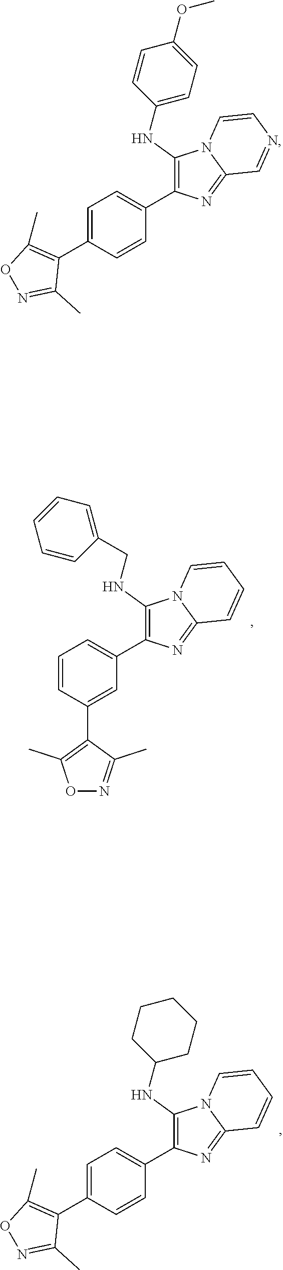 [SMILES: COc1ccc(Nc2c(-c3ccc(-c4c(C)noc4C)cc3)nc3cnccn23)cc1 ; Cc1noc(C)c1-c1cccc(-c2nc3ccccn3c2NCc2ccccc2)c1 ; "," ; Cc1noc(C)c1-c1ccc(-c2nc3ccccn3c2NC2CCCCC2)cc1 ; ","]

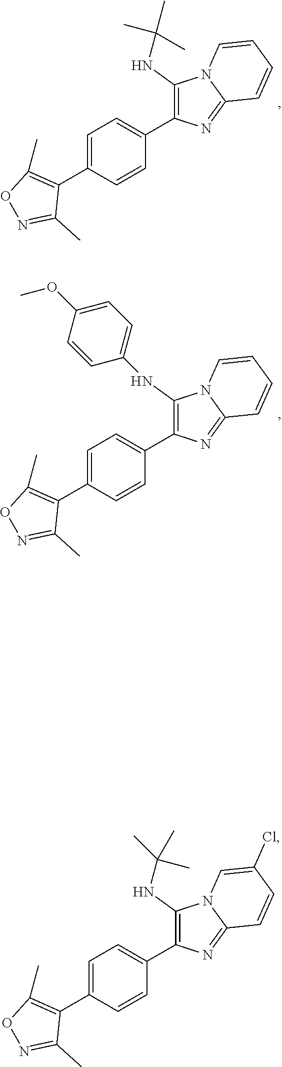 [SMILES: Cc1noc(C)c1-c1ccc(-c2nc3ccccn3c2NC(C)(C)C)cc1 ; "," ; COc1ccc(Nc2c(-c3ccc(-c4c(C)noc4C)cc3)nc3ccccn23)cc1 ; "," ; Cc1noc(C)c1-c1ccc(-c2nc3ccc(Cl)cn3c2NC(C)(C)C)cc1]

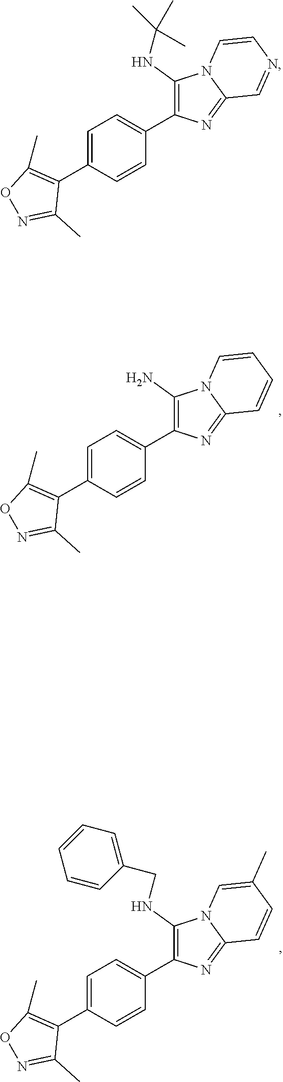 [SMILES: Cc1noc(C)c1-c1ccc(-c2nc3cnccn3c2NC(C)(C)C)cc1 ; Cc1noc(C)c1-c1ccc(-c2nc3ccccn3c2N)cc1 ; "," ; Cc1ccc2nc(-c3ccc(-c4c(C)noc4C)cc3)c(NCc3ccccc3)n2c1 ; ","]

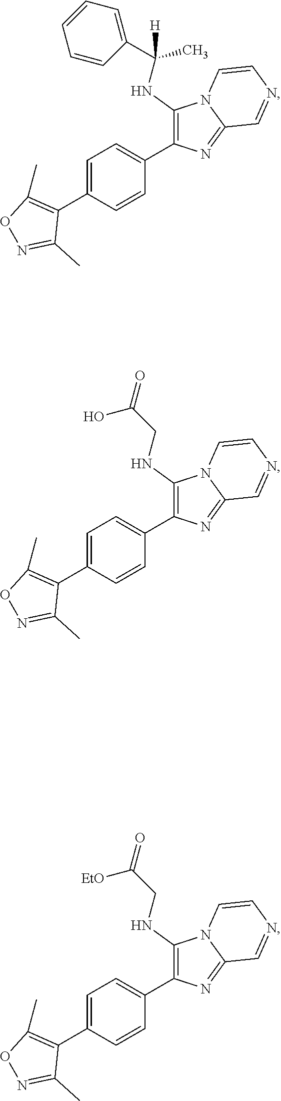 [SMILES: Cc1noc(C)c1-c1ccc(-c2nc3cnccn3c2N[C@@H](C)c2ccccc2)cc1 ; Cc1noc(C)c1-c1ccc(-c2nc3cnccn3c2NCC(=O)O)cc1 ; CCOC(=O)CNc1c(-c2ccc(-c3c(C)noc3C)cc2)nc2cnccn12]

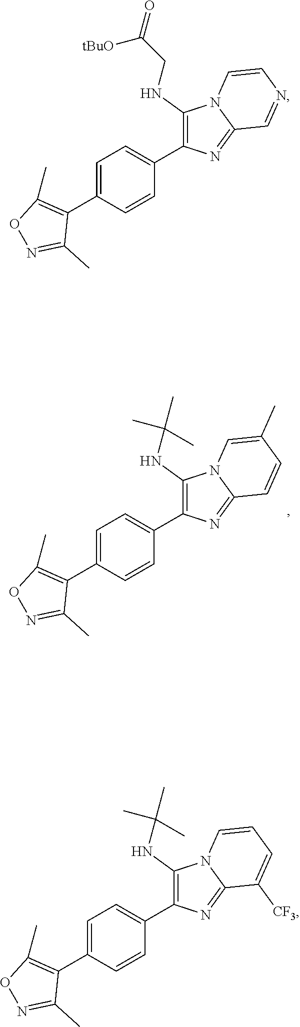 [SMILES: Cc1noc(C)c1-c1ccc(-c2nc3cnccn3c2NCC(=O)OC(C)(C)C)cc1 ; Cc1ccc2nc(-c3ccc(-c4c(C)noc4C)cc3)c(NC(C)(C)C)n2c1 ; "," ; Cc1noc(C)c1-c1ccc(-c2nc3c(C(F)(F)F)cccn3c2NC(C)(C)C)cc1]

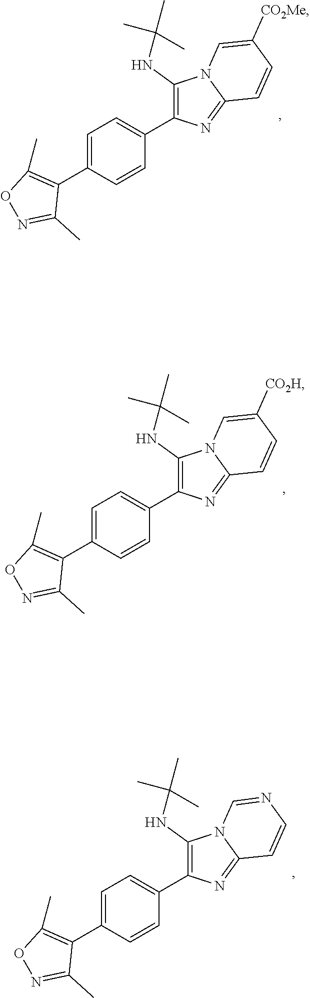 [SMILES: COC(=O)c1ccc2nc(-c3ccc(-c4c(C)noc4C)cc3)c(NC(C)(C)C)n2c1 ; "," ; Cc1noc(C)c1-c1ccc(-c2nc3ccc(C(=O)O)cn3c2NC(C)(C)C)cc1 ; "," ; Cc1noc(C)c1-c1ccc(-c2nc3ccncn3c2NC(C)(C)C)cc1 ; ","]

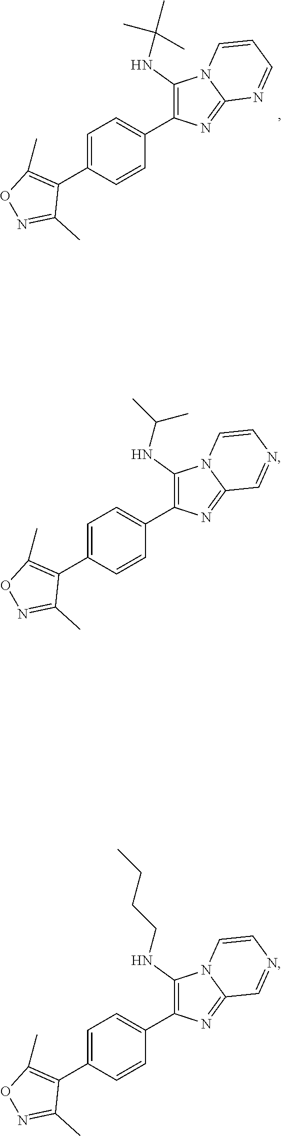 [SMILES: Cc1noc(C)c1-c1ccc(-c2nc3ncccn3c2NC(C)(C)C)cc1 ; "," ; Cc1noc(C)c1-c1ccc(-c2nc3cnccn3c2NC(C)C)cc1 ; CCCCNc1c(-c2ccc(-c3c(C)noc3C)cc2)nc2cnccn12]

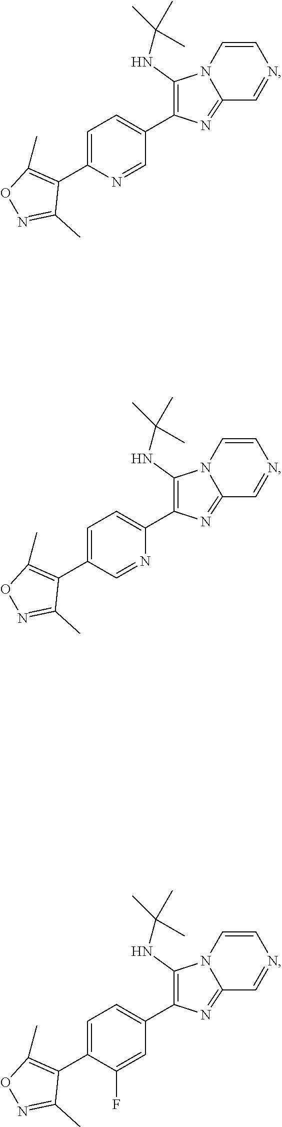 [SMILES: Cc1noc(C)c1-c1ccc(-c2nc3cnccn3c2NC(C)(C)C)cn1 ; Cc1noc(C)c1-c1ccc(-c2nc3cnccn3c2NC(C)(C)C)nc1 ; Cc1noc(C)c1-c1ccc(-c2nc3cnccn3c2NC(C)(C)C)cc1F]

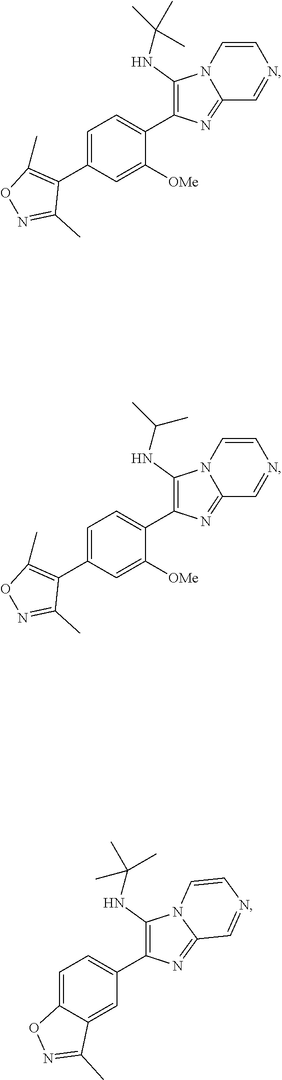 [SMILES: COc1cc(-c2c(C)noc2C)ccc1-c1nc2cnccn2c1NC(C)(C)C ; COc1cc(-c2c(C)noc2C)ccc1-c1nc2cnccn2c1NC(C)C ; Cc1noc2ccc(-c3nc4cnccn4c3NC(C)(C)C)cc12]

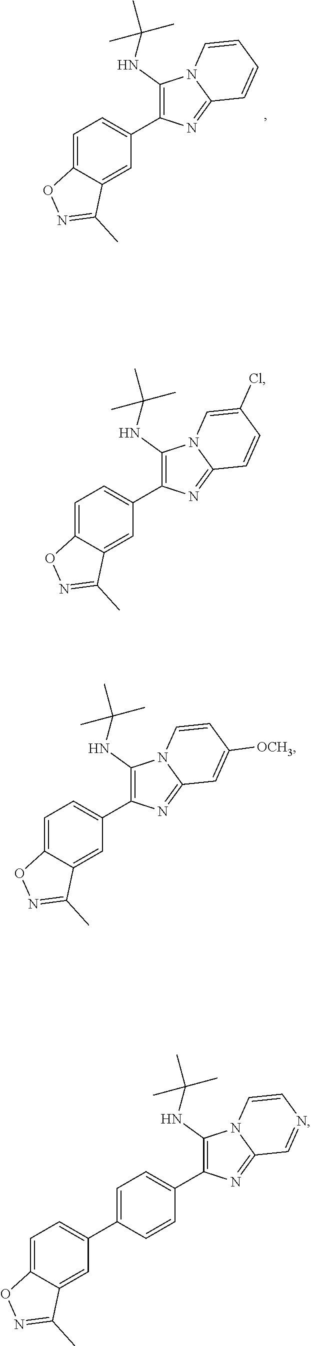 [SMILES: Cc1noc2ccc(-c3nc4ccccn4c3NC(C)(C)C)cc12 ; "," ; Cc1noc2ccc(-c3nc4ccc(Cl)cn4c3NC(C)(C)C)cc12 ; COc1ccn2c(NC(C)(C)C)c(-c3ccc4onc(C)c4c3)nc2c1 ; Cc1noc2ccc(-c3ccc(-c4nc5cnccn5c4NC(C)(C)C)cc3)cc12]

or a pharmaceutically acceptable salt, solvate, hydrate, polymorph, co-crystal, tautomer, stereoisomer, isotopically labeled derivative, or prodrug thereof.

### Compounds of Formula (VI)

In certain embodiments, the bromodomain inhibitor is an inhibitor disclosed in international PCT Publication No. WO 2015/117055, which is incorporated herein by reference.

In certain embodiments, the bromodomain inhibitor is of Formula (VI):

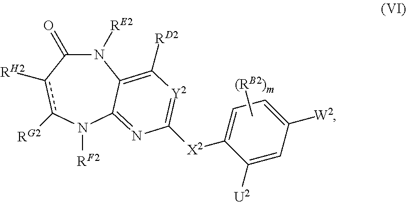

or a pharmaceutically acceptable salt, solvate, hydrate, polymorph, co-crystal, tautomer, stereoisomer, isotopically labeled derivative, or prodrug thereof, wherein:
[symbol] is a single or double bond;
W² is —C(═O)OR^{W2}, —C(═O)N(R^{W2})₂, —S(═O)OR^{W2}, —S(═O)N(R^{W2})₂, —S(═O)₂OR^{W2}, —S(═O)₂N(R^{W2})₂,

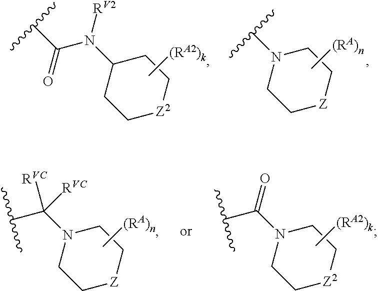

each instance of R^{W2} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, an oxygen protecting group when attached to an oxygen atom, or a nitrogen protecting group when attached to a nitrogen atom, or two instances of R^{W2} are joined to form a substituted or unsubstituted heterocyclic or substituted or unsubstituted heteroaryl ring;
R^{V2} is hydrogen, substituted or unsubstituted C₁₋₆ alkyl, or a nitrogen protecting group;
R^{VC} is hydrogen, halogen, or substituted or unsubstituted C₁₋₆ alkyl;
U² is R^{B2} or —OR^{C2};
X² is —O—, —S—, —N(R^{X2})—, or —C(R^{X2})₂—, wherein each instance of R^{X2} is independently hydrogen, halogen, substituted or unsubstituted C₁₋₆ alkyl, or a nitrogen protecting group when attached to a nitrogen atom;
Y² is N or CR^{D2};
Z² is —O—, —N(R^{Z2})— or —C(R^{Z2})₂—, wherein each instance of R^{Z2} is independently hydrogen, halogen, substituted, or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl or a nitrogen protecting group when attached to a nitrogen atom, or about two instances of R^{Z2} are joined to form a substituted or unsubstituted carbocyclic or substituted or unsubstituted heterocyclic ring;
each instance of R^{A2} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A2a}, —N(R^{A2a})₂, —SR^{A2a}, —CN, —SCN, —C(═NR^{A2a})R^{A2a}, —C(═NR^{A2a})OR^{A2a}, —C(═NR^{A2a})N(R^{A2a})₂, —C(═O)R^{A2a}, —C(═O)OR^{A2a}, —C(═O)N(R^{A2a})₂, —NO₂, —NR^{A2a}C(═O)R^{A2a}, —NR^{A2a}C(═O)OR^{A2a}, —NR^{A2a}C(═O)N(R^{A2a})₂, —OC(═O)R^{A2a}, —OC(═O)OR^{A2a}, or —OC(═O)N(R^{A2a})₂, wherein each instance of R^{A2a} is independently hydrogen, substituted or unsubstituted acyl, substituted or unsubstituted substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{A2a} groups are joined to form a substituted or unsubstituted heterocyclic or substituted or unsubstituted heteroaryl ring;
k is 0, 1, 2, 3, 4, 5, 6, 7, 8, or 9;
each instance of R^{B2} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{B2a}, ——N(R^{B2a})₂, —SR^{B2a}, —CN, —SCN, —C(═NR^{B2a})R^{B2a}, —C(═NR^{B2a})OR^{B2a}, —C(═NR^{B2a})N(R^{B2a})₂, —C(═O)R^{B2a}, —C(═O)OR^{B2a}, —C(═O)N(R^{B2a})₂, —NO₂, —NR^{B2a}C(═O)R^{B2a}, —NR^{B2a}C(═O)OR^{B2a}, —NR^{B2a}C(═O)N(R^{B2a})₂, —OC(═O)R^{B2a}, —OC(═O)OR^{B2a}, or —OC(═O)N(R^{B2a})₂, wherein each instance of R^{B2a} is independently hydrogen, substituted or unsubstituted acyl, substituted or unsubstituted alkyl, substituted or unsubstituted alkenyl, substituted or unsubstituted alkynyl, substituted or unsubstituted carbocyclyl substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B2a} groups are joined to form a substituted or unsubstituted heterocyclic or substituted or unsubstituted heteroaryl ring;
m is 0, 1, 2, or 3;
R^{C2} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an oxygen protecting group;
each instance of R^{D2} is independently hydrogen, halogen, substituted or unsubstituted alkyl, substituted or unsubstituted alkenyl, substituted or unsubstituted alkynyl, substituted or unsubstituted carbocyclyl substituted or unsubstituted heterocyclyl, substituted or unsubstituted aryl, substituted or unsubstituted heteroaryl, —OR^{D2a}, —N(R^{D2a})₂, —SR^{D2a}, —CN, —SCN, —C(═NR^{D2a})R^{D2a}, —C(═O)OR^{D2a}, —C(═NR^{D2a})N(R^{D2a})₂, —C(═O)R^{D2a}, —C(═O)OR^{D2a}, —C(═O)N(R^{D2a})₂, —NO₂, —NR^{D2}C(═O)R^{D2a}, —NR^{D2a}C(═O)OR^{D2a}, —NR^{D2a}C(═O)N(R^{D2a})₂, —OC(═O)R^{D2a}, —OC(═O)OR^{D2a}, or —OC(═O)N(R^{D2a})₂, wherein each instance of R^{D2a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{D2}a groups are joined to form a substituted or unsubstituted heterocyclic or substituted or unsubstituted heteroaryl ring;
n is 0, 1, or 2;
R^{E2} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group;
R^{F2} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group;
R^{G2} is hydrogen, halogen, or substituted or unsubstituted C₁₋₆ alkyl; and
R^{H2} is hydrogen, halogen, or substituted or unsubstituted C₁₋₆ alkyl;
or R^{G2} and R^{H2} are joined to form a substituted or unsubstituted phenyl ring.

In certain embodiments, the bromodomain inhibitor of Formula (VI) is of Formula (VI-A):

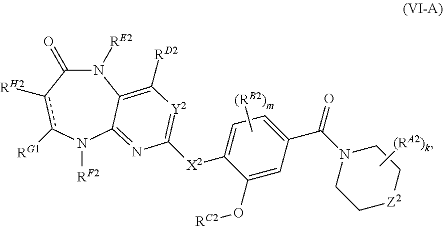

or a pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (VI) is of Formula (VI-B):

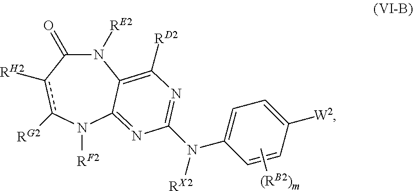

or a pharmaceutically acceptable salt, solvate, hydrate, polymorph, co-crystal, tautomer, stereoisomer, isotopically labeled derivative, or prodrug thereof.

In certain embodiments, the bromodomain inhibitor of Formula (VI) is of Formula (VI-C):

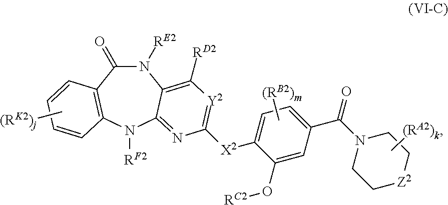

or a pharmaceutically acceptable salt, solvate, hydrate, polymorph, co-crystal, tautomer, stereoisomer, isotopically labeled derivative, or prodrug thereof, wherein:
each instance of R^{K2} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{K2a}, —N(R^{K2a})₂, —SR^{K2a}, —CN, —SCN, —C(═NR^{2a})R^{K2a}, —C(═NR^{K2a})OR^{K2a}, —C(═NR^{K2a})N(R^{K2a})₂, —C(═O)R^{K2a}, —C(═O)OR^{K2a}, —C(═O)N(R^{K2a})₂, —NO₂, —NR^{K2a}C(═O)R^{K2a}, —NR^{K2a}C(═O)OR^{K2a}, —NR^{K2a}C(═O)N(R^{K2a})₂, —OC(═O)R^{K2a}, —OC(═O)OR^{K2a}, or —OC(═O)N(R^{K2a})₂, wherein each instance of R^{K2a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{K2a} groups are joined to form a substituted or unsubstituted heterocyclic or substituted or unsubstituted heteroaryl ring; and
j is 0, 1, 2, 3, or 4.

In certain embodiments, the bromodomain inhibitor of Formula (VI) is of Formula (VI-D):

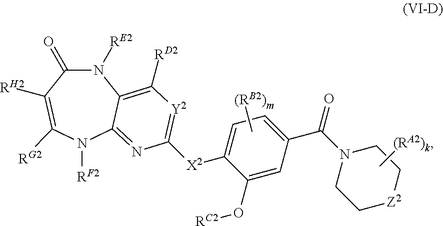

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

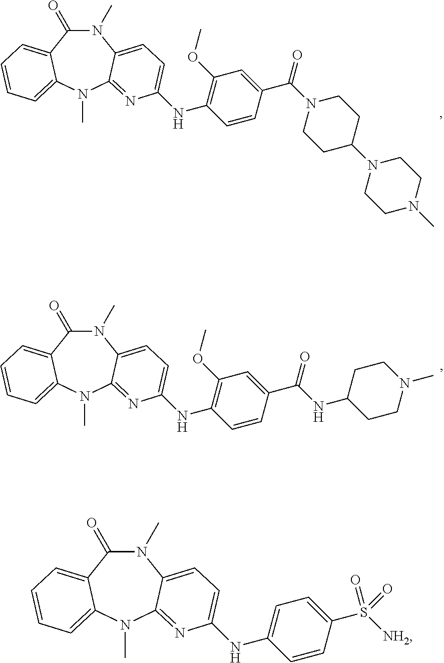 [SMILES: COc1cc(C(=O)N2CCC(N3CCN(C)CC3)CC2)ccc1Nc1ccc2c(n1)N(C)c1ccccc1C(=O)N2C ; COc1cc(C(=O)NC2CCN(C)CC2)ccc1Nc1ccc2c(n1)N(C)c1ccccc1C(=O)N2C ; CN1C(=O)c2ccccc2N(C)c2nc(Nc3ccc(S(N)(=O)=O)cc3)ccc21]

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

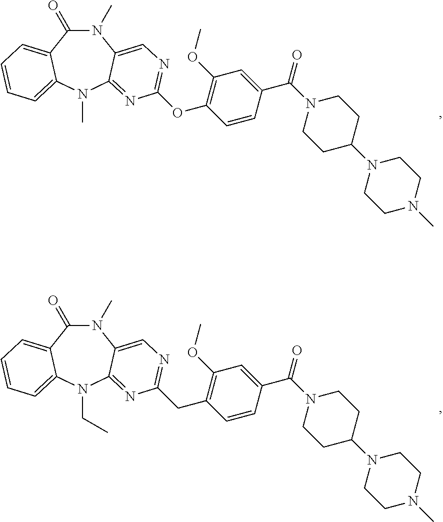 [SMILES: COc1cc(C(=O)N2CCC(N3CCN(C)CC3)CC2)ccc1Oc1ncc2c(n1)N(C)c1ccccc1C(=O)N2C ; CCN1c2ccccc2C(=O)N(C)c2cnc(Cc3ccc(C(=O)N4CCC(N5CCN(C)CC5)CC4)cc3OC)nc21]

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

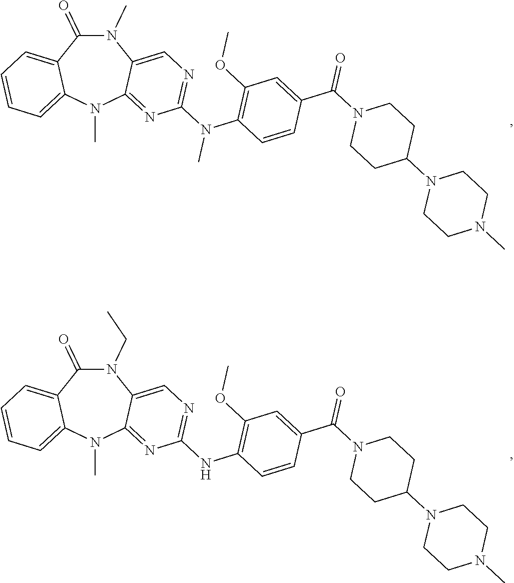 [SMILES: COc1cc(C(=O)N2CCC(N3CCN(C)CC3)CC2)ccc1N(C)c1ncc2c(n1)N(C)c1ccccc1C(=O)N2C ; "," ; CCN1C(=O)c2ccccc2N(C)c2nc(Nc3ccc(C(=O)N4CCC(N5CCN(C)CC5)CC4)cc3OC)ncc21 ; ","]

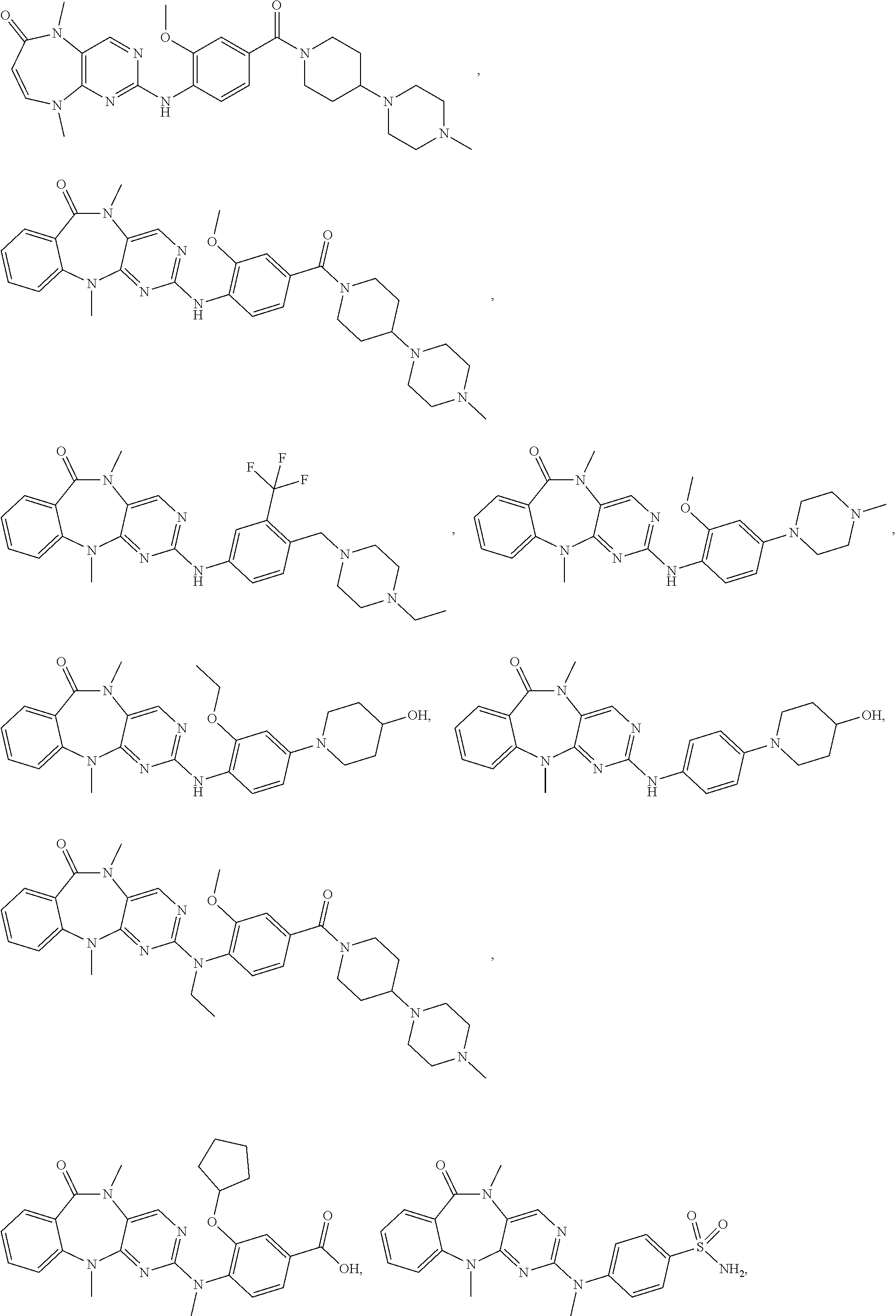

or a pharmaceutically acceptable salt, solvate, hydrate, polymorph, co-crystal, tautomer, stereoisomer, isotopically labeled derivative, or prodrug thereof.

### Compounds of Formula (VII)

In certain embodiments, the bromodomain inhibitor is an inhibitor disclosed in international PCT Publication No. WO 2015/117083, which is incorporated herein by reference.

In certain embodiments, the bromodomain inhibitor is of Formula (VII):

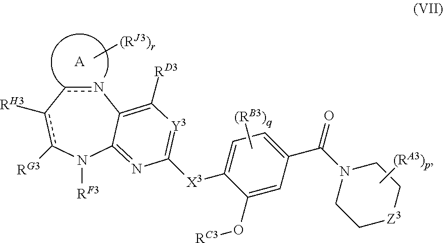

or a pharmaceutically acceptable salt, solvate, hydrate, polymorph, co-crystal, tautomer, stereoisomer, isotopically labeled derivative, or prodrug thereof, wherein:
each instance of [symbol] is independently a single or double bond;
X³ is —O—, —S—, —N(R^{X3})—, or —C(R^{X3})₂—, wherein each instance of R^{X3} is independently hydrogen, halogen, substituted or unsubstituted C₁₋₆ alkyl, or a nitrogen protecting group when attached to a nitrogen atom;
Y³ is N or CR^{Y3}, wherein R^{Y3} is hydrogen, halogen, or substituted or unsubstituted alkyl;
Z³ is —O—, —N(R^{Z3})— or —C(R^{Z3})₂—, wherein each instance of R^{Z3} is independently hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group when attached to a nitrogen atom, or about two instances of R^{Z3} are joined to form a substituted or unsubstituted carbocyclic or substituted or unsubstituted heterocyclic ring;
each instance of R^{A3} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{A3a}, —N(R^{A3a})₂, —SR^{A3a}, —CN, —SCN, —C(═NR^{A3a})R^{A3a}, —C(═NR^{A3a})OR^{A3a}, —C(═NR^{A3a})N(R^{A3a})₂, —C(═O)R^{A3a}, —C(═O)OR^{A3a}, —C(═O)N(R^{A3a})₂, —NO₂, —NR^{A3a}C(═O)R^{A3a}, —NR^{A3a}C(═O)OR^{A3a}, —NR^{A3a}C(═O)N(R^{A3a})₂, —OC(═O)R^{A3a}, —OC(═O)OR^{A3a}, or —OC(═O)N(R^{A3a})₂, wherein each instance of R^{A3a} is independently hydrogen, substituted or unsubstituted acyl, substituted or unsubstituted substituted or unsubstituted alkenyl, substituted or unsubstituted alkenyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{A3a} groups are joined to form a substituted or unsubstituted heterocyclic or substituted or unsubstituted heteroaryl ring;
p is 0, 1, 2, 3, 4, 5, 6, 7, or 8;
each instance of R^{B3} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{B3a}, ——N(R^{B3a})₂, —SR^{B3a}, —CN, —SCN, —C(═NR^{B3a})R^{B3a}, —C(═NR^{B3a})OR^{B3a}, —C(═NR^{B3a})N(R^{B3a})₂, —C(═O)R^{B3a}, —C(═O)OR^{3a}, —C(═O)N(R^{B3a})₂, —NO₂, —NR^{B3a}C(═O)R^{B3a}, —NR^{B3a}C(═O)OR^{B3a}, —NR^{B3a}C(═O)N(R^{B3a})₂, —O(═O)R^{B3a}, —OC(═O)OR^{B3a}, or —OC(═O)N(R^{B3a})₂, wherein each instance of R^{B3a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B3a} groups are joined to form a substituted or unsubstituted heterocyclic or substituted or unsubstituted heteroaryl ring;
q is 0, 1, 2, or 3;
R^{C3} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an oxygen protecting group;
R^{D3} is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{D3a}, —N(R^{D3a})₂, —SR^{D3a}, —CN, —SCN, —C(═NR^{D3a})R^{D3a}, —C(═NR^{D3a})OR^{D3a}, —C(═NR^{D3a})N(R^{D3a})₂, —C(═O)R^{D3a}, —C(═O)OR^{D3a}, —C(═O)N(R^{D3a})₂, —NO₂, —NR^{D3a}C(═O)R^{D3a}, —NR^{D3a}C(═O)OR^{D3a}, —NR^{D3a}C(═O)N(R^{D3a})₂, —OC(═O)R^{D3a}, —OC(═O)OR^{D3a}, or —OC(═O)N(R^{D3a})₂, wherein each instance of R^{D3a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{D3a} groups are joined to form a substituted or unsubstituted heterocyclic or substituted or unsubstituted heteroaryl ring;
Ring A is substituted or unsubstituted, 5- to 6-membered, monocyclic, heterocyclic or heteroaryl ring;
each instance of R^{J3} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{J3a}, —N(R^{J3a})₂, —SR^{J3a}, —CN, —SCN, —C(═NR^{J3a})R^{J3a}, —C(═NR^{J3a})OR^{J3a}, —C(═NR^{J3a})N(R^{J3a})₂, —C(═O)R^{J3a}, —(═O)OR^{J3a}, —C(═O)N(R^{J3a})₂, —NO₂, —NR^{J3a}C(═O)R^{J3a}, —NR^{J3a}C(═O)OR^{J3a}, —NR^{J3a}C(═O)N(R^{J3a})₂, —OC(═O)R^{J3a}, —OC(═O)OR^{J3a}, —OC(═O)N(R^{J3a})₂, or a nitrogen protecting group when attached to a nitrogen atom, wherein each instance of R^{J3a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{J3a} groups are joined to form a substituted or unsubstituted heterocyclic or substituted or unsubstituted heteroaryl ring;
r is 0, 1, 2, 3, 4, 5, 6, 7, or 8;
R^{F3} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group;
R^{G3} is hydrogen, halogen, or substituted or unsubstituted C₁₋₆ alkyl; and
R^{H3} is hydrogen, halogen, or substituted or unsubstituted alkyl;
or R^{G3} and R^{H3} are joined to form a substituted or unsubstituted phenyl ring.

In certain embodiments, the bromodomain inhibitor of Formula (VII) is of Formula (VII-A):

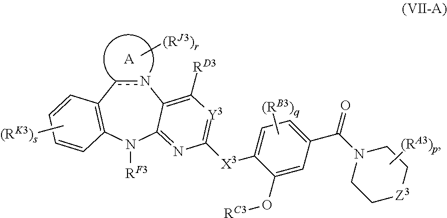

or a pharmaceutically acceptable salt, solvate, hydrate, polymorph, co-crystal, tautomer, stereoisomer, isotopically labeled derivative, or prodrug thereof, wherein:
each instance of R^{K3} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{K3a}, —N(R^{K3}a)₂, —SR^{K3a}, —CN, —SCN, —C(═NR^{K3a})R^{K3a}, —C(═NR^{K3a})OR^{K3a}, —C(═NR^{K3a})N(R^{K3a})₂, —C(═O)R^{K3a}, —C(═O)OR^{K3a}, —C(═O)N(R^{K3a})₂, —NO₂, —NR^{K3a}C(═O)R^{K3a}, —NR^{K3a}C(═O)OR^{K3a}, —NR^{K3a}C(═O)N(R^{K3a})₂, —OC(═O)R^{K3a}, —OC(═O)OR^{K3a}, or —OC(═O)N(R^{K3a})₂, wherein each instance of R^{K3a} is independently hydrogen, substituted or unsubstituted acyl, substituted or unsubstituted substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{K3a} groups are joined to form a substituted or unsubstituted heterocyclic or substituted or unsubstituted heteroaryl ring; and
s is 0, 1, 2, 3, or 4.

In certain embodiments, a compound described herein is of Formula (III-B):

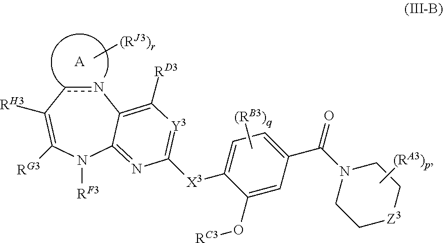

or a pharmaceutically acceptable salt, solvate, hydrate, polymorph, co-crystal, tautomer, stereoisomer, isotopically labeled derivative, or prodrug thereof.

In certain embodiments, a compound described herein is of Formula (III-C):

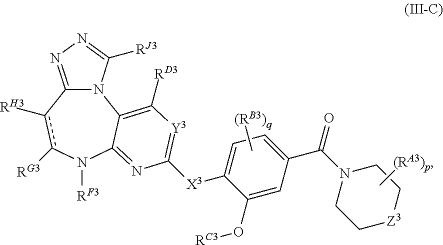

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

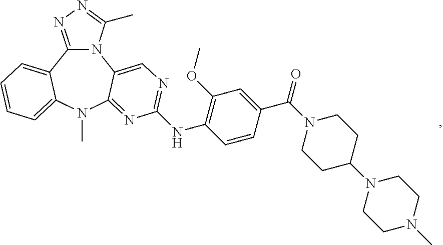 [SMILES: COc1cc(C(=O)N2CCC(N3CCN(C)CC3)CC2)ccc1Nc1ncc2c(n1)N(C)c1ccccc1-c1nnc(C)n1-2]

or a pharmaceutically acceptable salt, solvate, hydrate, polymorph, co-crystal, tautomer, stereoisomer, isotopically labeled derivative, or prodrug thereof.

Compounds of Formula (VIII)

In certain embodiments, the bromodomain inhibitor is an inhibitor disclosed in international PCT Publication No. WO 2015/117055, which is incorporated herein by reference.

In certain embodiments, the bromodomain inhibitor is of Formula (VIII):

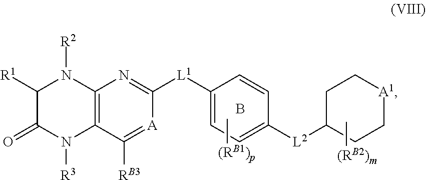

or pharmaceutically acceptable salt, solvate, hydrate, polymorph, co-crystal, tautomer, stereoisomer, isotopically labeled derivative, or prodrug thereof, wherein:
A is ═N— or ═C(R^{B4})—;
A¹ is —N(R⁴)— or —C(R⁴)₂—;
R¹ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R² and R³ are each independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{D1}, —C(═O)OR^{D1}, —C(═O)N(R^{D1})₂, or a nitrogen protecting group, wherein each instance of R^{D1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{D1} groups are joined to form a substituted or unsubstituted heterocyclic or substituted or unsubstituted heteroaryl ring, or a nitrogen protecting group when attached to a nitrogen atom;
R⁴ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{D1}, —C(═O)OR^{D1}, or —C(═O)N(R^{D1})₂, wherein each instance of R^{D1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{D1} groups are joined to form a substituted or unsubstituted heterocyclic or substituted or unsubstituted heteroaryl ring, or a nitrogen protecting group when attached to a nitrogen atom;
each instance of R^{B1} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{B1a}, —N(R^{B1a})₂, —SR^{B1a}, —CN, —SCN, —C(═NR^{B1a})R^{B1a}, —C(═NR^{B1a})OR^{B1a}, —C(═NR^{B1a})N(R^{B1a})₂, —C(═O)R^{B1a}, —C(═O)OR^{B1a}, —C(═O)N(R^{B1a})₂, —NO₂, —NR^{B1a}C(═O)R^{B1a}, —NR^{B1a}C(═O)OR^{B1a}, —NR^{B1a}C(═O)N(R^{B1a})₂, —OC(═O)R^{B1a}, —OC(═O)OR^{B1a}, or —OC(═O)N(R^{B1a})₂, wherein each instance of R^{B1a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B1a} groups are joined to form a substituted or unsubstituted heterocyclic or substituted or unsubstituted heteroaryl ring;
each instance of R^{B2} is independently hydrogen, halogen, substituted or unsubstituted alkyl, substituted or unsubstituted alkenyl, substituted or unsubstituted substituted or unsubstituted carbocyclyl, substituted or unsubstituted heterocyclyl, substituted or unsubstituted aryl, substituted or unsubstituted heteroaryl, —OR^{B2a}, —N(R^{B2a})₂, —SR^{B2a}, —CN, —SCN, —C(═NR^{B2a})R^{B2a}, —C(═NR^{B2a})OR^{B2a}, —C(═NR^{B2a})N(R^{B2a})₂, —C(═O)R^{B2a}, —C(═O)OR^{B2a}, —C(═O)N(R^{B2a})₂, —NO₂, —NR^{B2a}C(═O)R^{B2a}, —NR^{B2a}C(═O)OR^{B2a}, —NR^{B2a}C(═O)N(R^{B2a})₂, —OC(═O)R^{B2a}, —OC(═O)OR^{B2a}, or —OC(═O)N(R^{B2a})₂, wherein each instance of R^{B2a} is independently hydrogen, substituted or unsubstituted acyl, substituted or unsubstituted alkyl, substituted or unsubstituted alkenyl, substituted or unsubstituted alkenyl, substituted or unsubstituted carbocyclyl, substituted or unsubstituted heterocyclyl, substituted or unsubstituted acyl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B2a} groups are joined to form a substituted or unsubstituted heterocyclic or substituted or unsubstituted heteroaryl ring;
each instance of R^{B3} is independently hydrogen, halogen, substituted or unsubstituted alkyl, substituted or unsubstituted alkenyl substituted or unsubstituted alkynyl, substituted or unsubstituted carbocyclyl, substituted or unsubstituted heterocyclyl, substituted or unsubstituted aryl, substituted or unsubstituted heteroaryl, —OR^{B3a}, —N(R^{B3a})₂, —SR^{B3a}, —CN, —SCN, —C(═NR^{B3a})R^{B3a}, —C(═NR^{B3a})OR^{B3a}, —C(═NR^{B3a})N(R^{B3a})₂, —C(═O)R^{B3a}, —C(═O)OR^{B3a}, —C(═O)N(R^{B3a})₂, —NO₂, —NR^{B3a}C(═O)R^{B3a}, —NR^{B3a}C(═O)OR^{B3a}, —NR^{B3a}C(═O)N(R^{B3a})₂e, —OC(═O)R^{B3a}, —OC(═O)OR^{B3a}, or —OC(═O)N(R^{B3a})₂, wherein each instance of R^{B3a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B3a} groups are joined to form a substituted or unsubstituted heterocyclic or substituted or unsubstituted heteroaryl ring;
each instance of R^{B4} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{B4a}, —N(R^{B4a})₂, —SR^{B4a}, —CN, —SCN, —C(═NR^{B4a})R^{B4a}, —C(═NR^{B4a})OR^{B4a}, —C(═NR^{B4a})N(R^{B4a})₂, —C(═O)R^{B4a}, —C(═O)OR^{B4a}, —C(═O)N(R^{B4a})₂, —NO₂, —NR^{B4a}C(═O)R^{B4a}, —NR^{B4a} C(═O)OR^{B4a}, —NR^{B4a}C(═O)N(R^{B4a})₂, —OC(═O)R^{B4a}, —OC(═O)OR^{B4a}, or —OC(═O)N(R^{B4a})₂, wherein each instance of R^{B4a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B4a} groups are joined to form a substituted or unsubstituted heterocyclic or substituted or unsubstituted heteroaryl ring;
m is 0 or an integer between 1 and 8, inclusive;
p is 0 or an integer between 1 and 4, inclusive;
each of L¹ and L² is independently a bond,

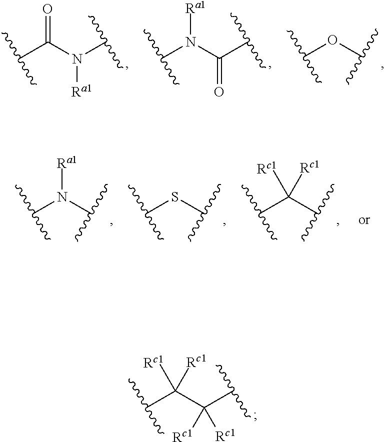

each instance of R^{a1} is independently hydrogen, substituted or unsubstituted alkyl, substituted or unsubstituted alkenyl, substituted or unsubstituted alkenyl, substituted or unsubstituted carbocyclyl, substituted or unsubstituted heterocyclyl, substituted or unsubstituted aryl, substituted or unsubstituted heteroaryl, or a nitrogen protecting group; or, if L¹ is

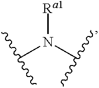

then R^{a1} of L¹ and one instance of R^{B1} that is ortho to L¹ are joined to form a substituted or unsubstituted heterocyclic ring or substituted or unsubstituted heteroaryl ring; and
each instance of R^{c1} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{c1a}, —N(R^{c1a})₂, —SR^{c1a}, —CN, —C(═O)R^{c1a}, —C(═O)OR^{c1a}, —C(═O)N(R^{c1a})₂, —NR^{c1a}C(═O)R^{c1a}, —NR^{c1a}C(═O)OR^{c1a}, —NR^{c1a}C(═O)N(R^{c1a})₂, —OC(═O)R^{c1a}, or —OC(═O)N(R^{c1a})₂, wherein each instance of R^{c1a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{c1a} groups are joined to form a substituted or unsubstituted heterocyclic or substituted or unsubstituted heteroaryl ring.

In certain embodiments, the compound of Formula (VIII) is of Formula (VIII-A):

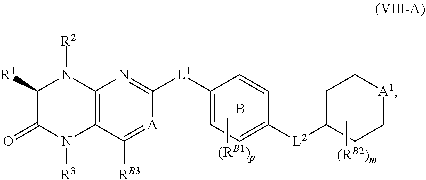

or a pharmaceutically acceptable salt, solvate, hydrate, polymorph, co-crystal, tautomer, stereoisomer, isotopically labeled derivative, or prodrug thereof.

In certain embodiments, the compound of Formula (VIII) is of Formula (VIII-B)

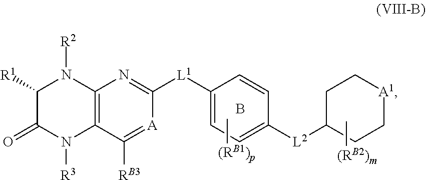

or a pharmaceutically acceptable salt, solvate, hydrate, polymorph, co-crystal, tautomer, stereoisomer, isotopically labeled derivative, or prodrug thereof.

In certain embodiments, the compound of Formula (VIII) is of Formula (VIII-C:

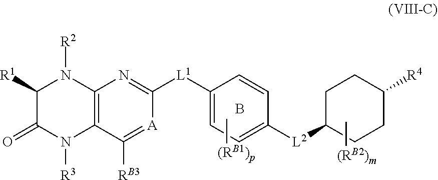

or a pharmaceutically acceptable salt, solvate, hydrate, polymorph, co-crystal, tautomer, stereoisomer, isotopically labeled derivative, or prodrug thereof.

In certain embodiments, the compound of Formula (VIII) is of Formula (VIII-D):

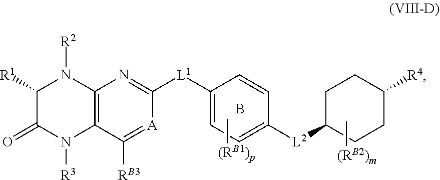

or a pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

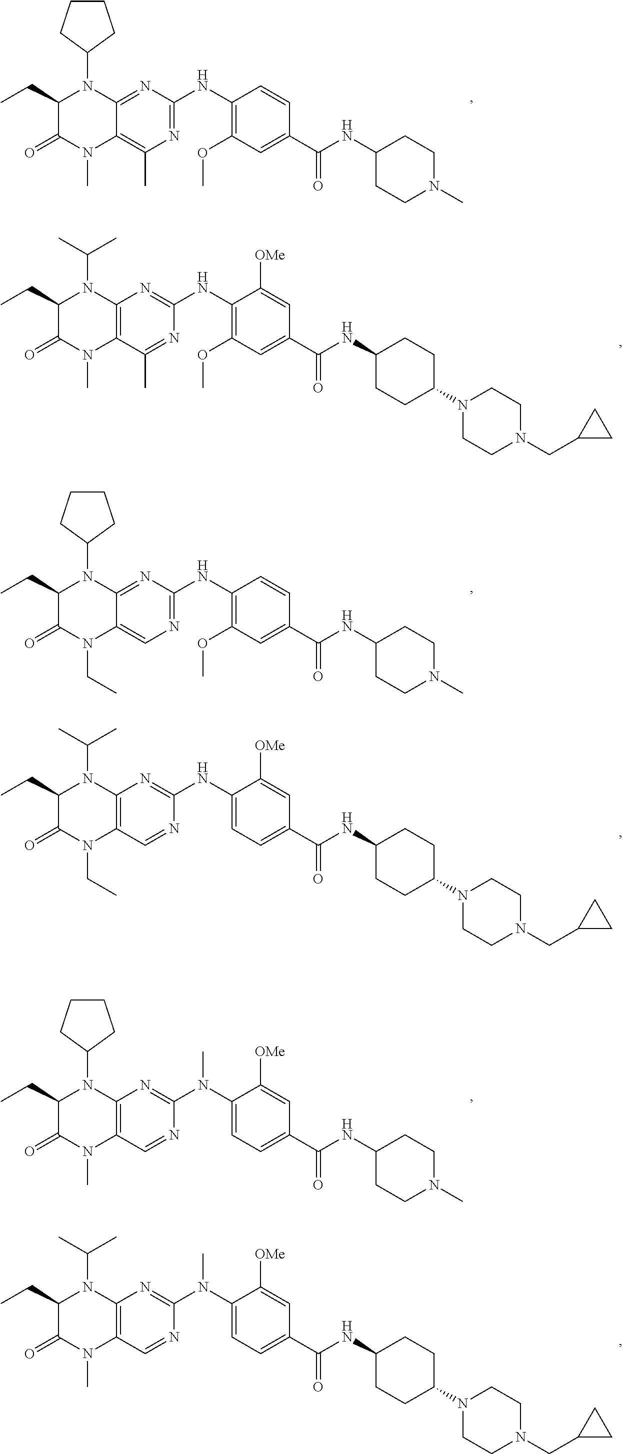

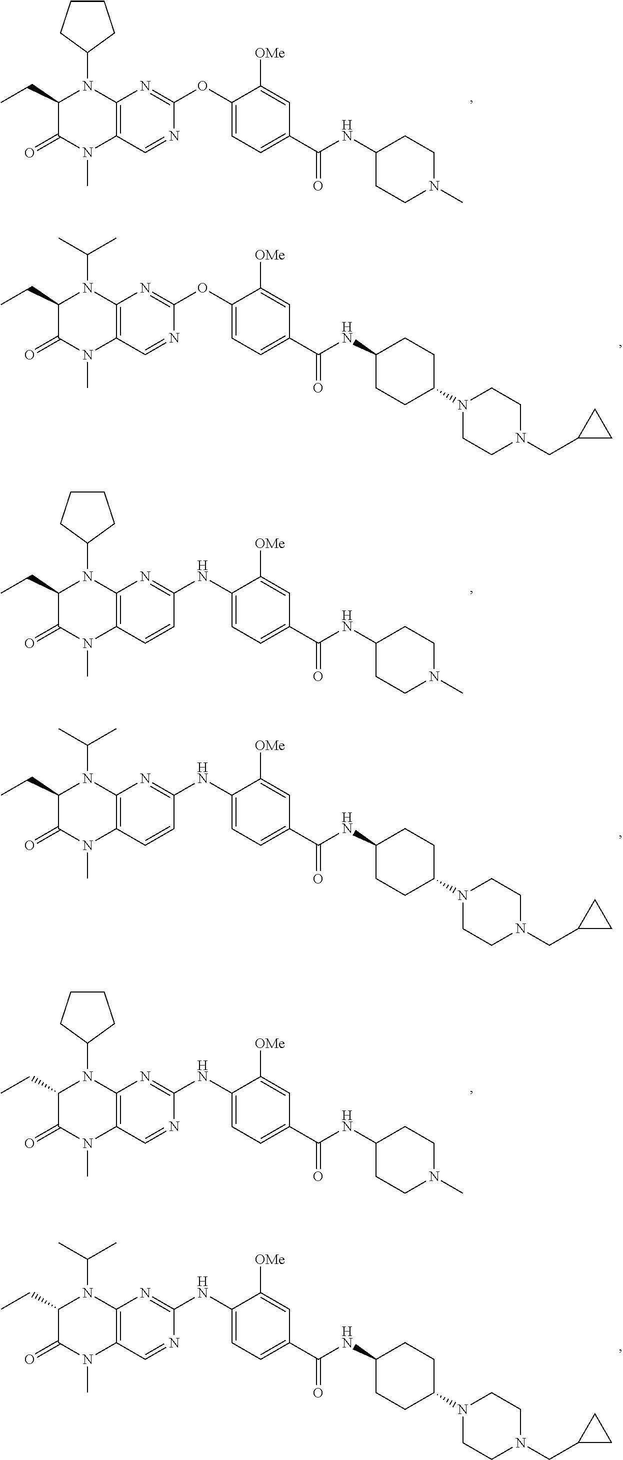

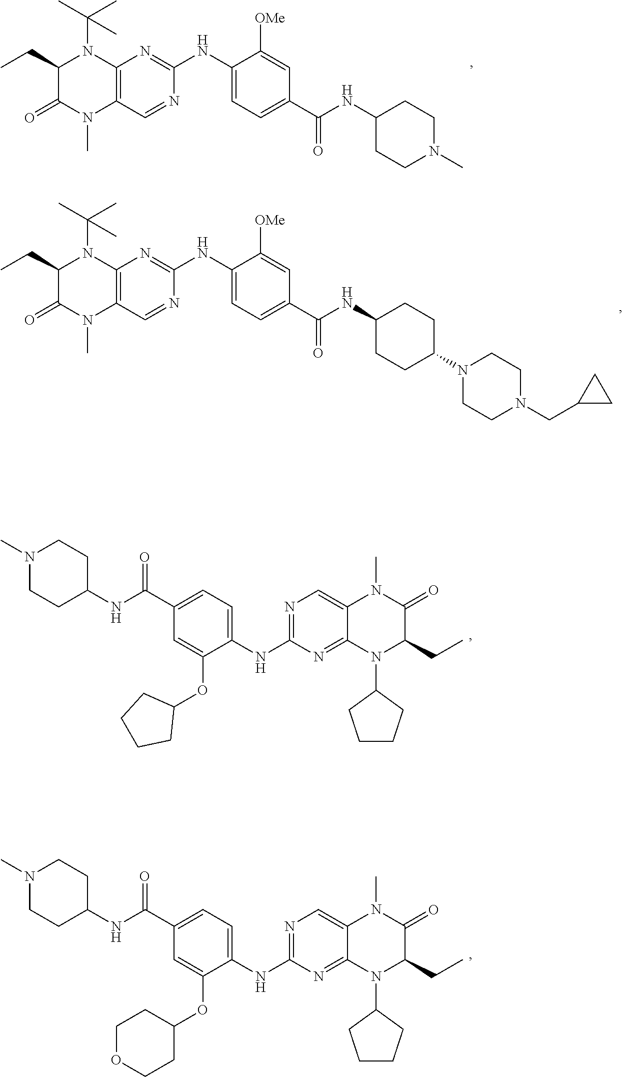

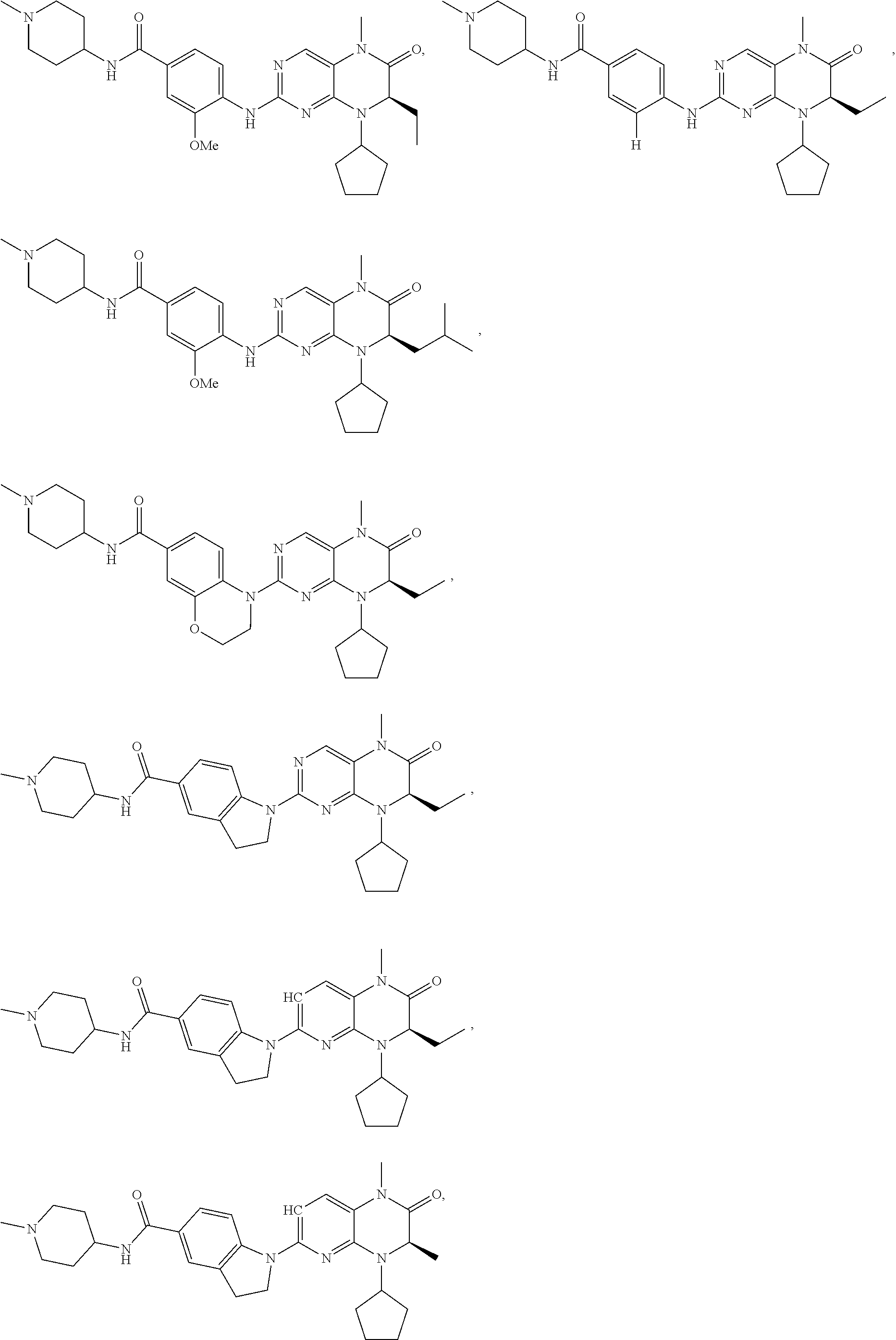

or a pharmaceutically acceptable salt, solvate, hydrate, polymorph, co-crystal, tautomer, stereoisomer, isotopically labeled derivative, or prodrug thereof.

### Compounds of Formula (IX)

In certain embodiments, the bromodomain inhibitor is an inhibitor disclosed in international PCT Publication No. WO 2015/117053, which is incorporated herein by reference.

In certain embodiments, the bromodomain inhibitor is of Formula (IX):

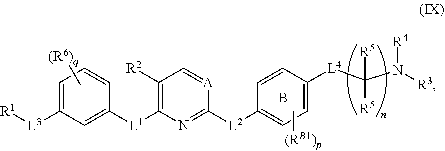

or pharmaceutically acceptable salt, solvate, hydrate, polymorph, co-crystal, tautomer, stereoisomer, isotopically labeled derivative, or prodrug thereof, wherein:
R¹ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group when attached to a nitrogen atom;
R² is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{D1}, —N(R^{D1})₂, —SR^{D1}, —CN, —SCN, —C(═NR^{D1})R^{D1}, —C(═NR^{D1})OR^{D1}, —C(═NR^{D1})N(R^{D1})₂, —C(═O)R^{D1}, —C(═O)OR^{D1}, —C(═O)N(R^{D1})₂, —NO₂, —NR^{D1}C(═O)R^{D1}, —NR^{D1}C(═O)OR^{D1}, —NR^{D1}C(═O)N(R^{D1})₂, —OC(═O)R^{D1}, —OC(═O)OR^{D1}, or —OC(—O)N(R^{D1})₂, wherein each instance of R^{D1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{D1} groups are joined to form a substituted or unsubstituted heterocyclic or substituted or unsubstituted heteroaryl ring;
R³ and R⁴ are each independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group; or R³ and R⁴ groups are joined to form an substituted or unsubstituted heterocyclic or substituted or unsubstituted heteroaryl ring;
each instance of R⁵ is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
each instance of R⁶ is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{B1a}, —N(R^{B1a})₂, —SR^{B1a}, —CN, —SCN, —C(═NR^{B1a})R^{B1a}, —C(═NR^{B1a})OR^{B1a}, —C(═NR^{B1a})N(R^{B1a})₂, —C(═O)R^{B1a}, —C(═O)OR^{B1a}, —C(═O)N(R^{B1a})₂, —NO₂, —NR^{B1a}C(═O)R^{B1a}, —NR^{B1a}C(═O)OR^{B1a}, —NR^{B1a}C(═O)N(R^{B1a})₂, —OC(═O)R^{B1a}, —OC(═O)OR^{B1a}, or —OC(═O)N(R^{B1a})₂;
q is 0, 1, 2, 3, or 4;
A is ═N— or ═C(R²)—;
each instance of R^{B1} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{B1a}, —N(R^{B1a})₂, —SR^{B1a}, —CN, —SCN, —C(═NR^{B1a})R^{B1a}, —C(═NR^{B1a})OR^{B1a}, —C(═NR^{B1a})N(R^{B1a})₂, —C(═O)R^{B1a}, —C(═O)OR^{B1a}, —C(═O)N(R^{B1a})₂, —NO₂, —NR^{B1a}C(═O)R^{B1a}, —NR^{B1a}C(═O)OR^{B1a}, —NR^{B1a}C(═O)N(R^{B1a})₂, —OC(═O)R^{B1a}, —OC(═O)OR^{B1a}, or —OC(═O)N(R^{B1a})₂;
each instance of R^{B1a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B1a} groups are joined to form a substituted or unsubstituted heterocyclic or substituted or unsubstituted heteroaryl ring;
p is 0 or an integer between 1 and 4, inclusive;
n is 0, 1, 2, 3, 4, 5, or 6;
L¹, L², and L⁴ are each independently a bond,

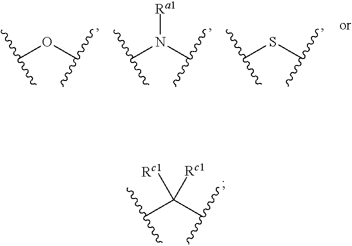

L³ is

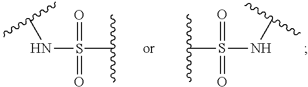 [SMILES: *NS(*)(=O)=O ; *NS(*)(=O)=O]

R^{a1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group; and
each instance of R^{c1} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{c1a}, —N(R^{c1a})₂, —SR^{c1a}, —CN, —C(═O)R^{c1a}, —C(═O)OR^{c1a}, —C(═O)N(R^{c1a})₂, —NR^{c1a}C(═O)R^{c1a}, —NR^{c1a}C(═O)OR^{c1a}, —NR^{c1a}C(═O)N(R^{c1a})₂, —OC(═O)R^{c1a}, or —OC(═O)N(R^{c1a})₂, wherein each instance of R^{c1a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{c1a} groups are joined to form a substituted or unsubstituted heterocyclic or substituted or unsubstituted heteroaryl ring.

In certain embodiments, the compound of Formula (IX) is of Formula (IX-A):

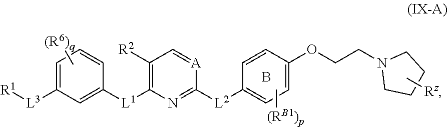

or a pharmaceutically acceptable salt, solvate, hydrate, polymorph, co-crystal, tautomer, stereoisomer, isotopically labeled derivative, or prodrug thereof, wherein:
R^{Z} is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted acyl, substituted or unsubstituted heteroaryl, —OR^{z1}, —N(R^{z1})₂, —SR^{z1}, —CN, —SCN, —C(═NR^{z1})R^{z1}, —C(═NR^{z1})OR^{z1}, —C(═NR^{z1})N(R^{z1})₂, —C(═O)R^{z1}, —C(═O)OR^{z1}, —C(═O)N(R_{z1})₂, —NO₂, —NR^{z1}C(═O)R^{z1}, —NR^{z1}C(═O)OR^{z1}, —NR^{z1}C(═O)N(R^{z1})₂, —OC(═O)R^{z1}, —OC(═O)OR^{z1}, or —OC(═O)N(R^{z1})₂, wherein each instance of R^{z1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{z1} groups are joined to form a substituted or unsubstituted heterocyclic or substituted or unsubstituted heteroaryl ring.

In certain embodiments, the compound of Formula (IX) is of Formula (IX-B):

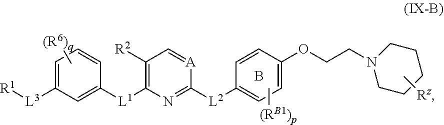

or a pharmaceutically acceptable salt, solvate, hydrate, polymorph, co-crystal, tautomer, stereoisomer, isotopically labeled derivative, or prodrug thereof, wherein:
R^{Z} is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{z1}, —N(R^{z1})₂, —SR^{z1}, —CN, —SCN, —C(═NR^{z1})R^{z1}, —C(═NR^{z1})OR^{z1}, —C(═NR^{z1})N(R^{a1})₂, —C(═O)R^{z1}, —C(═O)OR^{z1}, —C(═O)N(R^{z1})₂, —NO₂, —NR^{z1}C(═O)R^{z1}, —NR^{z1}C(═O)OR^{z1}, —NR^{z1}C(═O)N(R^{z1})₂, —OC(═O)R^{z1}, —OC(═O)OR^{z1}, or —OC(═O)N(R^{z1})₂, wherein each instance of R^{z1} is independently hydrogen, substituted or unsubstituted acyl, substituted or unsubstituted alkyl substituted or unsubstituted alkenyl, substituted or unsubstituted alkynyl, substituted or unsubstituted carbocyclyl, substituted or unsubstituted heterocyclyl, substituted or unsubstituted acyl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{z1} groups are joined to form a substituted or unsubstituted heterocyclic or substituted or unsubstituted heteroaryl ring.

In certain embodiments, the compound of Formula (IX) is of Formula (IX-C):

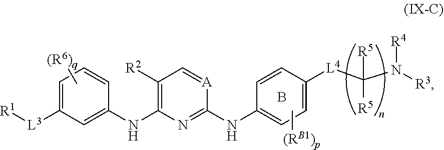

or pharmaceutically acceptable salt, solvate, hydrate, polymorph, co-crystal, tautomer, stereoisomer, isotopically labeled derivative, or prodrug thereof.

In certain embodiments, the compound of Formula (IX) is of Formula (IX-D):

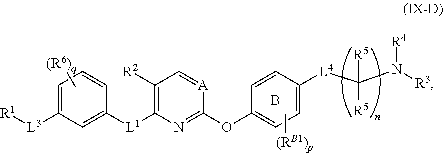

or pharmaceutically acceptable salt, solvate, hydrate, polymorph, co-crystal, tautomer, stereoisomer, isotopically labeled derivative, or prodrug thereof.

In certain embodiments, the compound of Formula (IX) is of Formula (IX-E):

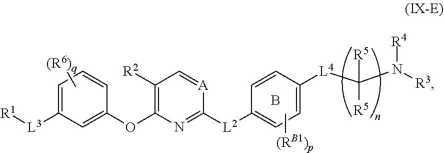

or pharmaceutically acceptable salt, solvate, hydrate, polymorph, co-crystal, tautomer, stereoisomer, isotopically labeled derivative, or prodrug thereof.

In certain embodiments, the compound of Formula (IX) is of Formula (IX-F):

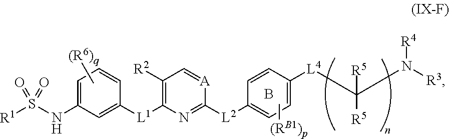

or pharmaceutically acceptable salt, solvate, hydrate, polymorph, co-crystal, tautomer, stereoisomer, isotopically labeled derivative, or prodrug thereof.

In certain embodiments, the compound of Formula (IX) is of Formula (IX-G):

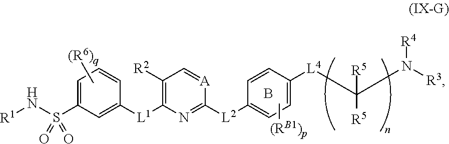

or pharmaceutically acceptable salt, solvate, hydrate, polymorph, co-crystal, tautomer, stereoisomer, isotopically labeled derivative, or prodrug thereof.

In certain embodiments, the bromodomain inhibitor is of the formula:

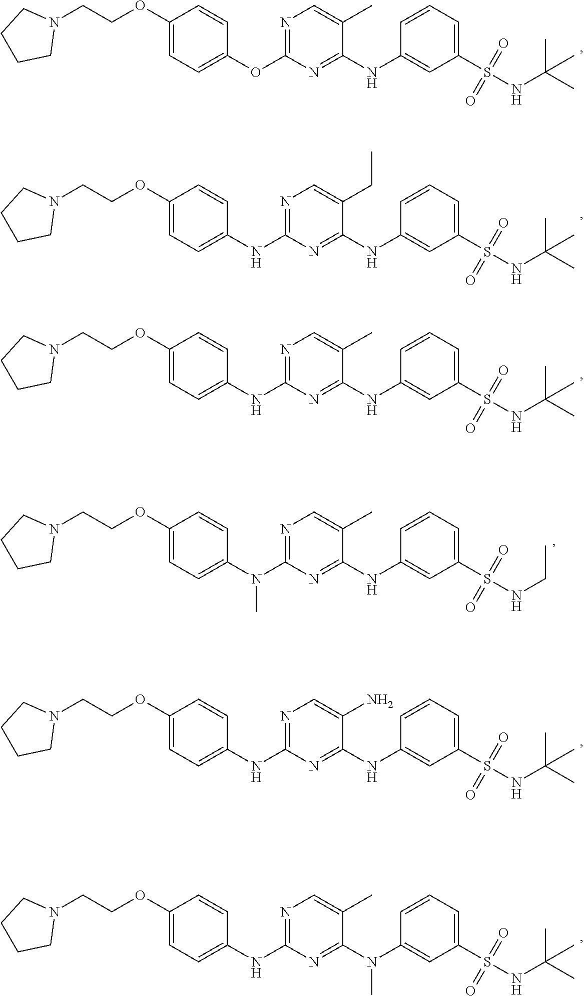

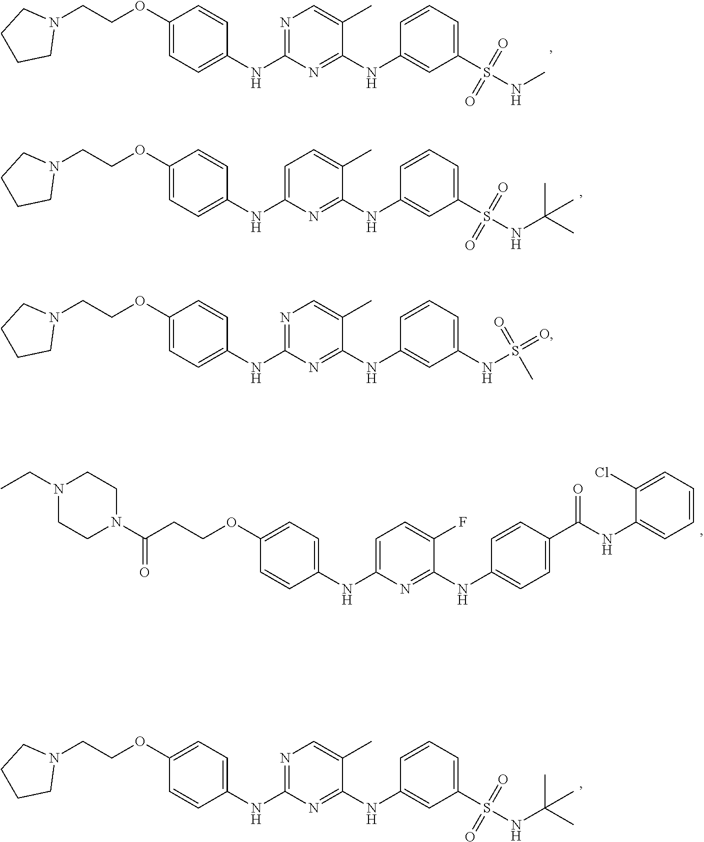

or a pharmaceutically acceptable salt, solvate, hydrate, polymorph, co-crystal, tautomer, stereoisomer, isotopically labeled derivative, or prodrug thereof.

### Compounds of Formula (X)

In certain embodiments, the bromodomain inhibitor is an inhibitor disclosed in WIPO Application No. PCT/US2015/44180, filed Aug. 7, 2015, which is incorporated herein by reference.

In certain embodiments, the bromodomain inhibitor is of Formula (X):

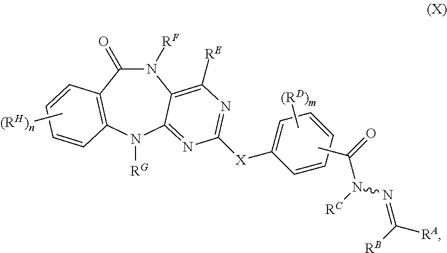

or a pharmaceutically acceptable salt, solvate, hydrate, polymorph, co-crystal, tautomer, stereoisomer, isotopically labeled derivative, or prodrug thereof, wherein:
R^{A} is hydrogen, substituted or unsubstituted acyl, substituted or unsubstituted alkyl, substituted or unsubstituted alkenyl, substituted or unsubstituted alkenyl, substituted or unsubstituted carbocyclyl, substituted or unsubstituted heterocyclyl, substituted or unsubstituted aryl, or substituted or unsubstituted heteroaryl;
R^{B} is hydrogen, substituted or unsubstituted acyl, substituted or unsubstituted substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R^{A} and R^{B} are joined to form a substituted or unsubstituted, carbocyclic ring, or a substituted or unsubstituted, heterocyclic ring;
R^{C} is hydrogen, substituted or unsubstituted C₁₋₆ alkyl, or a nitrogen protecting group;
each instance of R^{D} is independently halogen, substituted or unsubstituted substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —(C═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂;
each instance of R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{a} groups are joined to form a substituted or unsubstituted, heterocyclic ring, or a substituted or unsubstituted, heteroaryl ring;
m is 0, 1, 2, 3, or 4;
X is —O—, —S—, —N(R^{X1})—, or —C(R^{X2})₂—, wherein R^{X1} is hydrogen, substituted or unsubstituted C₁₋₆ alkyl, or a nitrogen protecting group, and wherein each instance of R^{X2} is independently hydrogen, halogen, or substituted or unsubstituted C₁₋₆ alkyl;
R^{E} is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, ——C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)OR^{a}, or —OC(═O)N(R^{a})₂;
R^{F} is hydrogen, substituted or unsubstituted C₁₋₆ alkyl, or a nitrogen protecting group;
R^{G} is hydrogen, substituted or unsubstituted alkyl, substituted or unsubstituted carbocyclyl, substituted or unsubstituted phenyl, or a nitrogen protecting group;
each instance of R^{H} is independently halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —N(R^{a})₂, —SR^{a}, —CN, —SCN, —C(═NR^{a})R^{a}, —C(═NR^{a})OR^{a}, —C(═NR^{a})N(R^{a})₂, —C(═O)R^{a}, —C(═O)OR^{a}, —C(═O)N(R^{a})₂, —NO₂, —NR^{a}C(═O)R^{a}, —NR^{a}C(═O)OR^{a}, —NR^{a}C(═O)N(R^{a})₂, —OC(═O)R^{a}, —OC(═O)OR^{a}, or —OC(═O)N(R^{a})₂; and
n is 0, 1, 2, 3, or 4.

In certain embodiments, the bromodomain inhibitor is of the formula:

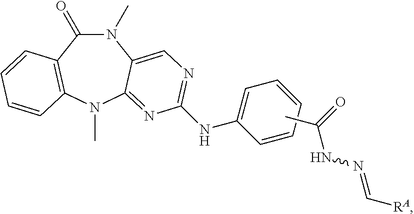

or a pharmaceutically acceptable salt, solvate, hydrate, polymorph, co-crystal, tautomer, stereoisomer, isotopically labeled derivative, or prodrug thereof, wherein R^{A} is selected from Table 1.

In certain embodiments, the bromodomain inhibitor is of the formula:

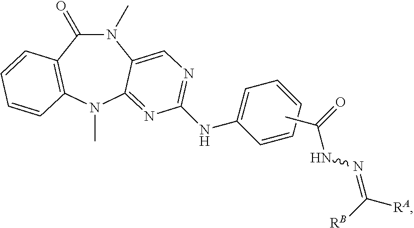

or a pharmaceutically acceptable salt, solvate, hydrate, polymorph, co-crystal, tautomer, stereoisomer, isotopically labeled derivative, or prodrug thereof, wherein R^{A} and R^{B} are independently selected from Table 2.

In certain embodiments, the bromodomain inhibitor is of the formula:

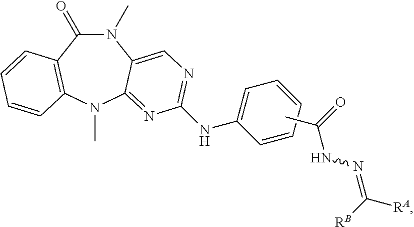

or a pharmaceutically acceptable salt, solvate, hydrate, polymorph, co-crystal, tautomer, stereoisomer, isotopically labeled derivative, or prodrug thereof, wherein

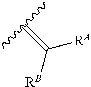

is selected from Table 3.

TABLE 1
R^{A}

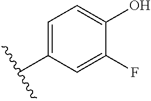 [SMILES: *c1ccc(O)c(F)c1]

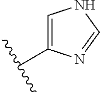 [SMILES: *c1c[nH]cn1]

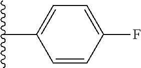 [SMILES: *c1ccc(F)cc1]

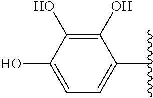 [SMILES: *c1ccc(O)c(O)c1O]

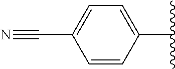 [SMILES: *c1ccc(C#N)cc1]

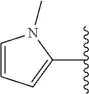 [SMILES: *c1cccn1C]

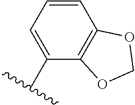 [SMILES: *c1cccc2c1OCO2]

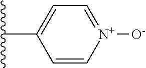 [SMILES: *c1cc[n+]([O-])cc1]

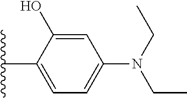 [SMILES: *c1ccc(N(CC)CC)cc1O]

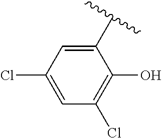 [SMILES: *c1cc(Cl)cc(Cl)c1O]

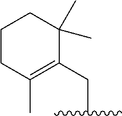 [SMILES: *CC1=C(C)CCCC1(C)C]

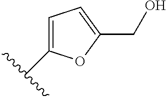 [SMILES: *c1ccc(CO)o1]

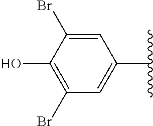 [SMILES: *c1cc(Br)c(O)c(Br)c1]

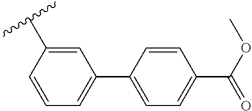 [SMILES: *c1cccc(-c2ccc(C(=O)OC)cc2)c1]

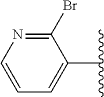 [SMILES: *c1cccnc1Br]

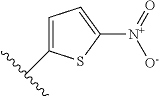 [SMILES: *c1ccc([N+](=O)[O-])s1]

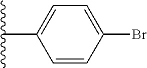 [SMILES: *c1ccc(Br)cc1]

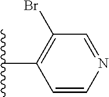 [SMILES: *c1ccncc1Br]

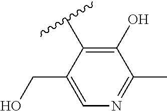 [SMILES: *c1c(CO)cnc(C)c1O]

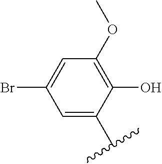 [SMILES: *c1cc(Br)cc(OC)c1O]

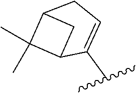 [SMILES: *C1=CCC2CC1C2(C)C]

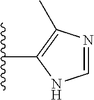 [SMILES: *c1[nH]cnc1C]

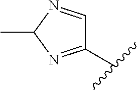 [SMILES: *C1=NC(C)N=C1]

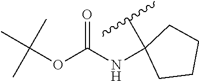 [SMILES: *C1(NC(=O)OC(C)(C)C)CCCC1]

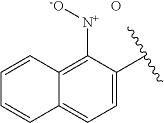

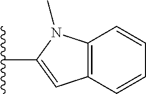 [SMILES: *c1cc2ccccc2n1C]

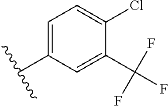 [SMILES: *c1ccc(Cl)c(C(F)(F)F)c1]

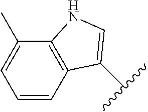 [SMILES: *c1c[nH]c2c(C)cccc12]

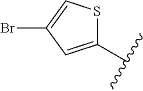 [SMILES: *c1cc(Br)cs1]

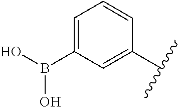 [SMILES: *c1cccc(B(O)O)c1]

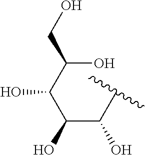 [SMILES: *[C@H](O)[C@@H](O)[C@H](O)[C@H](O)CO]

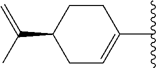 [SMILES: *C1=CC[C@@H](C(=C)C)CC1]

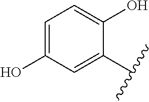 [SMILES: *c1cc(O)ccc1O]

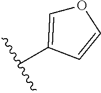 [SMILES: *c1ccoc1]

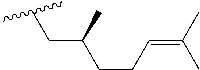 [SMILES: *C[C@@H](C)CCC=C(C)C]

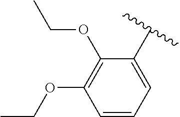 [SMILES: *c1cccc(OCC)c1OCC]

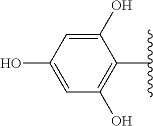 [SMILES: *c1c(O)cc(O)cc1O]

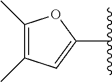 [SMILES: *c1cc(C)c(C)o1]

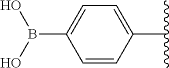 [SMILES: *c1ccc(B(O)O)cc1]

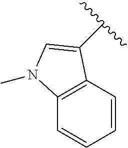 [SMILES: *c1cn(C)c2ccccc12]

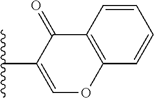 [SMILES: *c1coc2ccccc2c1=O]

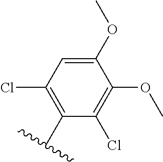 [SMILES: *c1c(Cl)cc(OC)c(OC)c1Cl]

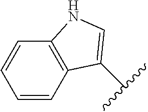 [SMILES: *c1c[nH]c2ccccc12]

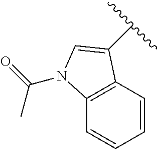 [SMILES: *c1cn(C(C)=O)c2ccccc12]

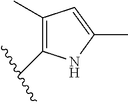

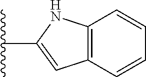 [SMILES: *c1cc2ccccc2[nH]1]

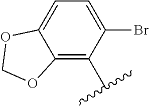 [SMILES: *c1c(Br)ccc2c1OCO2]

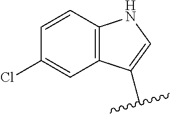 [SMILES: *c1c[nH]c2ccc(Cl)cc12]

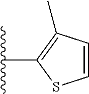 [SMILES: *c1sccc1C]

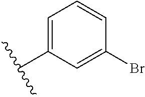 [SMILES: *c1cccc(Br)c1]

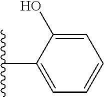 [SMILES: *c1ccccc1O]

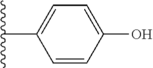 [SMILES: *c1ccc(O)cc1]

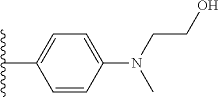 [SMILES: *c1ccc(N(C)CCO)cc1]

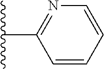 [SMILES: *c1ccccn1]

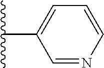 [SMILES: *c1cccnc1]

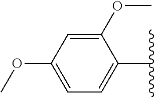

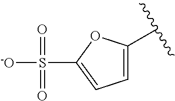

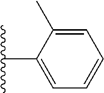 [SMILES: *c1ccccc1C]

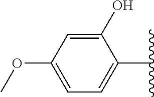 [SMILES: *c1ccc(OC)cc1O]

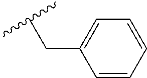 [SMILES: *Cc1ccccc1]

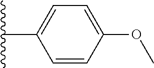 [SMILES: *c1ccc(OC)cc1]

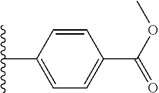 [SMILES: *c1ccc(C(=O)OC)cc1]

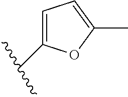 [SMILES: *c1ccc(C)o1]

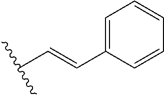 [SMILES: */C=C/c1ccccc1]

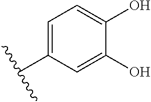 [SMILES: *c1ccc(O)c(O)c1]

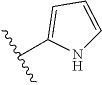 [SMILES: *c1ccc[nH]1]

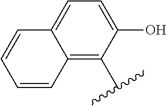 [SMILES: *c1c(O)ccc2ccccc12]

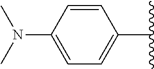 [SMILES: *c1ccc(N(C)C)cc1]

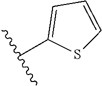 [SMILES: *c1cccs1]

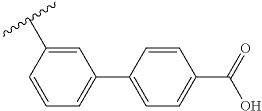 [SMILES: *c1cccc(-c2ccc(C(=O)O)cc2)c1]

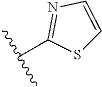 [SMILES: *c1nccs1]

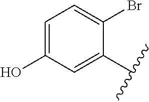 [SMILES: *c1cc(O)ccc1Br]

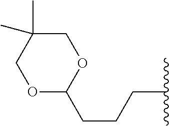 [SMILES: *CCCC1OCC(C)(C)CO1]

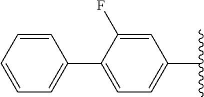 [SMILES: *c1ccc(-c2ccccc2)c(F)c1]

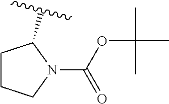 [SMILES: *[C@H]1CCCN1C(=O)OC(C)(C)C]

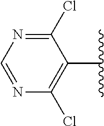 [SMILES: *c1c(Cl)ncnc1Cl]

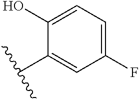 [SMILES: *c1cc(F)ccc1O]

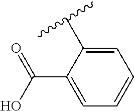 [SMILES: *c1ccccc1C(=O)O]

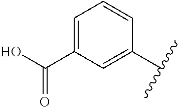 [SMILES: *c1cccc(C(=O)O)c1]

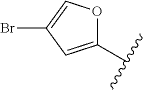 [SMILES: *c1cc(Br)co1]

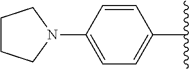 [SMILES: *c1ccc(N2CCCC2)cc1]

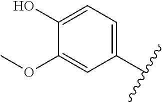 [SMILES: *c1ccc(O)c(OC)c1]

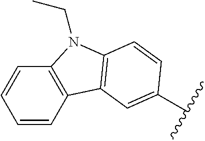 [SMILES: *c1ccc2c(c1)c1ccccc1n2CC]

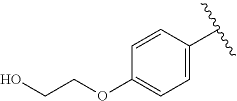 [SMILES: *c1ccc(OCCO)cc1]

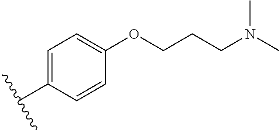 [SMILES: *c1ccc(OCCCN(C)C)cc1]

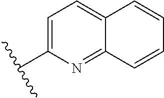 [SMILES: *c1ccc2ccccc2n1]

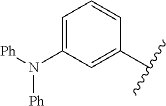 [SMILES: *c1cccc(N(c2ccccc2)c2ccccc2)c1]

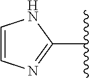 [SMILES: *c1ncc[nH]1]

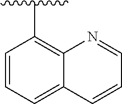 [SMILES: *c1cccc2cccnc12]

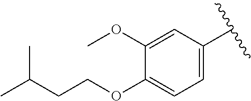 [SMILES: *c1ccc(OCCC(C)C)c(OC)c1]

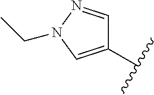 [SMILES: *c1cnn(CC)c1]

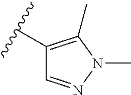 [SMILES: *c1cnn(C)c1C]

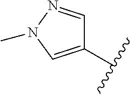 [SMILES: *c1cnn(C)c1]

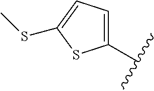 [SMILES: *c1ccc(SC)s1]

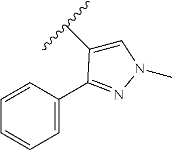 [SMILES: *c1cn(C)nc1-c1ccccc1]

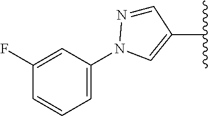 [SMILES: *c1cnn(-c2cccc(F)c2)c1]

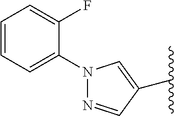 [SMILES: *c1cnn(-c2ccccc2F)c1]

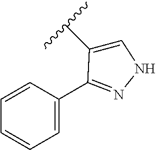 [SMILES: *c1c[nH]nc1-c1ccccc1]

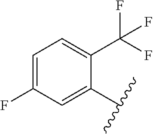 [SMILES: *c1cc(F)ccc1C(F)(F)F]

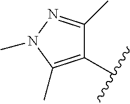 [SMILES: *c1c(C)nn(C)c1C]

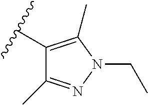 [SMILES: *c1c(C)nn(CC)c1C]

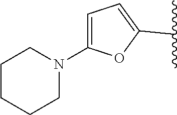 [SMILES: *c1ccc(N2CCCCC2)o1]

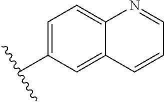 [SMILES: *c1ccc2ncccc2c1]

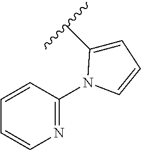 [SMILES: *c1cccn1-c1ccccn1]

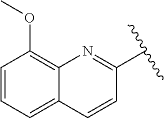 [SMILES: *c1ccc2cccc(OC)c2n1]

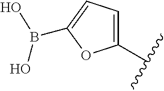 [SMILES: *c1ccc(B(O)O)o1]

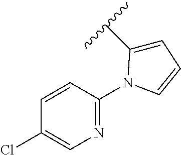 [SMILES: *c1cccn1-c1ccc(Cl)cn1]

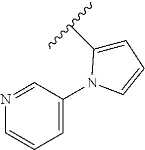 [SMILES: *c1cccn1-c1cccnc1]

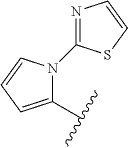 [SMILES: *c1cccn1-c1nccs1]

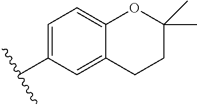 [SMILES: *c1ccc2c(c1)CCC(C)(C)O2]

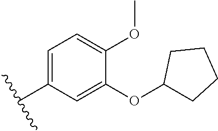 [SMILES: *c1ccc(OC)c(OC2CCCC2)c1]

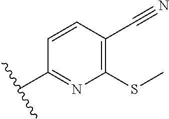 [SMILES: *c1ccc(C#N)c(SC)n1]

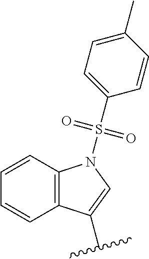 [SMILES: *c1cn(S(=O)(=O)c2ccc(C)cc2)c2ccccc12]

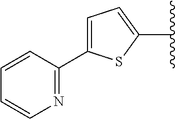 [SMILES: *c1ccc(-c2ccccn2)s1]

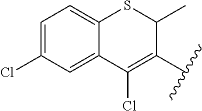 [SMILES: *C1=C(Cl)c2cc(Cl)ccc2SC1C]

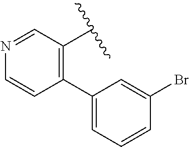 [SMILES: *c1cnccc1-c1cccc(Br)c1]

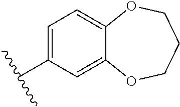 [SMILES: *c1ccc2c(c1)OCCCO2]

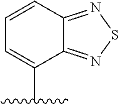 [SMILES: *c1cccc2nsnc12]

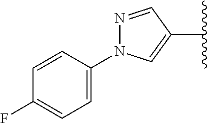 [SMILES: *c1cnn(-c2ccc(F)cc2)c1]

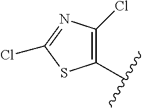 [SMILES: *c1sc(Cl)nc1Cl]

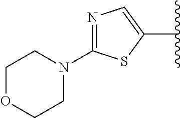 [SMILES: *c1cnc(N2CCOCC2)s1]

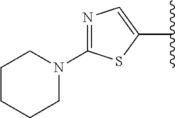 [SMILES: *c1cnc(N2CCCCC2)s1]

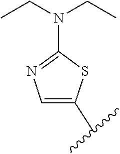 [SMILES: *c1cnc(N(CC)CC)s1]

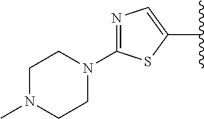 [SMILES: *c1cnc(N2CCN(C)CC2)s1]

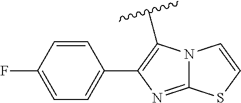 [SMILES: *c1c(-c2ccc(F)cc2)nc2sccn12]

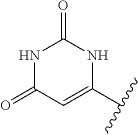 [SMILES: *c1cc(=O)[nH]c(=O)[nH]1]

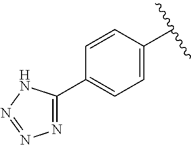 [SMILES: *c1ccc(-c2nnn[nH]2)cc1]

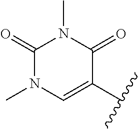 [SMILES: *c1cn(C)c(=O)n(C)c1=O]

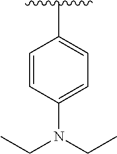 [SMILES: *c1ccc(N(CC)CC)cc1]

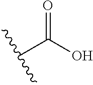 [SMILES: *C(=O)O]

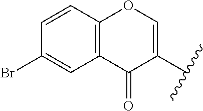 [SMILES: *c1coc2ccc(Br)cc2c1=O]

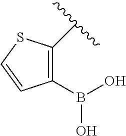 [SMILES: *c1sccc1B(O)O]

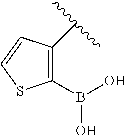 [SMILES: *c1ccsc1B(O)O]

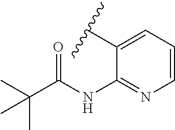 [SMILES: *c1cccnc1NC(=O)C(C)(C)C]

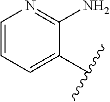 [SMILES: *c1cccnc1N]

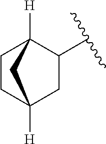 [SMILES: *C1C[C@@H]2CC[C@H]1C2]

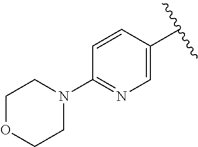 [SMILES: *c1ccc(N2CCOCC2)nc1]

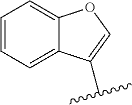 [SMILES: *c1coc2ccccc12]

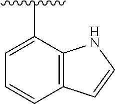 [SMILES: *c1cccc2cc[nH]c12]

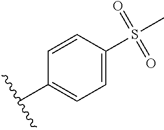 [SMILES: *c1ccc(S(C)(=O)=O)cc1]

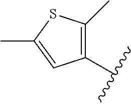 [SMILES: *c1cc(C)sc1C]

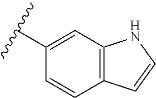 [SMILES: *c1ccc2cc[nH]c2c1]

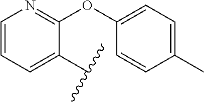 [SMILES: *c1cccnc1Oc1ccc(C)cc1]

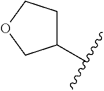 [SMILES: *C1CCOC1]

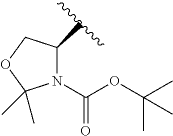 [SMILES: *[C@@H]1COC(C)(C)N1C(=O)OC(C)(C)C]

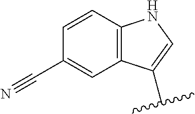 [SMILES: *c1c[nH]c2ccc(C#N)cc12]

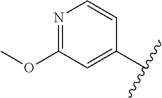 [SMILES: *c1ccnc(OC)c1]

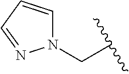 [SMILES: *Cn1cccn1]

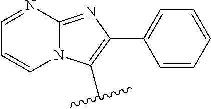 [SMILES: *c1c(-c2ccccc2)nc2ncccn12]

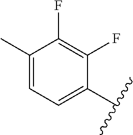 [SMILES: *c1ccc(C)c(F)c1F]

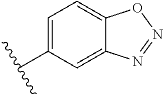 [SMILES: *c1ccc2onnc2c1]

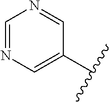 [SMILES: *c1cncnc1]

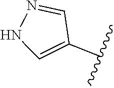 [SMILES: *c1cn[nH]c1]

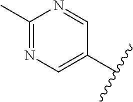 [SMILES: *c1cnc(C)nc1]

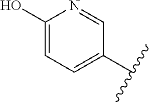 [SMILES: *c1ccc(O)nc1]

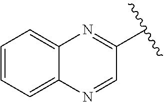 [SMILES: *c1cnc2ccccc2n1]

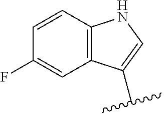 [SMILES: *c1c[nH]c2ccc(F)cc12]

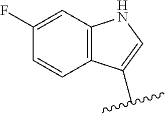 [SMILES: *c1c[nH]c2cc(F)ccc12]

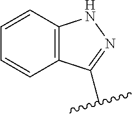 [SMILES: *c1n[nH]c2ccccc12]

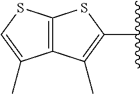 [SMILES: *c1sc2scc(C)c2c1C]

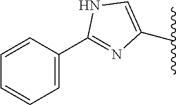 [SMILES: *c1c[nH]c(-c2ccccc2)n1]

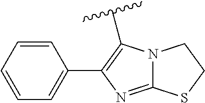 [SMILES: *c1c(-c2ccccc2)nc2n1CCS2]

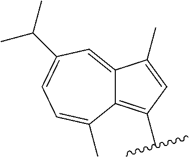 [SMILES: *c1cc(C)c2cc(C(C)C)ccc(C)c1-2]

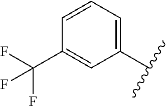 [SMILES: *c1cccc(C(F)(F)F)c1]

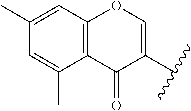 [SMILES: *c1coc2cc(C)cc(C)c2c1=O]

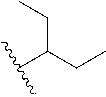 [SMILES: *C(CC)CC]

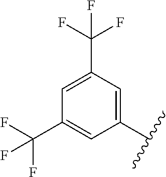 [SMILES: *c1cc(C(F)(F)F)cc(C(F)(F)F)c1]

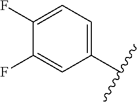 [SMILES: *c1ccc(F)c(F)c1]

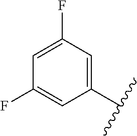 [SMILES: *c1cc(F)cc(F)c1]

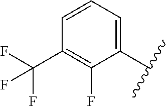 [SMILES: *c1cccc(C(F)(F)F)c1F]

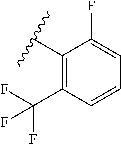 [SMILES: *c1c(F)cccc1C(F)(F)F]

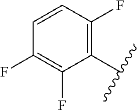 [SMILES: *c1c(F)ccc(F)c1F]

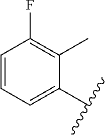 [SMILES: *c1cccc(F)c1C]

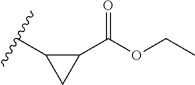 [SMILES: *C1CC1C(=O)OCC]

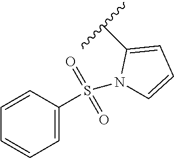 [SMILES: *c1cccn1S(=O)(=O)c1ccccc1]

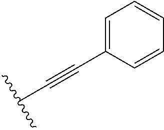 [SMILES: *C#Cc1ccccc1]

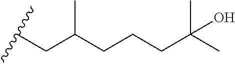 [SMILES: *CC(C)CCCC(C)(C)O]

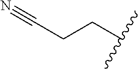 [SMILES: *CCC#N]

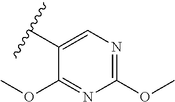

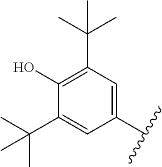 [SMILES: *c1cc(C(C)(C)C)c(O)c(C(C)(C)C)c1]

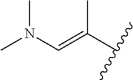 [SMILES: */C(C)=C/N(C)C]

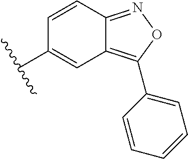 [SMILES: *c1ccc2noc(-c3ccccc3)c2c1]

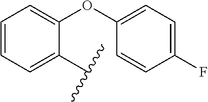 [SMILES: *c1ccccc1Oc1ccc(F)cc1]

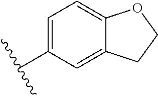 [SMILES: *c1ccc2c(c1)CCO2]

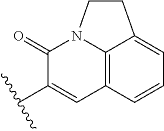 [SMILES: *c1cc2cccc3c2n(c1=O)CC3]

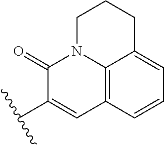 [SMILES: *c1cc2cccc3c2n(c1=O)CCC3]

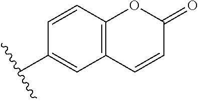 [SMILES: *c1ccc2oc(=O)ccc2c1]

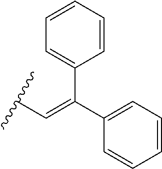 [SMILES: *C=C(c1ccccc1)c1ccccc1]

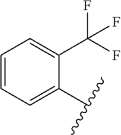 [SMILES: *c1ccccc1C(F)(F)F]

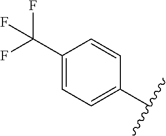 [SMILES: *c1ccc(C(F)(F)F)cc1]

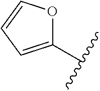 [SMILES: *c1ccco1]

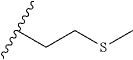 [SMILES: *CCSC]

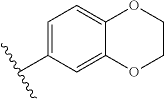 [SMILES: *c1ccc2c(c1)OCCO2]

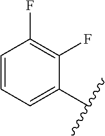 [SMILES: *c1cccc(F)c1F]

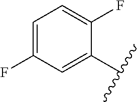 [SMILES: *c1cc(F)ccc1F]

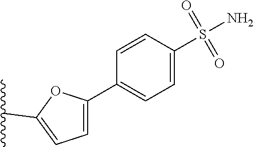 [SMILES: *c1ccc(-c2ccc(S(N)(=O)=O)cc2)o1]

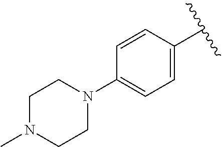 [SMILES: *c1ccc(N2CCN(C)CC2)cc1]

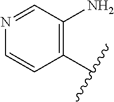 [SMILES: *c1ccncc1N]

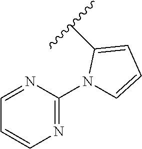 [SMILES: *c1cccn1-c1ncccn1]

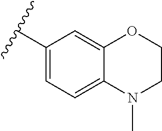 [SMILES: *c1ccc2c(c1)OCCN2C]

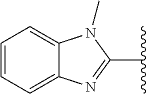 [SMILES: *c1nc2ccccc2n1C]

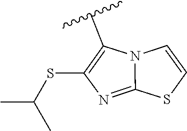 [SMILES: *c1c(SC(C)C)nc2sccn12]

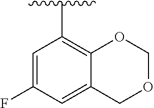 [SMILES: *c1cc(F)cc2c1OCOC2]

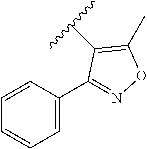 [SMILES: *c1c(-c2ccccc2)noc1C]

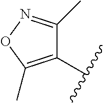 [SMILES: *c1c(C)noc1C]

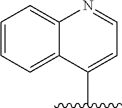 [SMILES: *c1ccnc2ccccc12]

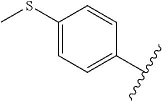 [SMILES: *c1ccc(SC)cc1]

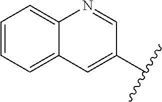 [SMILES: *c1cnc2ccccc2c1]

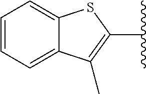 [SMILES: *c1sc2ccccc2c1C]

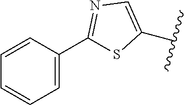 [SMILES: *c1cnc(-c2ccccc2)s1]

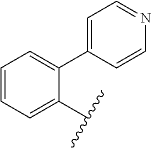 [SMILES: *c1ccccc1-c1ccncc1]

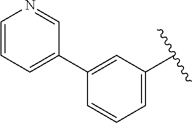 [SMILES: *c1cccc(-c2cccnc2)c1]

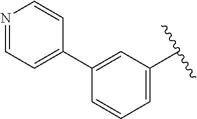 [SMILES: *c1cccc(-c2ccncc2)c1]

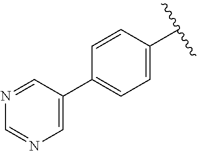 [SMILES: *c1ccc(-c2cncnc2)cc1]

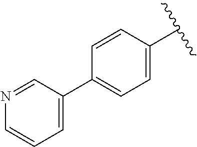 [SMILES: *c1ccc(-c2cccnc2)cc1]

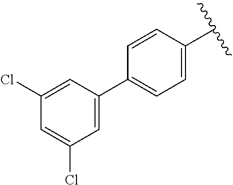 [SMILES: *c1ccc(-c2cc(Cl)cc(Cl)c2)cc1]

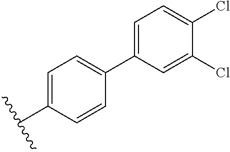 [SMILES: *c1ccc(-c2ccc(Cl)c(Cl)c2)cc1]

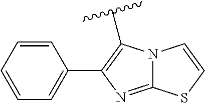 [SMILES: *c1c(-c2ccccc2)nc2sccn12]

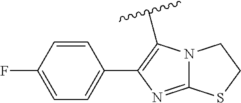 [SMILES: *c1c(-c2ccc(F)cc2)nc2n1CCS2]

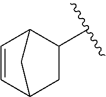 [SMILES: *C1CC2C=CC1C2]

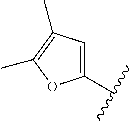 [SMILES: *c1cc(C)c(C)o1]

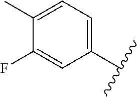 [SMILES: *c1ccc(C)c(F)c1]

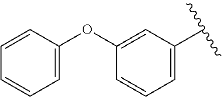 [SMILES: *c1cccc(Oc2ccccc2)c1]

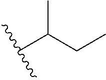 [SMILES: *C(C)CC]

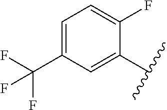 [SMILES: *c1cc(C(F)(F)F)ccc1F]

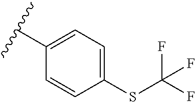 [SMILES: *c1ccc(SC(F)(F)F)cc1]

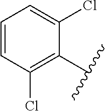 [SMILES: *c1c(Cl)cccc1Cl]

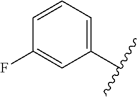 [SMILES: *c1cccc(F)c1]

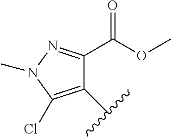

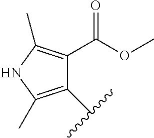 [SMILES: *c1c(C)[nH]c(C)c1C(=O)OC]

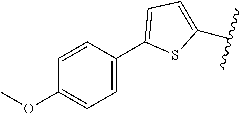 [SMILES: *c1ccc(-c2ccc(OC)cc2)s1]

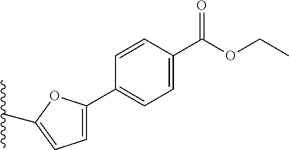 [SMILES: *c1ccc(-c2ccc(C(=O)OCC)cc2)o1]

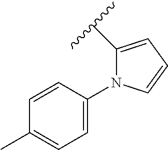 [SMILES: *c1cccn1-c1ccc(C)cc1]

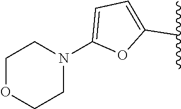 [SMILES: *c1ccc(N2CCOCC2)o1]

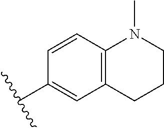 [SMILES: *c1ccc2c(c1)CCCN2C]

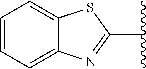 [SMILES: *c1nc2ccccc2s1]

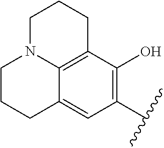 [SMILES: *c1cc2c3c(c1O)CCCN3CCC2]

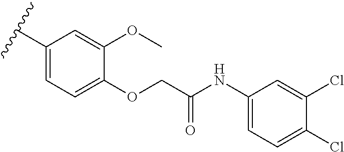 [SMILES: *c1ccc(OCC(=O)Nc2ccc(Cl)c(Cl)c2)c(OC)c1]

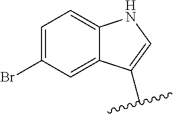 [SMILES: *c1c[nH]c2ccc(Br)cc12]

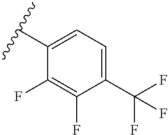 [SMILES: *c1ccc(C(F)(F)F)c(F)c1F]

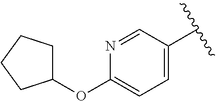 [SMILES: *c1ccc(OC2CCCC2)nc1]

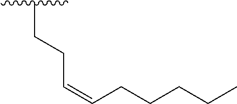 [SMILES: *CC/C=C\CCCCC]

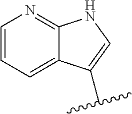 [SMILES: *c1c[nH]c2ncccc12]

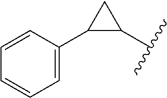 [SMILES: *C1CC1c1ccccc1]

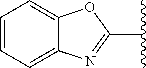 [SMILES: *c1nc2ccccc2o1]

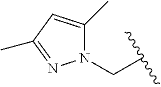 [SMILES: *Cn1nc(C)cc1C]

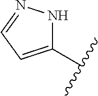 [SMILES: *c1ccn[nH]1]

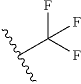 [SMILES: *C(F)(F)F]

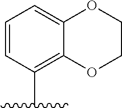 [SMILES: *c1cccc2c1OCCO2]

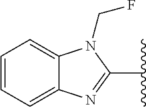 [SMILES: *c1nc2ccccc2n1CF]

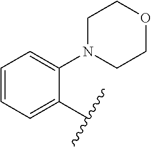 [SMILES: *c1ccccc1N1CCOCC1]

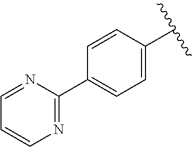 [SMILES: *c1ccc(-c2ncccn2)cc1]

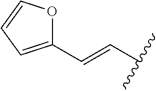 [SMILES: */C=C/c1ccco1]

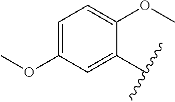

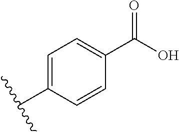 [SMILES: *c1ccc(C(=O)O)cc1]

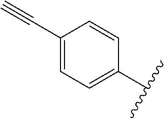 [SMILES: *c1ccc(C#C)cc1]

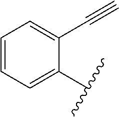 [SMILES: *c1ccccc1C#C]

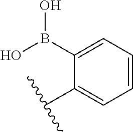 [SMILES: *c1ccccc1B(O)O]

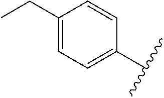 [SMILES: *c1ccc(CC)cc1]

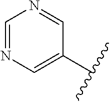 [SMILES: *c1cncnc1]

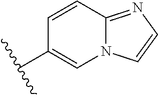 [SMILES: *c1ccc2nccn2c1]

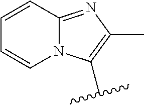 [SMILES: *c1c(C)nc2ccccn12]

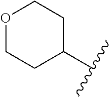 [SMILES: *C1CCOCC1]

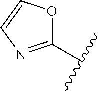 [SMILES: *c1ncco1]

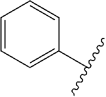 [SMILES: *c1ccccc1]

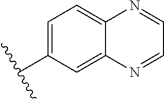 [SMILES: *c1ccc2nccnc2c1]

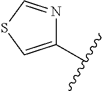 [SMILES: *c1cscn1]

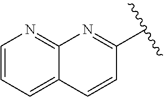 [SMILES: *c1ccc2cccnc2n1]

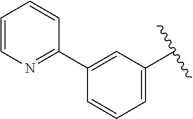 [SMILES: *c1cccc(-c2ccccn2)c1]

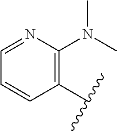 [SMILES: *c1cccnc1N(C)C]

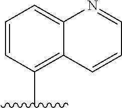 [SMILES: *c1cccc2ncccc12]

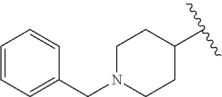 [SMILES: *C1CCN(Cc2ccccc2)CC1]

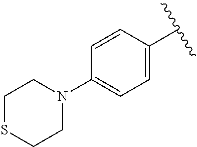 [SMILES: *c1ccc(N2CCSCC2)cc1]

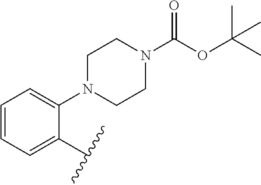 [SMILES: *c1ccccc1N1CCN(C(=O)OC(C)(C)C)CC1]

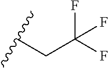 [SMILES: *CC(F)(F)F]

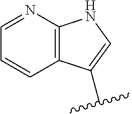 [SMILES: *c1c[nH]c2ncccc12]

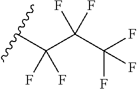 [SMILES: *C(F)(F)C(F)(F)C(F)(F)F]

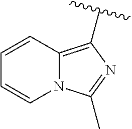 [SMILES: *c1nc(C)n2ccccc12]

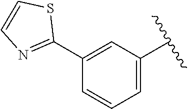 [SMILES: *c1cccc(-c2nccs2)c1]

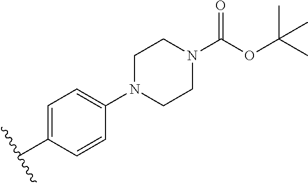 [SMILES: *c1ccc(N2CCN(C(=O)OC(C)(C)C)CC2)cc1]

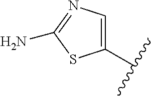 [SMILES: *c1cnc(N)s1]

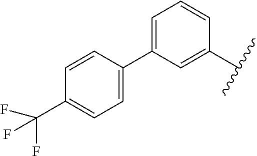 [SMILES: *c1cccc(-c2ccc(C(F)(F)F)cc2)c1]

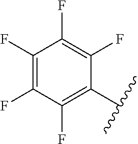 [SMILES: *c1c(F)c(F)c(F)c(F)c1F]

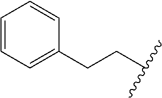 [SMILES: *CCc1ccccc1]

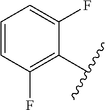 [SMILES: *c1c(F)cccc1F]

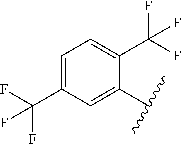 [SMILES: *c1cc(C(F)(F)F)ccc1C(F)(F)F]

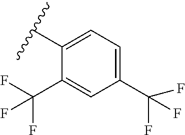 [SMILES: *c1ccc(C(F)(F)F)cc1C(F)(F)F]

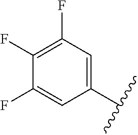 [SMILES: *c1cc(F)c(F)c(F)c1]

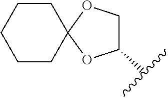 [SMILES: *[C@H]1COC2(CCCCC2)O1]

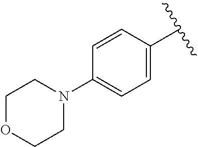 [SMILES: *c1ccc(N2CCOCC2)cc1]

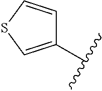 [SMILES: *c1ccsc1]

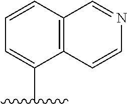 [SMILES: *c1cccc2cnccc12]

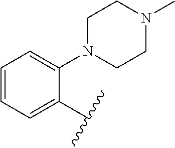 [SMILES: *c1ccccc1N1CCN(C)CC1]

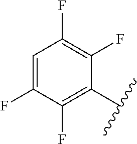 [SMILES: *c1c(F)c(F)cc(F)c1F]

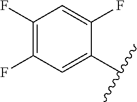 [SMILES: *c1cc(F)c(F)cc1F]

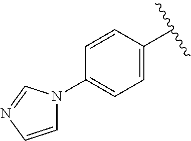 [SMILES: *c1ccc(-n2ccnc2)cc1]

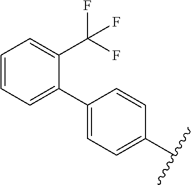 [SMILES: *c1ccc(-c2ccccc2C(F)(F)F)cc1]

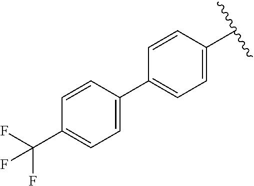 [SMILES: *c1ccc(-c2ccc(C(F)(F)F)cc2)cc1]

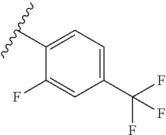 [SMILES: *c1ccc(C(F)(F)F)cc1F]

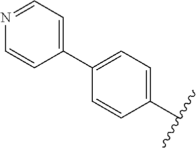 [SMILES: *c1ccc(-c2ccncc2)cc1]

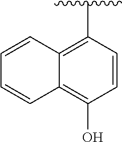 [SMILES: *c1ccc(O)c2ccccc12]

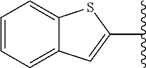 [SMILES: *c1cc2ccccc2s1]

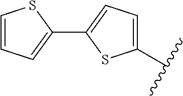 [SMILES: *c1ccc(-c2cccs2)s1]

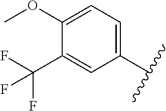 [SMILES: *c1ccc(OC)c(C(F)(F)F)c1]

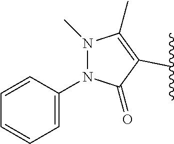 [SMILES: *c1c(C)n(C)n(-c2ccccc2)c1=O]

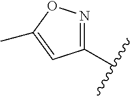 [SMILES: *c1cc(C)on1]

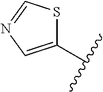 [SMILES: *c1cncs1]

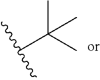 [SMILES: *C(C)(C)C]

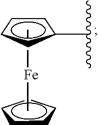

TABLE 2
R^{A} or R^{B}

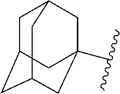 [SMILES: *C12CC3CC(CC(C3)C1)C2]

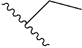 [SMILES: *CC]

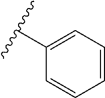 [SMILES: *c1ccccc1]

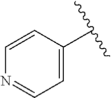 [SMILES: *c1ccncc1]

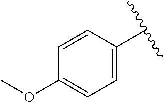 [SMILES: *c1ccc(OC)cc1]

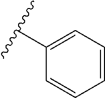 [SMILES: *c1ccccc1]

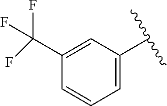 [SMILES: *c1cccc(C(F)(F)F)c1]

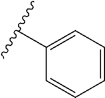 [SMILES: *c1ccccc1]

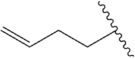 [SMILES: *CCC=C]

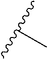

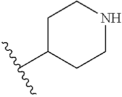 [SMILES: *C1CCNCC1]

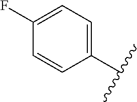 [SMILES: *c1ccc(F)cc1]

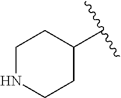 [SMILES: *C1CCNCC1]

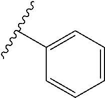 [SMILES: *c1ccccc1]

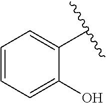 [SMILES: *c1ccccc1O]

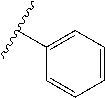 [SMILES: *c1ccccc1]

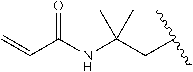 [SMILES: *CC(C)(C)NC(=O)C=C]

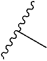

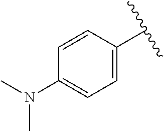 [SMILES: *c1ccc(N(C)C)cc1]

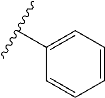 [SMILES: *c1ccccc1]

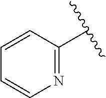 [SMILES: *c1ccccn1]

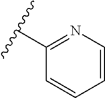 [SMILES: *c1ccccn1]

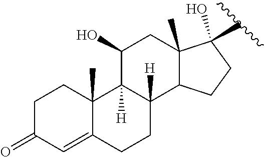

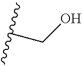 [SMILES: *CO]

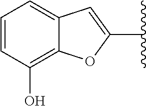 [SMILES: *c1cc2cccc(O)c2o1]

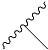

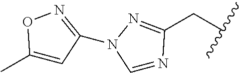 [SMILES: *Cc1ncn(-c2cc(C)on2)n1]

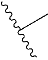

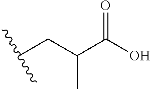 [SMILES: *CC(C)C(=O)O]

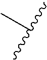

TABLE 3

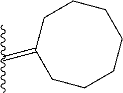 [SMILES: *=C1CCCCCCC1]

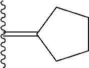 [SMILES: *=C1CCCC1]

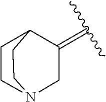 [SMILES: *=C1CN2CCC1CC2]

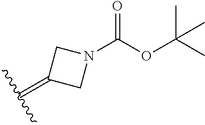 [SMILES: *=C1CN(C(=O)OC(C)(C)C)C1]

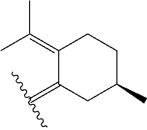 [SMILES: *=C1C[C@H](C)CCC1=C(C)C]

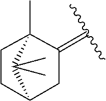 [SMILES: *=C1C[C@H]2CC[C@]1(C)C2(C)C]

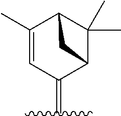

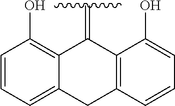 [SMILES: *=C1c2c(O)cccc2Cc2cccc(O)c21]

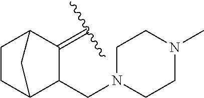 [SMILES: *=C1C2CCC(C2)C1CN1CCN(C)CC1]

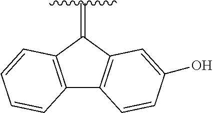 [SMILES: *=C1c2ccccc2-c2ccc(O)cc21]

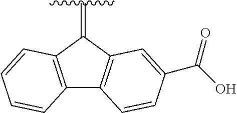 [SMILES: *=C1c2ccccc2-c2ccc(C(=O)O)cc21]

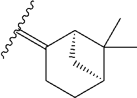 [SMILES: *=C1CC[C@H]2C[C@@H]1C2(C)C]

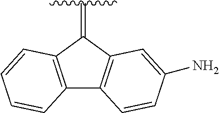 [SMILES: *=C1c2ccccc2-c2ccc(N)cc21]

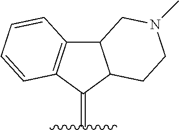 [SMILES: *=C1c2ccccc2C2CN(C)CCC12]

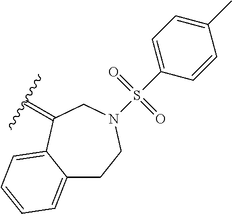 [SMILES: *=C1CN(S(=O)(=O)c2ccc(C)cc2)CCc2ccccc21]

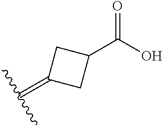 [SMILES: *=C1CC(C(=O)O)C1]

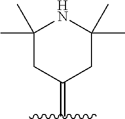

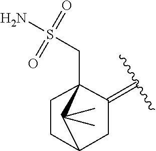 [SMILES: *=C1CC2CC[C@@]1(CS(N)(=O)=O)C2(C)C]

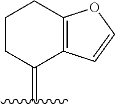 [SMILES: *=C1CCCc2occc21]

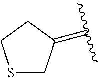 [SMILES: *=C1CCSC1]

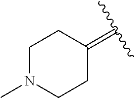 [SMILES: *=C1CCN(C)CC1]

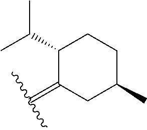 [SMILES: *=C1C[C@H](C)CC[C@H]1C(C)C]

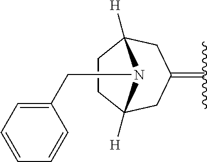 [SMILES: *=C1C[C@H]2CC[C@@H](C1)N2Cc1ccccc1]

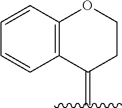 [SMILES: *=C1CCOc2ccccc21]

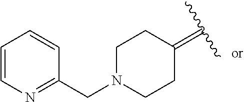 [SMILES: *=C1CCN(Cc2ccccn2)CC1]

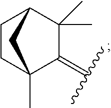 [SMILES: *=C1C(C)(C)[C@H]2CC[C@]1(C)C2]

### Compounds of Formula (XI)

In certain embodiments, the bromodomain inhibitor is an inhibitor disclosed in WIPO Application No. PCT/US2015/44303, filed Aug. 7, 2015, which is incorporated herein by reference.

In certain embodiments, the bromodomain inhibitor is of Formula (XI):

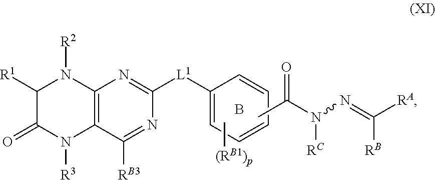

or pharmaceutically acceptable salt, solvate, hydrate, polymorph, co-crystal, tautomer, stereoisomer, isotopically labeled derivative, or prodrug thereof, wherein:
R^{A} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R^{B} is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
or R^{A} and R^{B} are joined to form a substituted or unsubstituted, carbocyclic ring, or a substituted or unsubstituted, heterocyclic ring;
R^{C} is hydrogen, substituted or unsubstituted C₁₋₆ alkyl, or a nitrogen protecting group;
R¹ is hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl;
R² and R³ are each independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —C(═O)R^{D1}, —C(═O)OR^{D1}, —C(═O)N(R^{D1})₂, or a nitrogen protecting group, wherein each instance of R^{D1} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{D1} groups are joined to form a substituted or unsubstituted heterocyclic or substituted or unsubstituted heteroaryl ring, or a nitrogen protecting group when attached to a nitrogen atom;
each instance of R^{B1} is independently hydrogen, halogen, substituted or unsubstituted alkyl, substituted or unsubstituted alkenyl, substituted or unsubstituted substituted or unsubstituted carbocyclyl, substituted or unsubstituted heterocyclyl, substituted or unsubstituted aryl, substituted or unsubstituted heteroaryl, —OR^{B1a}, —N(R^{B1a})₂, —SR^{B1a}, —CN, —SCN, —C(═NR^{B1a})R^{B1a}, —C(═NR^{B1a})OR^{B1a}, —C(═NR^{B1a})N(R^{B1a})₂, —C(═O)R^{B1a}, —C(═O)OR^{B1a}, —C(═O)N(R^{B1a})₂, —NO₂, —NR^{B1a}C(═O)R^{B1a}, —NR^{B1a}C(═O)OR^{B1a}, —NR^{B1a}C(═O)N(R^{B1a})₂, —OC(═O)R^{B1a}, —OC(═O)OR^{B1a}, or —OC(═O)N(R^{B1a})₂, wherein each instance of R^{B1a} is independently hydrogen, substituted or unsubstituted acyl, substituted or unsubstituted substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom or about two R^{B1a} groups are joined to form a substituted or unsubstituted heterocyclic or substituted or unsubstituted heteroaryl ring;
each instance of R^{B3} is independently hydrogen, halogen, substituted or unsubstituted alkyl, substituted or unsubstituted alkenyl, substituted or unsubstituted substituted or unsubstituted carbocyclyl, substituted or unsubstituted heterocyclyl, substituted or unsubstituted aryl, substituted or unsubstituted heteroaryl, —OR^{B3a}, —N(R^{B3a})₂, —SR^{B3a}, —CN, —SCN, —C(═NR^{B3a})R^{B3a}, —C(═NR^{B3a})OR^{B3a}, —C(═NR^{B3a})N(R^{B3a})₂, —C(═O)R^{B3a}, —C(═O)OR^{B3a}, —C(═O)N(R^{B3a})₂, —NO₂, —NR^{B3a}C(═O)R^{B3a}, —NR^{B3a}C(═O)OR^{B3a}, —NR^{B3a}C(═O)N(R^{B3a})₂, —OC(═O)R^{B3a}, —OC(═O)OR^{B3a}, or —OC(═O)N(R^{B3a})₂, wherein each instance of R^{B3a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{B3a} groups are joined to form a substituted or unsubstituted heterocyclic or substituted or unsubstituted heteroaryl ring;
p is 0 or an integer between 1 and 4, inclusive;
L¹ a bond,

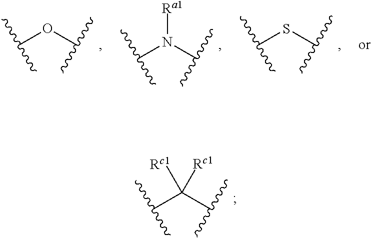

R^{a1} is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or a nitrogen protecting group; and
each instance of R^{c1} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{c1a}, —N(R^{c1a})₂, —SR^{c1a}, —CN, —C(═O)R^{c1a}, —C(═O)OR^{c1a}, —C(═O)N(R^{c1a})₂, —NR^{c1a}C(═O)R^{c1a}, —NR^{c1a}C(═O)OR^{c1a}, —NR^{c1a}C(═O)N(R^{c1a})₂, —OC(═O)R^{c1a}, or —OC(═O)N(R^{c1a})₂, wherein each instance of R^{c1a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or about two R^{c1a} groups are joined to form a substituted or unsubstituted heterocyclic or substituted or unsubstituted heteroaryl ring.

In certain embodiments, the bromodomain inhibitor is of the formula:

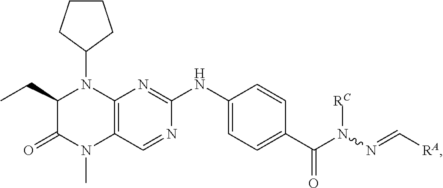

or a pharmaceutically acceptable salt, solvate, hydrate, polymorph, co-crystal, tautomer, stereoisomer, isotopically labeled derivative, or prodrug thereof, wherein R^{A} is selected from Table 1.

In certain embodiments, the bromodomain inhibitor is of the formula:

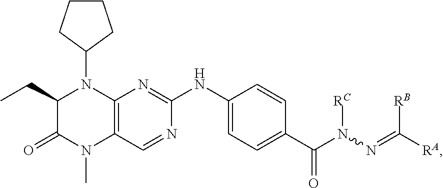

or a pharmaceutically acceptable salt, solvate, hydrate, polymorph, co-crystal, tautomer, stereoisomer, isotopically labeled derivative, or prodrug thereof, wherein R^{A} and R^{B} are independently selected from Table 2.

In certain embodiments, the bromodomain inhibitor is of the formula:

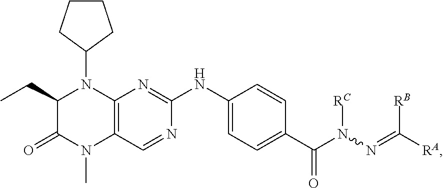

or a pharmaceutically acceptable salt, solvate, hydrate, polymorph, co-crystal, tautomer, stereoisomer, isotopically labeled derivative, or prodrug thereof, wherein

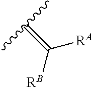

is selected from Table 3.

### Immune Modulators

Certain aspects of the invention relate to the surprising discovery that combinations of bromodomain inhibitors and immune modulators (e.g., immune checkpoint inhibitors) are particularly effective in treating cancers. In some embodiments, the immune modulator activates expression or activity of a stimulatory immune molecule. In some embodiments, the stimulatory immune molecule is selected from the group consisting of 4-1BB (CD137), CD137L, OX40, OX40L, ICOS, CD40, CD40L, CD70, CD27, CD28, CD80, CD86, B7RP1, and Herpesvirus entry mediator (HVEM). In some embodiments, the immune modulator is a peptide, antibody, interfering RNA, or small molecule. In some embodiments, the immune modulator is a monoclonal antibody, or an Ig fusion protein. In some embodiments, the immune modulator is an agonistic antibody directed to a stimulatory immune molecule (e.g., 4-1BB (CD137), CD137L, OX40, OX40L, ICOS, CD40, CD40L, CD70, CD27, CD28, CD80, CD86, B7RP1, or HVEM).

In some embodiments, the immune modulator inhibits expression or activity of an inhibitory immune molecule (e.g., an immune checkpoint molecule). In some embodiments, the immune modulator is an immune checkpoint inhibitor.

As used herein, the term “immune checkpoint inhibitor” refers to an agent that reduces, slows, halts, and/or prevents activity of a an immune checkpoint protein in a cell relative to vehicle. Immune checkpoint proteins are proteins that regulate the inhibitory pathways of a subject's (e.g. human's) immune system, maintain self-tolerance, and modulate the duration and amplitude of a physiological immune response. Typically, immune checkpoint proteins are dysregulated by cancer cells (e.g., tumors). Without wishing to be bound by any particular theory, immune checkpoint proteins can be targeted with inhibitors as an anti-cancer therapy, for example as described by Pardoll et al., Nature Reviews Cancer, 12: 252-264, 2012.

Non-limiting examples of immune checkpoint proteins include inhibitory receptors and their cognate ligands. Examples of inhibitory receptors include, but are not limited to, Cytotoxic T-cell-Lymphocyte-associated Antigen 4 (CTLA4), Programmed Cell Death protein 1 (PD1), Lymphocyte Activation Gene 3 (LAG3), T-cell Membrane Protein 3 (TIM3), and 4-1BB (CD137). Examples of immune checkpoint proteins that are ligands include, but are not limited to, PD1 Ligands 1 and 2 (PDL-1, PDL-2), B7-H3, B7-H4, and 4-1BB (CD137) ligand. Thus, in some embodiments, the immune checkpoint inhibitor is an inhibitor of an immune checkpoint protein selected from the group consisting of: CTLA-4, PD-1, PDL-1, TIM3, LAG3, B7-H3, B7-H4, and 4-1BB (CD137).

An immune checkpoint inhibitor can be a peptide, antibody, interfering RNA, or small molecule. Generally, immune checkpoints are initiated by ligand-receptor interactions between immune checkpoint proteins. See, for example, Pardoll et al., Nature Reviews Cancer, 12: 252-264, 2012. In some embodiments, such interactions are blocked by using specific antibodies (e.g., antibodies that bind specifically to an immune checkpoint protein or its interacting partner), recombinant protein ligands, and/or soluble recombinant receptor proteins. Thus, in some embodiments, the immune checkpoint inhibitor is an antibody (e.g., a monoclonal antibody), or an Ig fusion protein.

Methods of producing antibodies are well known in the art. For example, an epitope of a target protein (e.g., an immune checkpoint protein) can be used to generate polyclonal antibodies in animals. Alternatively, a monoclonal antibody can be produced. Methods of producing monoclonal and polyclonal antibodies are described, for example, in Antibodies: A Laboratory Manual, Harlow and Lane, Cold Spring Harbor Laboratory, New York, 1988. Examples of antibody immune checkpoint inhibitors include Ipilimumab, Tremelimumab, MDX-1106 (BMS-936558), MK3475, CT-011 (Pidilizumab), MDX-1105, MPDL3280A, MEDI4736, and MGA271. Further examples of antibody immune checkpoint inhibitors are disclosed, in Creelan, Cancer Control, 21(1):80-89, 2014. In some embodiments, the immune checkpoint inhibitor is selected from the group consisting of: anti-PD-1 antibody and anti-4-1BB antibody.

As used herein, the term “Ig fusion protein” refers to a recombinant protein that comprises the Fc domain of an immunoglobulin (Ig) linked to a peptide or protein of interest. Generally, the Fc domain of an Ig fusion protein increases bioavailability and in vivo half-life of the peptide or protein of interest. In some embodiments, an Ig fission protein comprises a peptide or protein that is a ligand (e.g., PDL-1) of an immune checkpoint protein (e.g. an immune checkpoint receptor, such as PD1) and is thus configured to inhibit said immune checkpoint protein. Examples of Ig fusion protein immune checkpoint inhibitors include AMP-224 and IMP321. Other suitable Ig fusion protein immune checkpoint inhibitors can be produced by methods known in the art, for example as disclosed in Cannon et al., Methods Mol. Biol., 748:51-67, 2011.

### Pharmaceutical Compositions and Modes of Administration

Pharmaceutical compositions described herein can he prepared by any method known in the art of pharmacology. In general, such preparatory methods include bringing the bromodomain inhibitors and/or immune modulators (e.g., immune checkpoint inhibitors) described herein (i.e., the “active ingredients”) into association with a carrier or excipient, and/or one or more other accessory ingredients, and then, if necessary and/or desirable, shaping, and/or packaging the product into a desired single- or multi-dose unit. Pharmaceutical compositions provided herein can be produced in a manner known to the skilled artisan as described, for example, in Remington's Pharmaceutical Sciences, 15th Ed., Mack Publishing Co., New Jersey (1991).

The bromodomain inhibitors and/or immune modulators provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The bromodomain inhibitors, immune modulators, and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site (e.g., a solid organ tumor). In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the bromodomain inhibitors, immune modulators, and pharmaceutical compositions described herein are suitable for topical administration to the eye of a subject.

The exact amount (e.g., combined amount) of a bromodomain inhibitor and an immune modulator required to achieve an effective amount will vary from subject to subject, depending, for example, on species, age, and general condition of a subject, severity or the side effects or disorder, identity of the particular bromodomain inhibitor, identity of the particular immune checkpoint inhibitor, mode of administration, and the like. An effective amount may be included in a single dose (e.g., single oral dose) or multiple doses (e.g., multiple oral doses). In some embodiments, each dose is a combination of the bromodomain inhibitor and the immune modulator. In some embodiments, the combination of the bromodomain inhibitor and the immune modulator is administered as a single composition (e.g., a heterogeneous mixture of the two inhibitors). In some embodiments, the bromodomain inhibitor and the immune modulator may be independently administered (e.g., individually administered as separate compositions) at the same time or administered separately at different times in any order. For example, a bromodomain inhibitor can be administered prior to, concurrently with, or after administration of an immune modulator.

In certain embodiments, the duration between an administration of the bromodomain inhibitor and an administration of the immune modulator is about one hour, about two hours, about six hours, about twelve hours, about one day, about two days, about four days, or about one week, wherein the administration of the bromodomain inhibitor and the administration of the immune modulator are consecutive administrations. In some embodiments, an administration of a bromodomain inhibitor is occurs at least 24 hours (1 day), 2 days, 3 days, or 4 days prior to the administration of an immune modulator.

In some aspects, the invention relates to administering, a therapeutically effective amount of a bromodomain inhibitor and an immune modulator to a subject. An “effective amount” refers to an amount sufficient to elicit the desired biological response, e.g., treating cancer. As will be appreciated by those of ordinary skill in this art, the effective amount of the compounds described herein may vary depending on such factors as the desired biological endpoint, the pharmacokinetics of the compound, the condition being treated, the mode of administration, and the age and health of the subject. An effective amount includes, but is not limited to, that amount necessary to slow, reduce, inhibit, ameliorate or reverse one or more symptoms associated with cancer. For example, in the treatment of cancer, such terms may refer to a reduction in the size of the tumor.

In some embodiments, an effective amount is an amount of agent (e.g., bromodomain inhibitor and/or an immune modulator) that results in a reduction of expression and/or activity of the protein to be inhibited (e.g., a bromodomain-containing protein and/or an immune checkpoint protein) in the cancer cells. The reduction in expression and/or activity resulting from administration of an effective amount of bromodomain inhibitor and/or immune checkpoint inhibitor can range from about 2-fold to about 500-fold, 5-fold to about 250-fold, 10-fold to about 150-fold, or about 20-fold to about 100-fold. In some embodiments, reduction in expression and/or activity of a bromodomain-containing protein and/or an immune checkpoint protein) resulting from administration of an effective amount of inhibitor (e.g., bromodomain inhibitor and/or an immune checkpoint inhibitor) can range from about 100% to about 1%, about 90% to about 10%, about 80% to about 20%, about 70% to about 30%, about 60% to about 40%. In some embodiments, an amount effective to treat the cancer results in a cell lacking expression and/or activity of a brotnod.omain-containing protein and/or an immune checkpoint protein (e.g., complete silencing or knockout of a gene encoding a bromodomain-containing protein and/or a gene encoding an immune checkpoint protein). Inhibition of a bromodomain-containing protein and/or an immune checkpoint protein can be measured by any suitable means known in the art. For example, protein level can be measured by Western blot or gene expression level can be measured by quantitative PCR (qPCR). In another example, inhibition of a bromodomain-containing protein can be measured by assaying functional activity (e.g., activity of proteins controlled or regulated by a bromodomain-containing protein) in a subject. In another example, inhibition of an immune checkpoint protein can be measured by assaying functional activity (e.g., changes in immune cell activation or stimulation) in a subject.

An effective amount of a compound (e.g., a bromodomain inhibitor or an immune checkpoint inhibitor) may vary from about 0.001 mg/kg to about 1000 mg/kg in one or more dose administrations, for one or several days (depending on the mode of administration). In certain embodiments, the effective amount varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg. One of ordinary skill in the art would be able to determine empirically an appropriate therapeutically effective amount.

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds provided herein may be administered at dosage levels sufficient to deliver from about 0.001 mg/kg to about 100 mg/kg, from about 0.01 mg/kg to about 50 mg/kg, preferably from about 0.1 mg/kg to about 40 mg kg, preferably from about 0.5 mg kg to about 30 mg/kg, from about 0.01 mg kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### EXAMPLES

### Materials and Methods

### Cell Lines and Reagents

Eμ-Myc lymphomas were derived, cultured and transplanted as previously described [1]. Retroviral transduction of freshly isolated Eμ-Myc lymphomas with murine stern-cell virus-internal ribosomal entry site-green fluorescence protein (MSCV-IRES-GFP) and Bcl2 (MSCV-IRES-GFP/Bcl-2) constructs were performed as previously described [2]. Retroviral TRMPVIR Tet-shRNA expression vectors were transfected into HEK293T Phoenix packaging cells using standard calcium phosphate transfection protocols. Viral supernatant was used to transduce Eμ-Myc lymphoma cells (^{#}4242) in RetroNectin (TaKaRa, Shiga, Japan)-pre-coated 6-well plates (Becton Dickinson, Franklin Lakes, N.J.). After 72 hours. GFP-positive cells were sorted by flow cytometry and expanded in vitro. GFP-positive cells were treated in vitro with 1 μg/mL doxycycline (Dox, Sigma-Aldrich) to induce shRNA/DsRed expression. All human cell lines were maintained at 5% CO₂ and cultured in Gibco RPMI-1640 supplemented with 10% fetal calf serum, penicillin (100 u/mL), and streptomycin (100 mg/mL). Recombinant human interferon-gamma (IFN-γ) was purchased from BD Pharmingen (San Diego, Calif.). For in vitro use, JQ1, IBET-151, IBET-762, RVX-208, Y803, dBET1 was dissolved in dimethylsulfoxide (DMSO) to a final concentration of 10 mM.

### In Vitro Drug Treatment

Eν-Myc lymphoma cells (5×10⁵) were incubated in the presence of JQ1, or DMSO, in 500 μL culture media in a 48 well plates (Corning, N.Y.) prior to analysis of PD-L1/L2 expression by flow cytometry. Human RPMI-8226 and L540 cells (5×10⁵) were incubated in the presence of JQ1, or DMSO vehicle, in 500 μL culture media in a 48 well plates (Corning, N.Y.). Additionally RPMI-8226 cells were cultured with 100 ng/ML, IFN-γ as a single agent and in combination with JQ1, prior to analysis of PD-L1/L2 expression by flow cytometry.

### Flow Cytometry

Cell suspensions were washed once with ice-cold flow cytometry buffer (2% FCS and 0.02% NaN₃ in PBS) and resuspended in anti-CD16/32 monoclonal antibody (clone 2.4G2) on ice for 30 minutes to block Fc receptors. Cell suspensions were washed once with ice-cold flow cytometry buffer and stained on ice for 30 minutes with the following conjugated antibodies: anti-mouse CD3 (pacific blue, 1:400, clone 17A2), anti-mouse CD4 (APC, 1:400, clone RM4-5), anti-mouse CD8 (PE-Cy7, 1:400, clone 53-6.7), anti-mouse PD-1 (FITC, 1:400, clone J43), anti-mouse PD-L1 (PE, 1:100, clone MIH5), anti-mouse PD-L2 (Biotin, 1:200, clone TY25), anti-human PD-L1 (PE, 1:200, clone 29E.2A3), or anti-human PD-L2 (Biotin, 1:200, clone 24F.10C12). Biotinylated antibodies were subsequently incubated with Streptavidin-PE-Cy7 (1:1000, catalogue number 25-4317-82) or Streptavidin-Pacific Blue (1:1000, catalogue number 48-4317-82) for 30 minutes on ice. Anti-Armenian Hamster IgG (FITC, 1:400, catalogue number 554011), Mouse IgG2aκ (PE, 1:100, clone RTK2758), Rat IgG2aκ (Biotin, 1:200, clone eBR2a), Mouse IgG2bκ (PE, 1:200, catalogue number 559529), and Mouse IgG2aκ (Biotin, 1:200, clone eBM2a) were used as isotype control antibodies, respectively. All antibodies were purchased from BioLegend (San Diego, Calif.), eBiosciences (San Diego, Calif.), or BD Bioscience (San Diego, Calif.). Cell suspensions were washed once with ice-cold flow cytometry buffer, resuspended in ice-cold flow cytometry buffer containing 7-AAD (1:1000, BD Bioscience) and analyzed by flow cytometry. Data was collected on a LSR Fortessa flow cytometer (BD Biosciences) and analyzed using FlowJo Software, version 10.0.7 (Tree Star).

### Quantitative Real-Time PCR

Eμ-Myc lymphomas cells were cultured in the presence of JQ1, or DMSO, as described above. RNA was extracted from cell pellets using the Nucleospine® RNA extraction kit (Macherey-Nagel, Bethlehem, Pa.) as per the manufacturer's instructions. cDNA was synthesized according to the manufacturer's instructions (Promega, Sydney, NSW). Quantitative PCR analysis of samples was performed on the 7900HT Fast Real-Time PCR System (Applied Biosystems, Mulgrave, VIC, Australia') with SYBR-green ROX mix (Agilent, Mulgrave, VIC, Australia). GAPDH was used as the murine control genes. Primer sequences were: Mus musculus PD-L1 F: TTCGTACGGGCGTTTACTATC (SEQ ID NO: 1) R: TCCCGTTCTACAGGGAATCT (SEQ ID NO: 2), Mus musculus GAPDH F: CCTTCATTGACCTCAACTAC (SEQ ID NO: 3) R: GGAAGGCCATGCCAGTGAGC (SEQ ID NO: 4).

### Chromatin Immunoprecipitation-PCR

ChIP studies were carried out using 5×10⁷ tumor cells treated 2 hours with 1 μM JQ1 or DMSO control and crosslinked for 10 minutes at room temperature by the addition of one-tenth of the volume of 11% formaldehyde solution (11% formaldehyde, 50 mM HEPES pH 7.3, 100 mM NaCl, 1 mM EDTA pH 8.0, 0.5 mM, EGTA pH8.0) followed by quenching with 0.125M glycine and two washes with PBS. Fifty μl of Dynal protein 6 magnetic beads (Sigma) were blocked with 0.5% BSA (w/v) in PBS. Magnetic beads were bound with 10 μg of the anti-BRD4 antibody (Bethyl Labs #A301-985A). Crosslinked cells were lysed with lysis buffer 1 (50 mM HEPES-KOH pH 7.5, 140 mM NaCl, 1 mM EDTA pH 8.0, 10% glycerol, 0.5% NP-40, and 0.25% Triton X-100) and washed with lysis buffer 2 (10 mM Tris-HCl pH 8.0, 200 mM NaCl, 1 mM EDTA pH 8.0, and 0.5 mM EGTA pH 8.0). Cells were resuspended and sonicated in lysis buffer 3 (50 mM HEPES-KOH pH 7.5, 140 mM NaCl, 1 mM EDTA pH 8.0, 1 mM EGTA pH 8.0, 1% Triton X-100, 0.1% NaDeoxycholate and 1% SDS) for 4× 10 minute cycles, 30 second on/off cycles using a Bioruptor sonicator on power setting HIGH, Sonicated lysates were cleared, diluted 1:10 with dilution buffer (50 mM HEPES-KOH pH 7.5, 140 mM NaCl, 1 mM EDTA pH 8.0, 1 mM EGTA pH 8.0, 1% Triton X-100, 0.1% Na-Deoxycholate) and incubated overnight at 4° C., with magnetic beads bound with antibody. Beads were washed two times with lysis buffer 3, once with high salt wash (50 mM HEPES-KOH pH 7.5, 500 mM NaCl, 1 mM EDTA pH 8.0, 1 mM EGTA pH 8.0, 1% Triton X-100, 0.1% Na-Deoxycholate and 0.1% SDS), once with LiCl wash buffer (20 mM Tris-HCl pH 8.0, 1 mM EDTA pH 8.0, 250 mM LiCl, 0.5% NP-40, 0.5% Na-deoxycholate), and once with TE buffer (10 mM Tris-HCl pH 8.0, 1 mM EDTA pH 8.0). Protease inhibitors (Roche Complete) were added to all lysis and wash buffers. Bound complexes were eluted twice in elution buffer (50 mM Tris-HCl pH 8.0, 10 mM EDTA pH 8.0, 1% SDS) at 65° C. for 15 min with occasional vortexin.g. Crosslinks were reversed. overnight at 65° C. RNA and protein were digested using RNase A and Proteinase K, respectively, and DNA was purified with phenol chloroform extraction and ethanol precipitation. Primers were designed to amplify regions of the murine PD-L1 locus: Promoter site 1 (forward) 5′-TCGACAGCCTCTCAGTAGCA-3′ (SEQ ID NO: 5) and (reverse) 5′-TGACACACGCCTTAATTCCA-3′(SEQ ID NO: 6); Enhancer site 1 (forward) 5′-ACCGGTTTCATGGAAGAATG-3′ (SEQ ID NO: 7) and (reverse) 5′-TTCACTCGGCAAACACTGAG-3′ (SEQ ID NO: 8); Enhancer site 2 (forward) 5′-GGTCCTTGGCTGAGITTGAA-3′ (SEQ ID NO: 9) and (reverse) 5′-GCCATGTAGAACCAAGTGGAA-3′(SEQ ID NO: 10) ; Enhancer site 3 (forward) 5′-CTCGGTTCTCCCTTTCACAG-3′ (SEQ ID NO: 11) and (reverse) 5′-CCAGCAGGACGTTCTTTCTC-3′(SEQ ID NO: 12): Enhancer site 4 (forward) 5′-CGCAGAGTGGATTTGAAACA-3′ (SEQ ID NO: 13) and (reverse) 5′-CAGCCAGGGAGAAAAGTGAC-3′(SEQ ID NO: 14); Enhancer site 5 (forward) 5′-TGCTTGGTCTTCATCGTCAG-3′ (SEQ ID NO: 15) and (reverse) 5′-ATACCCCACCTGGCCTACTC-3′ (SEQ ID NO: 16); Enhancer site 6 (forward) 5′-TGACAATGGTACAGAGAGATCACA-3′ (SEQ ID NO: 17) and (reverse) 5′-GCTCTGGGTTCTTGCTGATG-3′ (SEQ ID NO: 18); Enhancer site 7 (forward) 5′-GGGAGCAAAATGCAGTAAGAA-3′ (SEQ ID NO: 19) and (reverse) 5′-ATCGATGTGCGTAGCTTTCA-3′(SEQ ID NO: 20); Negative control region 1 (forward) 5′-CACTGCAACTGCCAGAGAAA-3′ (SEQ ID NO: 21) and (reverse) 5′-TCCAGACTCTTGGGGTATTCA-3′ (SEQ ID NO: 22); Negative control region 2 (forward) 5-CCCGTCTATGAAAGCAGGAG-3′ (SEQ ID NO: 23) and (reverse) 5′-CACGGGGATTGTTTAAATGC-3′ (SEQ ID NO: 24). Enrichment data were analyzed by calculating the immunoprecipitated DNA percentage of input DNA for each sample and normalizing to negative control region enrichment.

### In Vivo Analysis

Female C57BL/6 mice were purchased. C57BL/6.Rag2cγ^{-/-} mice were bred in house. C57BL/6.Rag1^{-/-} mice were purchased. For transplantation of Eμ-Myc lymphomas in vivo, cohorts of six- to eight-week-old syngeneic mice were inoculated via tail vein injection with 1-4×10⁵ Eμ-Myc lymphoma cells. Mice were treated with 50 mg/kg JQ1, reconstituted in 1 part DMSO to 9 parts 10% (w/v) Hydroxypropyl-β-cyclodextrin (HPBCD; Cyclodextrin Technologies Development Inc., Gainesville, Fla.) In sterile water, or DMSO vehicle control. Mice were dosed once daily (5 days/week) via intra-peritoneal (i.p.) injection, commencing three days post-intravenous inoculation, for a total of 5 weeks therapy or until treatment failure. Tumor-bearing C57BL/6 mice were treated with the following monoclonal antibodies via i.p. injection: Anti-4-1-BB (Anti-CD137, 100 μg, 3H3; BioXCell), anti-PD-1 (100 μg, RPMI-14; BioXCell), or control immunoglobulin. (cIg, Rat IgG2a, 2A3, 100 μg; BioXCell). Anti-PD-1 mAb was dosed on days 5, 10, 15, and 20 post tumor transplant. Anti-4-1BB mAb was dosed on days 5, 8, and 11 post tumor transplant.

### Statistical Analysis

Statistical analysis was performed using GraphPad Prism software, Version 6.0c (La Jolla, Calif.).

### An Intact Host Immune System is Required for the Robust Anti-Cancer Effects of JQ1 Against a Murine Model of Aggressive B-Cell Lymphoma

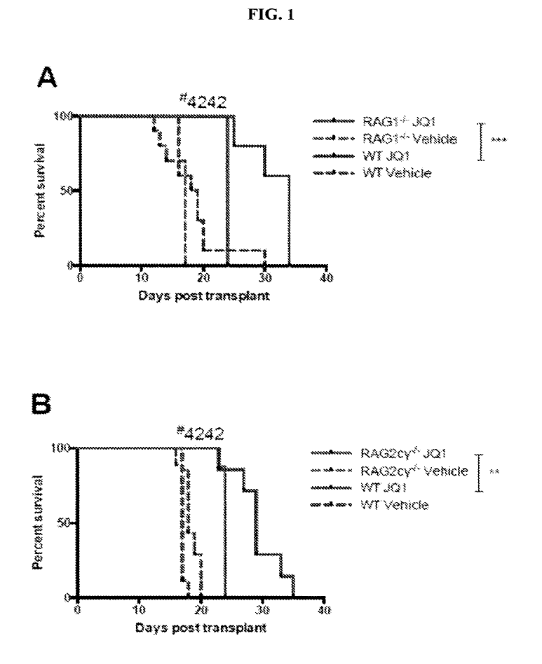

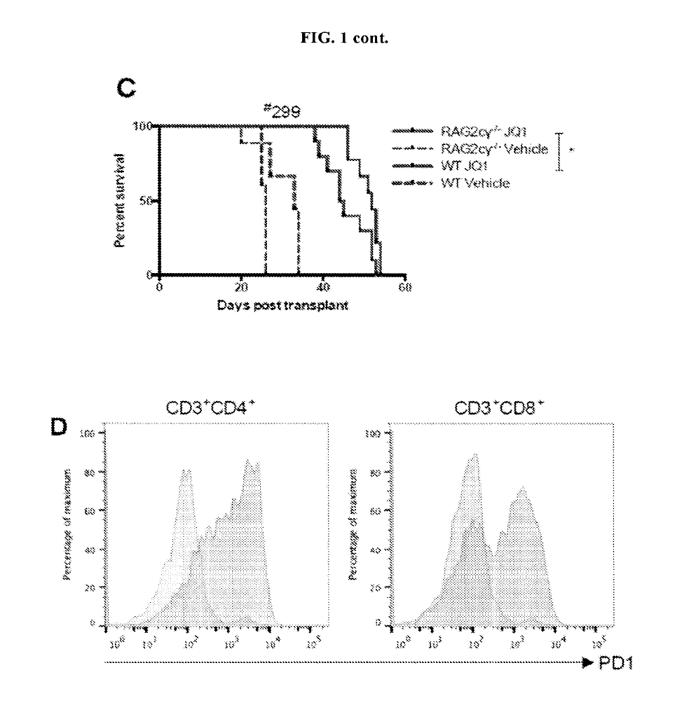

Cohorts of mice on a C57BL/6 background (n=10 per treatment group) were injected intravenously with 1-5×10⁵ Eμ-Myc lymphoma cells three days prior to commencement of daily dosing with JQ1 (50 mg/kg), or DMSO vehicle., via i.p. injection. FIGS. 1A and 1B show Kaplan-Meier survival curves representing cohorts of wild type C57BL/6 mice and immune compromised strains. FIG. 1A shows C57BL/6.Rag2cγ^{-/-} mice and FIG. 1B shows C57BL/6.Rag1^{-/-}. Both sets of mice were inoculated with Eμ-Myc lymphoma ^{#}4242 and treated with JQ1, or DMSO vehicle. FIG. 1C shows Kaplan-Meier survival curves representing cohorts of wild type C57BL/6 mice and immune compromised strain C57BL/6.Rag2cγ^{-/-} inoculated with Eμ-Myc lymphoma ^{#}299 and treated with JQ1 (solid line), or DMSO vehicle (dashed line).

In all therapy experiments, JQ1 conveyed a significant survival advantage to both immune competent and immune deficient mice bearing established Eμ-Myc lymphoma. However, immune deficient mice (C57BL/6.Rag2cγ^{-/-} and C57BL/6.Rag1^{-/-}) succumbed to disease significantly earlier than tumor-bearing wild type mice despite JQ1 treatment.

Splenic T-cells from tumor bearing mice express high levels of PD1, indicative of an exhausted phenotype. The spleen was harvested from a wild type C57BL/6 mouse bearing established Eμ-Myc lymphoma (^{#}299) at end-stage, and splenic CD3⁺CD4⁺ and CD3⁺CD8⁺ cells were analyzed for the expression of PD-1 by flow cytometry. Results are shown in FIG. 1D.

### PD-L1 is a Direct Target of BET Inhibition In Vitro and In Vivo.

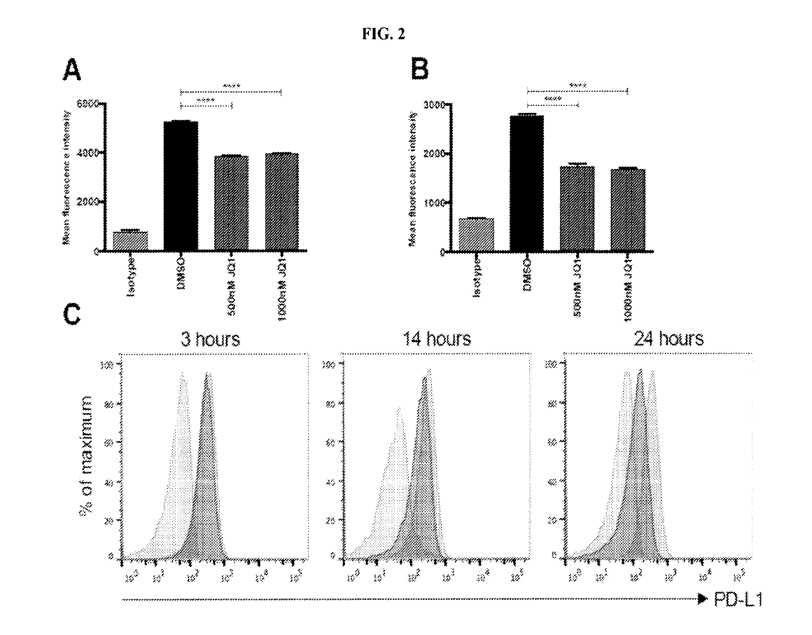

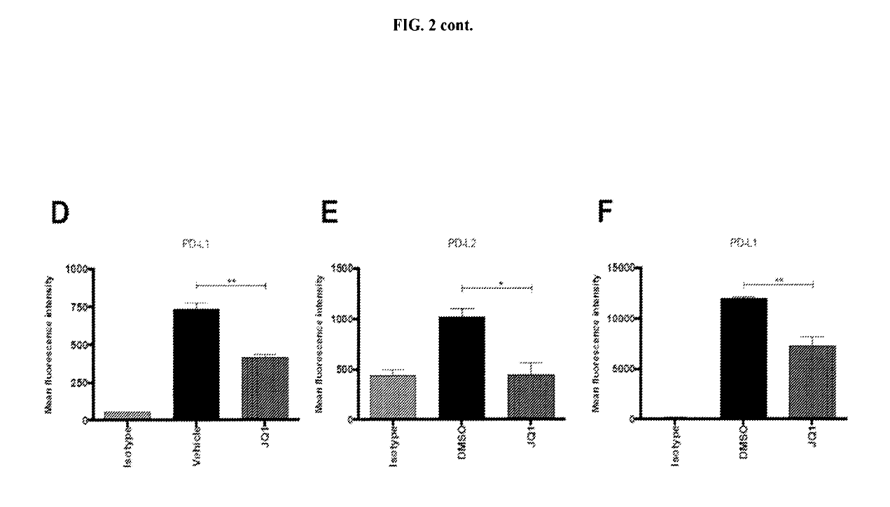

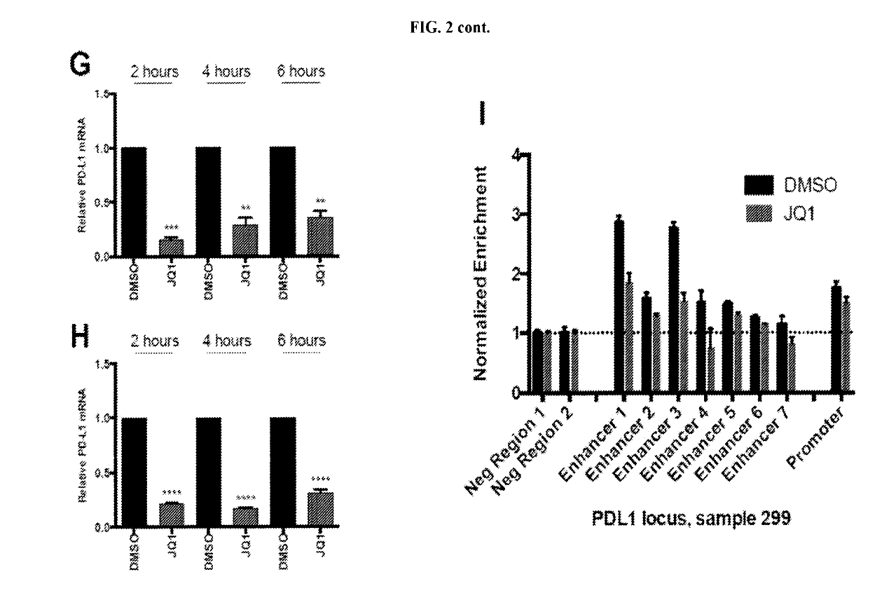

As described in FIGS. 2A and 2B, JQ1 downregulates the expression of PD-L1 (CD274) on lymphoma cells as determined by flow cytometry analysis. Graphs showing the mean fluorescence intensity (MFI) of PD-L1 on Eμ-Myc lymphoma cell lines (FIG. 2A) ^{#}4242 and (FIG. 2B) ^{#}299 over-expressing Bcl-2 following 24 hours treatment in vitro with indicated concentrations of JQ1, or DMSO control are provided. Representative data are presented as mean MFI of cells cultured and analyzed in triplicate±S.E.M. (****p <0.0001, Student's t test).

PD-L1 downregulation following BET inhibition is time-dependent. Flow cytometry analysis of PD-L1 expression on Eμ-Myc lymphoma cell line ^{#}6066 following treatment in vitro with 500 nM JQ1 or DMSO control for indicated time points was performed. Representative data are shown in FIG. 2C.

An acute dose of JQ1 in C57BL/6 mice bearing established Eμ-Myc lymphoma rapidly downregulates both PD-L1 and PD-L2 (CD273) on tumor cells. A cohort of mice were injected intravenously with 1-5×10⁵ Eμ-Myc lymphoma ^{#}4242 cells and left for 12 days to develop bulky nodal disease. Peripheral lymph nodes were harvested 16 hours following a single dose of JQ1 (50 mg/kg), or DMSO vehicle (n=3 per treatment group) and assessed by flow cytometry. Graphs show the MFI of (FIG. 2D) PD-L1 and (FIG. 2E) PD-L2 expression gated on live GFP-positive tumor cells. Data are presented as mean MFI from 3 individual mice=S.E.M. (*p<0.05, **p<0.01, Student's t test).

Circulating tumor cells from the peripheral blood of C57BL/6 mice bearing Eμ-Myc lymphoma and treated chronically with JQ1 express lower levels of PD-L1. A cohort of mice were injected intravenously with 1-5×10⁵ Eμ-Myc lymphoma ^{#}4242/Bcl-2 cells and treated daily with JQ1 (50 mg/kg), or DMSO vehicle (n=5 per treatment group). At day 18, peripheral blood was obtained and tumor cells were assessed by flow cytometry. FIG. 2F shows the MFI of PD-L1 expression gated on live GFP-positive tumor cells. Data are presented as mean MFI from 5 individual mice±S.E.M. (**p<0.01, Student's t test).

JQ1 rapidly downregulates PD-L1 transcript in vitro. Quantitative real-time-PCR (qPCR) analysis of PD-L1 mRNA levels in Eμ-Myc lymphoma cell lines (FIG. 2G) ^{#}4242 and (FIG. 2H) ^{#}299 over-expressing Bcl-2 following treatment with 1000 nM JQ1, or DMSO control, was performed at indicated time points. Transcript levels are presented as fold change compared to DMSO. Data are presented as mean fold-change from 3 separate experiments±S.E.M. (**p<0.01, ***p<0.001, ****p<0.0001, Student's t test).

FIG. 2I provides data for chromatin immunoprecipitation-PCR of Eμ-Myc lymphoma ^{#}299, and shows binding of BRD4 at the PD-L1 locus following 2 hours treatment in vitro with 1000 nM JQ1, or DMSO control.

### Genetic Knockdown of BRD4 Phenocopies BET Inhibitor Treatment.

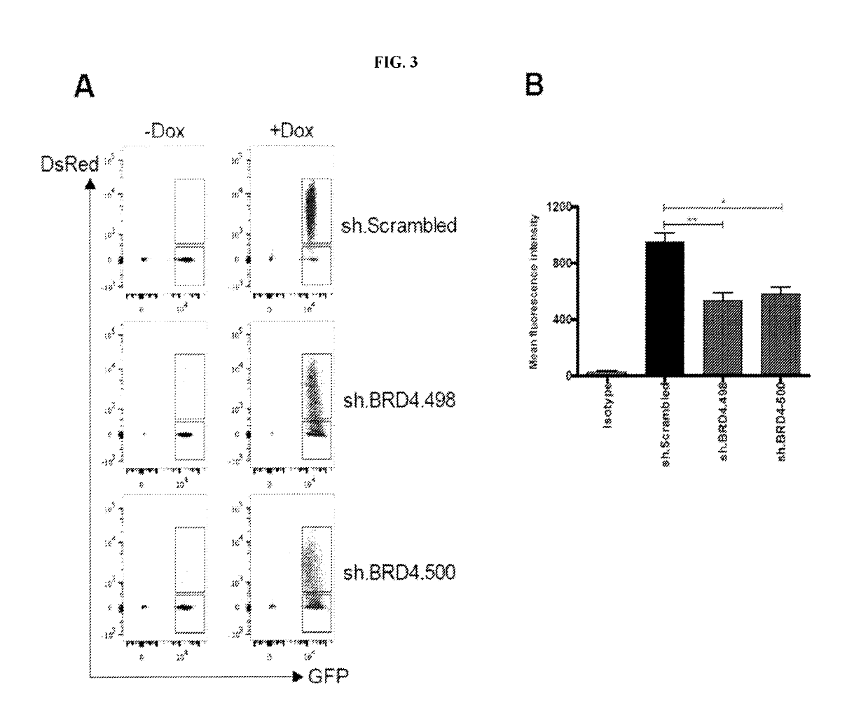

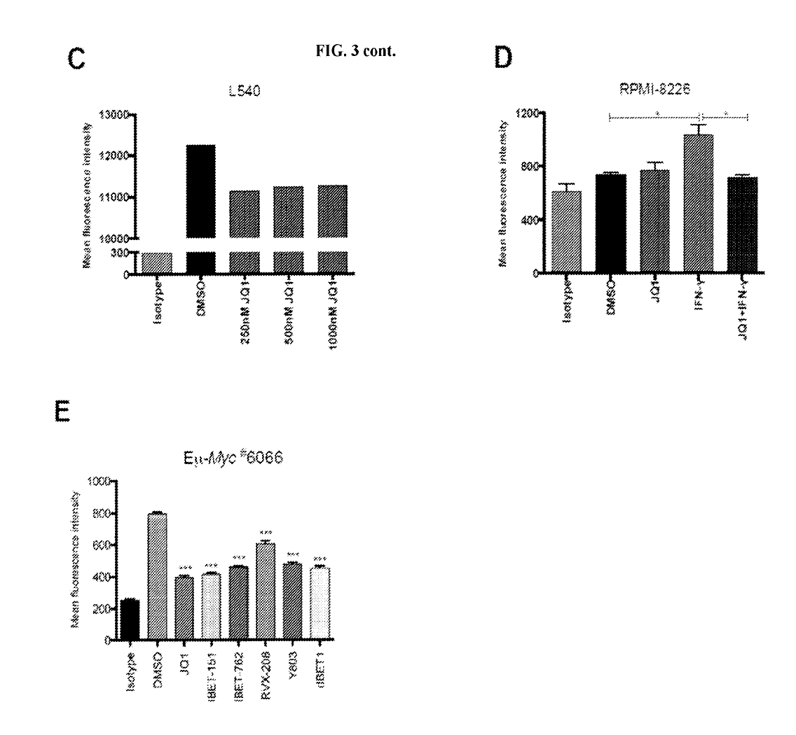

Eμ-Myc lymphoma cells (^{#}4242) were transduced with doxycycline (Dox)-inducible TRMPVIR shRNA expression vectors targeting BRD4 (sh.BRD4.498 and sh.BRD4.500) or scrambled control (sh.SCR). The PGK promoter drives constitutive GFP expression in retrovirally infected Eμ-Myc lymphoma cells and addition of Dox induces rtTA3 activity to activate the TRE promoter and the DsRed-shRNA gene cassette. FIG. 3A shows representative FACS plots of ^{#}4242 expressing sh.BRD4.498, sh.BRD4.500, and sh.SCR treated in the presence of absence of Dox for 16 hours in vitro.

Retrovirally infected and Dox-treated Eμ-Myc lymphoma cells expressing the indicated shRNA were gated on by GFP⁺DsRed⁺ cells. FIG. 3B shows the MFI of PD-L1 expression on GFP⁺DsRed⁺ populations following 16 hours in vitro treatment with Dox. Representative data are presented as mean MFI of cells cultured and analyzed in triplicate±S.E.M (*p<0.05, **p<0.01, Student's t test).

JQ1 treatment downregulates the constitutive expression of PD-L1 on human lymphoma cell lines. The Hodgkin lymphoma cell line L540, with a selective 9p24.1 amplification containing the PD-L1 loci, was treated for 24 hours in vitro with indicated concentrations of JQ1 prior to analysis with flow cytometry. FIG. 3C shows MFI of PD-L1 expression.

JQ1 treatment downregulates the IFN-γ-inducible expression of PD-L1 on human myeloma cell lines. The human Ig-cMYC translocated multiple myeloma cell line RPMI-8226 was treated with either single agent or combination of 100 ng/mL IFN-γ and 500 nM JQ1, or DMSO vehicle, for 24 hours prior to analysis by flow cytometry. FIG. 3D shows MFI of PD-L1 expression and IFN-γ-mediated induction of PD-L1 that can be abrogated with the co-treatment of JQ1. Representative data are presented as mean MFI of cells cultured and analyzed in triplicate=S.E.M. (*p<0.05, Student's t test).

Chemically distinct bromodomain inhibitors downregulate the expression of PD-L1 (CD274) on lymphoma cells, as shown by flow cytometry. FIG. 3E shows the mean fluorescence intensity (MFI) of PD-L1 on Eμ-Myc lymphoma cell line ^{#}6066 following 24 hours treatment in vitro with 1 μM JQ1, IBET-1.51, IBET-762, Y803, or dBET1, 10 μM RVX-208, or DMSO control. Representative data are presented as mean MFI of cells cultured and analyzed in triplicate±S.E.M. (***p<0.001, Student's t test).

JQ1 in Combination with Checkpoint Inhibitors or Immune Stimulating Antibodies Promotes Curative Anti-Tumor Responses.

FIGS. 4A and 4B show Kaplan-Meier survival curves representing cohorts of C56BL/6 (n=6 per treatment group) injected intravenously with 1-5×10⁵ Eμ-Myc lymphoma ^{#}299 cells. FIG. 4A shows the efficacy of JQ1 in combination with PD-1 blockade against Eμ-Myc lymphoma ^{#}299. Mice received JQ1 (50 mg/kg), or DMSO vehicle, via i.p. injection commencing day 3 post-transplant for a total of 25 doses. Mice received 100 μg of anti-PD-1 (clone RPMI-14) or Rat IgG isotype antibody via i.p. injection on days 5, 10, 15, and 20 post-transplant.

FIG. 4B shows the efficacy of JQ1 in combination with the agonistic anti-4-1BB (CD137) immune stimulating antibody against Eμ-Myc lymphoma ^{#}299. Mice received JQ1 (50 mg/kg), or DMSO vehicle, via i.p. injection from day 3 post-transplant for a total of 25 doses. Mice received 100 μg of anti-4-1BB (clone 3H3) or Rat IgG isotype antibody via i.p. injection on days 5, 8, and 11 post-transplant.

### REFERENCES

1. Whitecross, K. F., et al., Defining the target specificity of ABT-737 and synergistic antitumor activities in combination with histone deacetylase inhibitors. Blood, 2009. 113(9): p. 1982-1991.
2. Shortt, J., et al., Combined inhibition of PI3K-related DNA damage response kinases and mTORC1 induces apoptosis in MYC-driven B-cell lymphomas, Blood, 2013. 121(15): p. 2964-2974.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A method of treating cancer in a subject in need thereof, the method comprising administering to the subject a therapeutically effective amount of:
a bromodomain inhibitor; and,
an immune modulator,
wherein the bromodomain inhibitor is a compound represented by the following structural formula 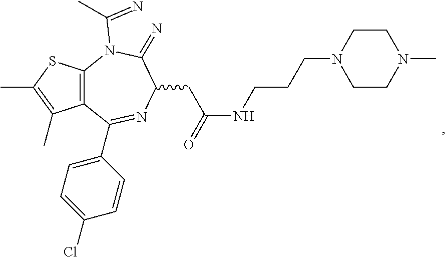
or a pharmaceutically acceptable salt thereof.

2-5. (canceled)

6. The method of claim 1, wherein the cancer is a hematological cancer or a solid organ tumor.

7. The method of claim 6, wherein the hematological cancer is lymphoma, leukemia, or myeloma.

8. The method of claim 6, wherein the solid organ tumor is a liver, colon, breast, kidney, head and neck, melanoma, skin, pancreas, lung, prostate, or brain tumor.

9-37. (canceled)

38. The method of claim 1, wherein the immune modulator is an immune checkpoint inhibitor.

39. The method of claim 1, wherein the immune checkpoint inhibitor is an inhibitor of an immune checkpoint protein selected from the group consisting of: CTLA-4, PD-1, PDL-1, TIM3, LAG3, B7-H3, B7-H4, BTLA, GAL9, and A2aR.

40. (canceled)

41. (canceled)

42. The method of claim 39, wherein the immune checkpoint inhibitor is an inhibitor of PDL-1.

43-53. (canceled)

54. The method of claim 1, wherein the bromodomain inhibitor and the immune modulator are administered to the subject simultaneously as a single composition.

55. The method of claim 1, wherein the bromodomain inhibitor and the immune modulator are administered to the subject separately.

56. The method of claim 1, wherein the bromodomain inhibitor and the immune modulator are administered to the subject concurrently.

57. The method of claim 56, wherein the bromodomain inhibitor is administered to the subject after the immune modulator.

58. The method of claim 56, wherein the bromodomain inhibitor is administered to the subject prior to the immune modulator.

59. The method of claim 58, wherein the administration of the bromodomain inhibitor occurs at least 24 hours (1 day), 2 days, 3 days or 4 days prior to the administration of the immune modulator.

60. (canceled)

61. The method of claim 1, wherein the subject has an intact immune system.

62. The method of claim 1, wherein the subject is a human.

63. The method of claim 1, wherein the immune modulator is the anti-PD-L1 antibody is MPDL3280A.

64. The method of claim 1, wherein the bromodomain inhibitor is a compound represented by the following structural formula: 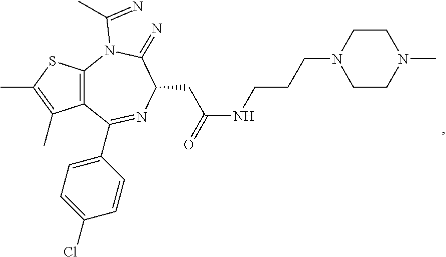
or a pharmaceutically acceptable salt thereof.

65. The method of claim 6, wherein the cancer is a solid organ tumor selected from an ovarian cancer.

66. The method of claim 6, wherein the cancer is a hematological cancer selected from acute lymphocytic leukemia (ALL), acute myelocytic leukemia (AML), chronic myelocytic leukemia (CML), Hodgkin lymphoma (HL), non-Hodgkin lymphoma (NHL), mantle cell lymphoma (MCL), B-cell lymphoma, and multiple myeloma.

67. The method of claim 1, wherein the immune modulator is an anti-PD-1 antibody or an anti-4-1BB antibody.
